(19) United States
(12) Patent Application Publication
(10) Pub. No.: US 2025/0255093 A1
(43) Pub. Date: Aug. 7, 2025

(54) COMPOUND AND ORGANIC LIGHT-EMITTING ELEMENT COMPRISING SAME

(71) Applicant: LG CHEM, LTD., Seoul (KR)

(72) Inventors: Jae Tak Lee, Daejeon (KR); Jung Min Yoon, Daejeon (KR); Heekyung Yun, Daejeon (KR); Su Jin Han, Daejeon (KR); Dong Uk Heo, Daejeon (KR); Sung Kil Hong, Daejeon (KR)

(21) Appl. No.: 18/855,759

(22) PCT Filed: Nov. 9, 2023
(86) PCT No.: PCT/KR2023/017986

§ 371 Date: Oct. 10, 2024

(30) Foreign Application Priority Data
Nov. 9, 2022 (KR) 10-2022-0148913

(51) Int. Cl. H10K 50/16; C07D 251/24; C07D 471/00; H10K 50/17; H10K 85/60

(52) U.S. Cl. CPC H10K 50/16; C07D 251/24; C07D 471/00; H10K 50/171; H10K 85/626; H10K 85/633; H10K 85/654; H10K 85/6572; H10K 85/658

(57) ABSTRACT

A compound of Chemical Formula 1:

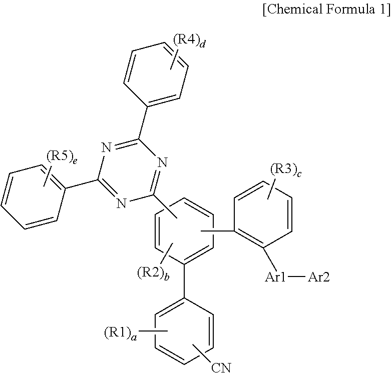

wherein: R1 to R5 are the same as or different from each other, and are each independently hydrogen, deuterium, a nitrile group, a halogen group, a substituted or unsubstituted alkyl group, a substituted or unsubstituted aryl group, or a substituted or unsubstituted heteroaryl group; Ar1 is a substituted or unsubstituted heteroaryl group including two or more N atoms; Ar2 is hydrogen, deuterium, a nitrile group, a halogen group, a substituted or unsubstituted alkyl group, a substituted or unsubstituted aryl group, a substituted or unsubstituted heteroaryl group, or a substituted or unsubstituted fused ring group; and the other substituents are as defined in the specification; and an organic light emitting device including the same. The device including the compound as a material in an organic material layer exhibits excellent characteristics in terms of efficiency, driving voltage, and/or stability.

### CROSS-REFERENCE TO RELATED APPLICATIONS

This application is a National Stage Application of International Application No. PCT/KR2023/017986 filed on Nov. 9, 2023, which claims priority to and the benefit of Korean Patent Application No. 10-2022-0148913 filed in the Korean Intellectual Property Office on Nov. 9, 2022, the entire contents of which are incorporated herein by reference.

### TECHNICAL FIELD

The present specification relates to a compound and an organic light emitting device including the same.

### BACKGROUND ART

In the present specification, an organic light emitting device is a light emitting device using an organic semiconductor material, and requires an exchange of holes and/or electrons between electrodes and organic semiconductor materials. The organic light emitting device may be roughly divided into the following two organic light emitting devices depending on the operation principle. The first organic light emitting device is a light emitting device in which an exciton is formed in an organic material layer by a photon that flows from an external light source to the device, the exciton is separated into electrons and holes, and the electrons and the holes are each transferred to different electrodes and used as a current source (voltage source). The second organic light emitting device is a light emitting device in which holes and/or electrons are injected into organic semiconductor material layers forming an interface with an electrode by applying a voltage or current to two or more electrodes, and the device is operated by the injected electrons and holes.

An organic light emission phenomenon generally refers to a phenomenon converting electrical energy to light energy using an organic material. An organic light emitting device using an organic light emission phenomenon normally has a structure including a positive electrode, a negative electrode, and an organic material layer therebetween. Here, the organic material layer has in many cases a multi-layered structure composed of different materials in order to improve the efficiency and stability of the organic light emitting device, and for example, may be composed of a hole injection layer, a hole transport layer, a light emitting layer, an electron blocking layer, an electron transport layer, an electron injection layer, and the like. In such a structure of the organic light emitting device, if a voltage is applied between the two electrodes, holes are injected from the positive electrode into the organic material layer and electrons are injected from the negative electrode into the organic material layer, and when the injected holes and electrons meet each other, an exciton is formed, and light is emitted when the exciton falls down again to a ground state. Such an organic light emitting device has been known to have characteristics such as self-emission, high luminance, high efficiency, a low driving voltage, a wide viewing angle, and high contrast.

In an organic light emitting device, materials used as an organic material layer may be classified into a light emitting material and a charge transport material, for example, a hole injection material, a hole transport material, an electron blocking material, an electron transport material, an electron injection material, and the like depending on the function. The light emitting materials include blue, green, and red light emitting materials according to the light emitting color, and yellow and orange light emitting materials required for implementing a much better natural color.

Furthermore, a host/dopant system may be used as a light emitting material for the purpose of enhancing color purity and light emitting efficiency through energy transfer. The principle is that when a small amount of dopant which has a smaller energy band gap and better light emitting efficiency than those of a host mainly constituting a light emitting layer is mixed in the light emitting layer, the excitons generated by the host are transported to the dopant to emit light with high efficiency. In this case, it is possible to obtain light with a desired wavelength according to the type of dopant used because the wavelength of the host moves to the wavelength range of the dopant.

In order to fully exhibit the above-described excellent characteristics of the organic light emitting device, a material constituting an organic material layer in a device, for example, a hole injection material, a hole transport material, a light emitting material, an electron blocking material, an electron transport material, an electron injection material, and the like need to be supported by stable and efficient materials, so that there is a continuous need for developing a new material.

### BRIEF DESCRIPTION

### Technical Problem

The present specification describes a compound and an organic light emitting device including the same.

### Technical Solution

An exemplary embodiment of the present specification provides a compound of the following Chemical Formula 1.

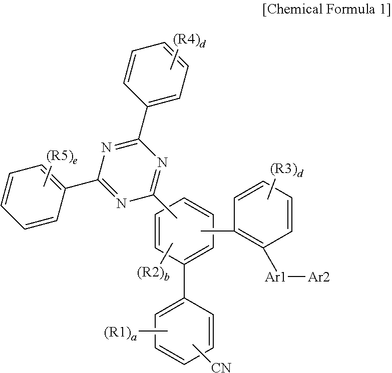

In Chemical Formula 1,
R1 to R5 are the same as or different from each other, and are each independently hydrogen, deuterium, a nitrile group, a halogen group, a substituted or unsubstituted alkyl group, a substituted or unsubstituted aryl group, or a substituted or unsubstituted heteroaryl group,
Ar1 is a substituted or unsubstituted heteroarylene group including two or more N atoms,
Ar2 is hydrogen, deuterium, a nitrile group, a halogen group, a substituted or unsubstituted alkyl group, a substituted or unsubstituted aryl group, a substituted or unsubstituted heteroaryl group, or a substituted or unsubstituted fused ring group,
a and c are an integer from 1 to 4,
b is an integer from 1 to 3,
d and e are an integer from 1 to 5,
when a is 2 or greater, the R1s are the same as or different from each other,
when b is 2 or greater, the R2s are the same as or different from each other,
when c is 2 or greater, the R3s are the same as or different from each other,
when d is 2 or greater, the R4s are the same as or different from each other, and
when e is 2 or greater, the R5s are the same as or different from each other.

Further, an exemplary embodiment of the present specification provides an organic light emitting device including: a first electrode; a second electrode provided to face the first electrode; and an organic material layer having one or more layers provided between the first electrode and the second electrode, in which one or more layers of the organic material layer include the above-described compound.

### Advantageous Effects

The compound of the present invention can be used as a material for an organic material layer of an organic light emitting device. When an organic light emitting device is manufactured by including the compound of the present invention, an organic light emitting device having high efficiency, low voltage and long-service life characteristics can be obtained, and when the compound of the present invention is included in an electron transport layer of an organic light emitting device, an effect of transferring electrons is high due to high intramolecular polarization, so that an organic light emitting device having long-service life characteristics can be manufactured.

Instead of narrowing the distance between electron transport groups, the compound of the present invention maximizes electron mobility by limiting the substitution position of a linking group to the ortho direction to cut the intramolecular conjugation appropriately, thereby exhibiting high efficiency characteristics, and by attaching a CN group, which is a substitution group having a high electronegativity, the compound of the present invention also allows long-service life characteristics to be maintained.

### BRIEF DESCRIPTION OF THE DRAWINGS

FIGS. 1 to 2 illustrate an example of the organic light emitting device according to the present invention.

### EXPLANATION OF REFERENCE NUMERALS AND SYMBOLS

1: Substrate
2: Positive electrode
3: Organic material layer
4: Negative electrode
5: Hole injection layer
6: Hole Transfer Layer
7: Electron blocking layer
8: Light Emitting Layer
9: Hole blocking layer
10: Electron injection and transport layer

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

When one member is disposed “on” another member in the present specification, this includes not only a case where the one member is brought into contact with another member, but also a case where still another member is present between the two members.

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, the term “substituted or unsubstituted” means being substituted with one or two or more substituents selected from the group consisting of deuterium; a halogen group; a cyano group (—CN); a silyl group; a boron group; a substituted or unsubstituted alkyl group; a substituted or unsubstituted cycloalkyl group; a substituted or unsubstituted aryl group; and a substituted or unsubstituted heterocyclic group or being substituted with a substituent to which two or more substituents are linked together among the substituents exemplified above, or having no substituent. For example, “the substituent to which two or more substituents are linked together” may be a biphenyl group. That is, the biphenyl group may also be an aryl group, and may be interpreted as a substituent to which two phenyl groups are linked together.

Examples of the substituents will be described below, however, the substituents are not limited thereto.

In the present specification, examples of a halogen group include fluorine (F), chlorine (Cl), bromine (Br) or iodine (I).

In the present specification, a silyl group may be unsubstituted or substituted with deuterium; a substituted or unsubstituted alkyl group; a substituted or unsubstituted aryl group, or the like. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group may be unsubstituted or substituted with deuterium; a substituted or unsubstituted alkyl group; or a substituted or unsubstituted aryl group. Specific examples of the boron group include a trimethylboron group, a triethylboron group, a t-butyldimethylboron group, a triphenylboron group, a phenylboron group, and the like, but are not limited thereto.

In the present specification, the alkyl group may be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. Specific examples of the alkyl group include a methyl group, an ethyl group, a propyl group, an isopropyl group, a butyl group, an isobutyl group, a tert-butyl group, a pentyl group, a hexyl group, a heptyl group, an octyl group, and the like, but are not limited thereto.

In the present specification, an amine group may be selected from the group consisting of —NH₂; an alkylamine group; an N-alkylarylamine group; an arylamine group; an N-arylheteroarylamine group; an N-alkylheteroarylamine group; and a heteroarylamine group, and the number of carbon atoms thereof is not particularly limited, but is preferably 1 to 30. Specific examples of the amine group include a methylamine group; a dimethylamine group; an ethylamine group; a diethylamine group; a phenylamine group; a naphthylamine group; a biphenylamine group; an anthracenylamine group; a 9-methylanthracenylamine group; a diphenylamine group; a ditolylamine group; an N-phenyltolylamine group; a triphenylamine group; an N-phenylbiphenylamine group; an N-phenylnaphthylamine group; an N-biphenylnaphthylamine group; an N-naphthylfluorenylamine group; an N-phenylphenanthrenylamine group; an N-biphenylphenanthrenylamine group; an N-phenylfluorenylamine group; an N-phenyl terphenylamine group; an N-phenanthrenylfluorenylamine group; an N-biphenylfluorenylamine group; and the like, but are not limited thereto.

In the present specification, an N-alkylarylamine group means an amine group in which an alkyl group and an aryl group are substituted with N of the amine group.

In the present specification, an N-arylheteroarylamine group means an amine group in which an aryl group and a heteroaryl group are substituted with N of the amine group.

In the present specification, an N-alkylheteroarylamine group means an amine group in which an alkyl group and a heteroaryl group are substituted with N of the amine group.

In the present specification, the alkyl group in the alkylamine group, the N-arylalkylamine group, the alkylthioxy group, the alkylsulfoxy group, and the N-alkylheteroarylamine group is the same as the above-described examples of the alkyl group. Specifically, examples of the alkylthioxy group include a methylthioxy group; an ethylthioxy group; a tert-butylthioxy group; a hexylthioxy group; an octylthioxy group; and the like, and examples of the alkylsulfoxy group include mesyl; an ethylsulfoxy group; a propylsulfoxy group; a butylsulfoxy group; and the like, but the examples are not limited thereto.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to still another exemplary embodiment, the number of carbon atoms of the cycloalkyl group is 3 to 6. Specific examples thereof include a cyclopropyl group, a cyclobutyl group, a cyclopentyl group, a cyclohexyl group, a cycloheptyl group, a cyclooctyl group, and the like, but are not limited thereto.

In the present specification, an aryl group is not particularly limited, but has preferably 6 to 60 carbon atoms, and may be a monocyclic aryl group or a polycyclic aryl group. According to one embodiment, the number of carbon atoms of the aryl group is from 6 to 30. According to one embodiment, the number of carbon atoms of the aryl group is from 6 to 20. Examples of a monocyclic aryl group as the aryl group include a phenyl group, a biphenyl group, a terphenyl group, and the like, but are not limited thereto. Examples of the multicyclic aryl group include a naphthyl group, an anthracenyl group, a phenanthrenyl group, a pyrenyl group, a perylenyl group, a triphenylene group, a chrysenyl group, a fluorenyl group, and the like, but are not limited thereto.

In the present specification, a heteroaryl group is a cyclic group including one or more of N, O, P, S, Si, and Se as a heteroatom, and the number of carbon atoms thereof is not particularly limited, but is preferably 2 to 60. According to one embodiment, the number of carbon atoms of the heterocyclic group is from 2 to 30. Examples of the heterocyclic group include a pyridine group, a pyrrole group, a pyrimidine group, a pyridazinyl group, a furan group, a thiophene group, an imidazole group, a pyrazole group, a dibenzofuran group, a dibenzothiophene group, a carbazole group, and the like, but are not limited thereto. In the present specification, an arylene group is the same as that defined in the aryl group, except for being a divalent group.

In the present specification, a heteroarylene group is the same as that defined in the heteroaryl group, except for being a divalent group.

In the present specification, a fused ring refers to a ring in which two or more selected from aliphatic hydrocarbon rings, aromatic hydrocarbon rings, and hetero rings are fused together, the definition of the cycloalkyl group is applied to the definition of the aliphatic hydrocarbon ring, except for those that are not monovalent, the definition of the aryl group is applied to the definition of the aromatic hydrocarbon ring, except for those that are not monovalent, and the definition of the heteroaryl group is applied to the definition of the hetero ring, except for those that are not monovalent.

According to an exemplary embodiment of the present specification, Chemical Formula 1 is the following Chemical Formula 1-1 or 1-2.

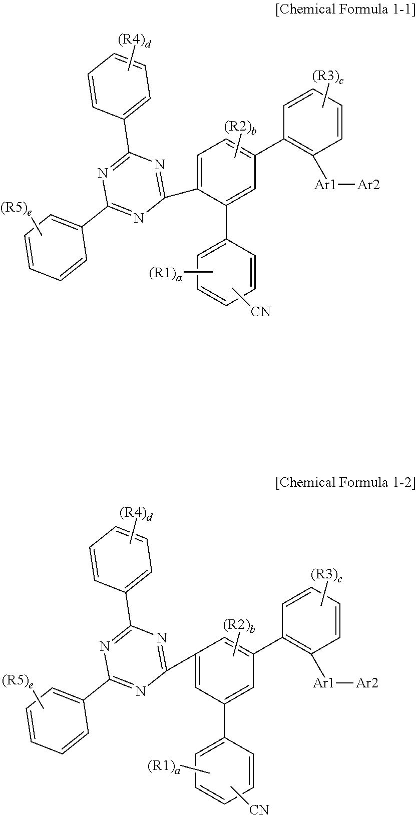

In Chemical Formulae 1-1 and 1-2, R1 to R5, Ar1, Ar2, and a to e are the same as those defined in Chemical Formula

According to an exemplary embodiment of the present specification, Chemical Formula 1 is any one of the following Chemical Formulae 1-3 to 1-7.

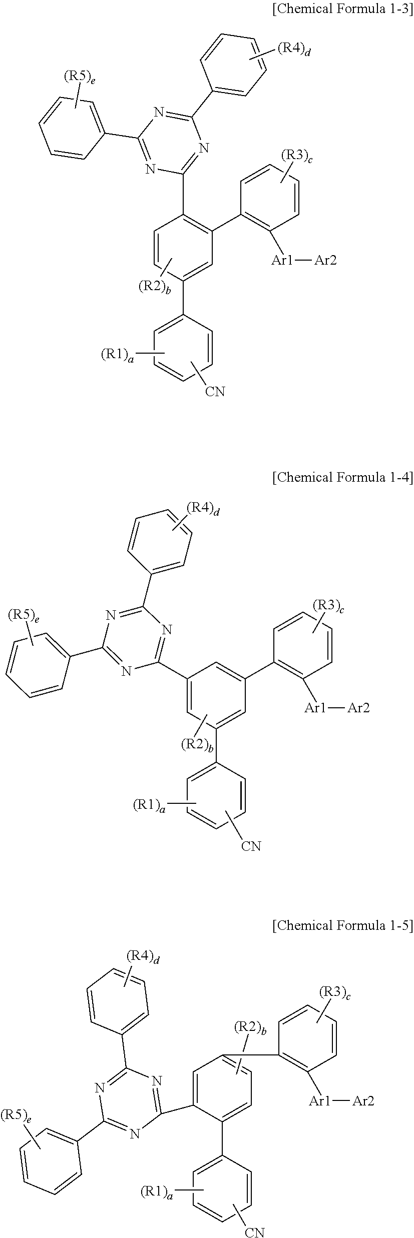

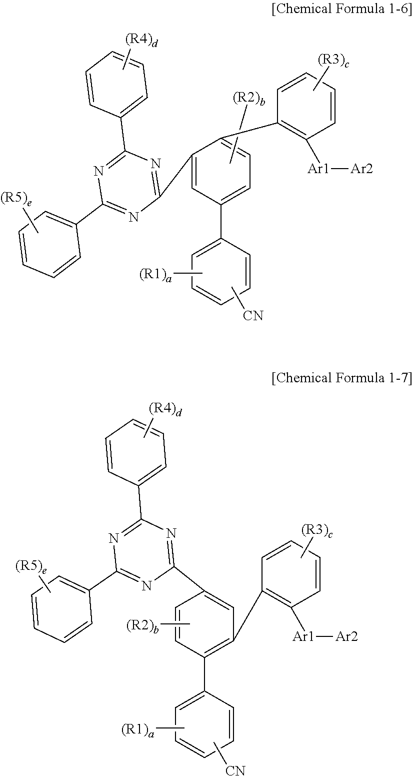

In Chemical Formulae 1-3 to 1-7, R1 to R5, Ar1, Ar2, and a to e are the same as those defined in Chemical Formula 1.

According to an exemplary embodiment of the present specification, Chemical Formula 1 is the following Chemical Formula 1-1-1 or 1-1-2.

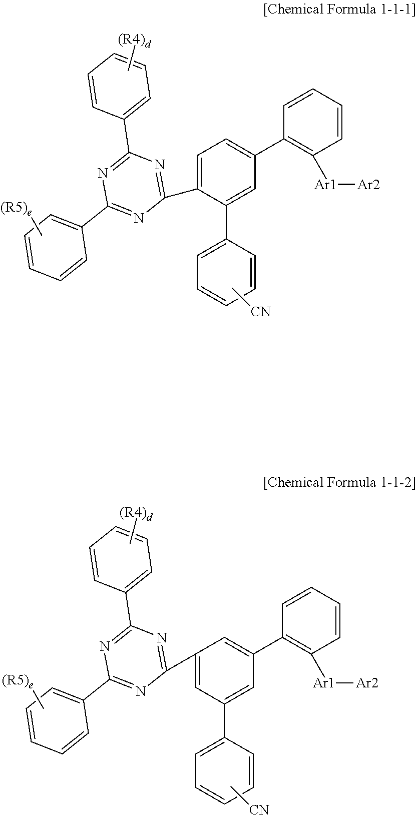

In Chemical Formulae 1-1-1 and 1-1-2, R4, R5, Ar1, Ar2, d, and e are the same as those defined in Chemical Formula

According to an exemplary embodiment of the present specification, Chemical Formula 1 is any one of the following Chemical Formulae 1-1-3 to 1-1-7.

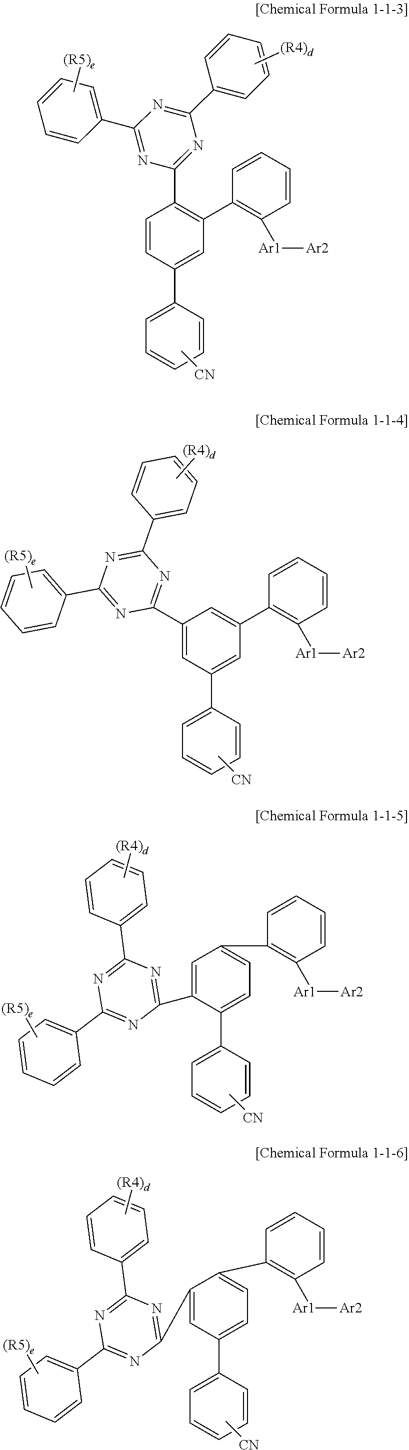

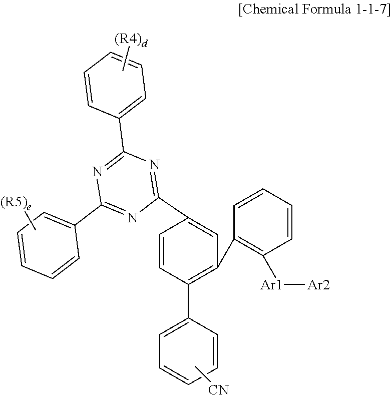

In Chemical Formulae 1-1-3 to 1-1-7, R4, R5, Ar1, Ar2, d, and e are the same as those defined in Chemical Formula 1.

According to an exemplary embodiment of the present specification, Ar1 is a heteroaryl group including two or more N atoms and having 3 to 30 carbon atoms, which is unsubstituted or substituted with an aryl group having 6 to 30 carbon atoms.

According to an exemplary embodiment of the present specification, Ar1 is a heteroaryl group including two or more N atoms and having 3 to 30 carbon atoms, which is unsubstituted or substituted with an aryl group having 6 to 20 carbon atoms.

According to an exemplary embodiment of the present specification, Ar1 is a heteroaryl group including two or more N atoms and having 3 to 30 carbon atoms, and the heteroaryl group is unsubstituted or substituted with a phenyl group, a biphenyl group, a terphenyl group, a naphthyl group, or an anthracene group.

According to an exemplary embodiment of the present specification, Ar1 is a heteroaryl group including two or more N atoms and having 3 to 30 carbon atoms, and the heteroaryl group is unsubstituted or substituted with a phenyl group, a biphenyl group, a terphenyl group, or a naphthyl group.

According to an exemplary embodiment of the present specification, Ar1 is a substituted or unsubstituted heteroaryl group including two or more N atoms and having 3 to 30 carbon atoms.

According to an exemplary embodiment of the present specification, Ar1 is a heteroaryl group including two or more N atoms and having 3 to 30 carbon atoms.

According to an exemplary embodiment of the present specification, Ar1 is a heteroaryl group including two or more N atoms and having 3 to 20 carbon atoms.

According to an exemplary embodiment of the present specification, Ar1 is a heteroaryl group including two or more N atoms and having 3 to 15 carbon atoms.

According to an exemplary embodiment of the present specification, Ar1 is a monocyclic or polycyclic heteroaryl group including two or more N atoms and having 3 to 30 carbon atoms.

According to an exemplary embodiment of the present specification, Ar1 is a monocyclic or polycyclic heteroaryl group including two or more N atoms and having 3 to 20 carbon atoms.

According to an exemplary embodiment of the present specification, Ar1 is a monocyclic or polycyclic heteroaryl group including two or more N atoms and having 3 to 15 carbon atoms.

According to an exemplary embodiment of the present specification, Ar1 is a monocyclic heteroaryl group including two or more N atoms and having 3 to 30 carbon atoms.

According to an exemplary embodiment of the present specification, Ar1 is a monocyclic heteroaryl group including two or more N atoms and having 3 to 20 carbon atoms.

According to an exemplary embodiment of the present specification, Ar1 is a monocyclic heteroaryl group including two or more N atoms and having 3 to 15 carbon atoms.

According to an exemplary embodiment of the present specification, Ar1 is a triazine group or a pyrimidine group, and the triazine group and the pyrimidine group are unsubstituted or substituted with an aryl group having 6 to 20 carbon atoms.

According to an exemplary embodiment of the present specification, Ar1 is a triazine group or a pyrimidine group, and the triazine group and the pyrimidine group are unsubstituted or substituted with a phenyl group, a biphenyl group, or a naphthyl group.

According to an exemplary embodiment of the present specification, Ar1 is a triazine group unsubstituted or substituted with an aryl group having 6 to 20 carbon atoms.

According to an exemplary embodiment of the present specification, Ar1 is a pyrimidine group unsubstituted or substituted with an aryl group having 6 to 20 carbon atoms.

According to an exemplary embodiment of the present specification, Ar1 is a triazine group unsubstituted or substituted with a phenyl group, a biphenyl group, or a naphthyl group.

According to an exemplary embodiment of the present specification, Ar1 is a pyrimidine group unsubstituted or substituted with a phenyl group, a biphenyl group, or a naphthyl group.

According to an exemplary embodiment of the present specification, Ar1 is a triazine group or a pyrimidine group.

According to an exemplary embodiment of the present specification, Ar1 is a triazine group.

According to an exemplary embodiment of the present specification, Ar1 is a pyrimidine group.

According to an exemplary embodiment of the present specification, Ar1 is a polycyclic heteroaryl group including two or more N atoms and having 3 to 20 carbon atoms.

According to an exemplary embodiment of the present specification, Ar1 is a polycyclic heteroaryl group including two or more N atoms and having 3 to 15 carbon atoms.

According to an exemplary embodiment of the present specification, Ar1 is a quinazoline group.

According to an exemplary embodiment of the present specification, Ar1 is a quinoxaline group.

According to an exemplary embodiment of the present specification, Ar2 is hydrogen, deuterium, a nitrile group, a halogen group, a substituted or unsubstituted alkyl group having 1 to 10 carbon atoms, a substituted or unsubstituted aryl group having 6 to 30 carbon atoms, a substituted or unsubstituted heteroaryl group having 3 to 30 carbon atoms, or a substituted or unsubstituted fused ring group having 3 to 30 carbon atoms.

According to an exemplary embodiment of the present specification, Ar2 is a substituted or unsubstituted alkyl group having 1 to 10 carbon atoms, or a substituted or unsubstituted aryl group having 6 to 30 carbon atoms.

According to an exemplary embodiment of the present specification, Ar2 is a substituted or unsubstituted alkyl group having 1 to 6 carbon atoms, or a substituted or unsubstituted aryl group having 6 to 20 carbon atoms.

According to an exemplary embodiment of the present specification, Ar2 is a substituted or unsubstituted alkyl group having 1 to 6 carbon atoms, or a substituted or unsubstituted aryl group having 6 to 15 carbon atoms.

According to an exemplary embodiment of the present specification, Ar2 is an aryl group having 6 to 30 carbon atoms.

According to an exemplary embodiment of the present specification, Ar2 is an aryl group having 6 to 20 carbon atoms.

According to an exemplary embodiment of the present specification, Ar2 is an aryl group having 6 to 15 carbon atoms.

According to an exemplary embodiment of the present specification, Ar2 is a methyl group, an ethyl group, a tert-butyl group, an isopropyl group, a phenyl group, a biphenyl group, a naphthyl group, an anthracene group, or a phenanthrene group.

According to an exemplary embodiment of the present specification, Ar2 is a methyl group, an ethyl group, a phenyl group, a biphenyl group, or a naphthyl group.

According to an exemplary embodiment of the present specification, R1 to R5 are the same as or different from each other, and are each independently hydrogen, deuterium, a nitrile group, a halogen group, a substituted or unsubstituted alkyl group, a substituted or unsubstituted aryl group, or a substituted or unsubstituted heteroaryl group.

According to an exemplary embodiment of the present specification, R1 to R5 are the same as or different from each other, and are each independently hydrogen, deuterium, a nitrile group, a halogen group, a substituted or unsubstituted alkyl group having 1 to 10 carbon atoms, a substituted or unsubstituted aryl group having 6 to 30 carbon atoms, or a substituted or unsubstituted heteroaryl group having 3 to 30 carbon atoms.

According to an exemplary embodiment of the present specification, R1 to R5 are the same as or different from each other, and are each independently hydrogen, deuterium, a nitrile group, a halogen group, or a substituted or unsubstituted alkyl group having 1 to 10 carbon atoms.

According to an exemplary embodiment of the present specification, R1 to R5 are the same as or different from each other, and are each independently hydrogen, deuterium, a nitrile group, a halogen group, an alkyl group having 1 to 10 carbon atoms, an aryl group having 6 to 30 carbon atoms, or a heteroaryl group having 3 to 30 carbon atoms.

According to an exemplary embodiment of the present specification, R1 to R5 are the same as or different from each other, and are each independently hydrogen, deuterium, a nitrile group, a halogen group, or an alkyl group having 1 to 10 carbon atoms.

According to an exemplary embodiment of the present specification, R1 to R5 are the same as or different from each other, and are each independently hydrogen, deuterium, a nitrile group, F, Cl, Br, I, a methyl group, an ethyl group, a propyl group, an isopropyl group, a butyl group, a tert-butyl group, a phenyl group, a naphthyl group, a biphenyl group, a terphenyl group, a pyrrole group, a furan group, a thiophene group, a triazine group, a pyrimidine group, a pyridine group, a carbazole group, a dibenzofuran group, or a dibenzothiophene group.

According to an exemplary embodiment of the present specification, R1 to R5 are the same as or different from each other, and are each independently hydrogen, deuterium, a nitrile group, F, Cl, Br, I, a methyl group, an ethyl group, a propyl group, an isopropyl group, a butyl group, a phenyl group, a naphthyl group, or a tert-butyl group.

According to an exemplary embodiment of the present specification, R1 to R5 are the same as or different from each other, and are each independently hydrogen, deuterium, a nitrile group, a halogen group, a phenyl group, or a naphthyl group.

According to an exemplary embodiment of the present specification, R1 to R5 are the same as or different from each other, and are each independently hydrogen, deuterium, a phenyl group, or a naphthyl group.

According to an exemplary embodiment of the present specification, R1 to R5 are hydrogen or deuterium.

According to an exemplary embodiment of the present specification, the compound of Chemical Formula 1 is any one of the following compounds.

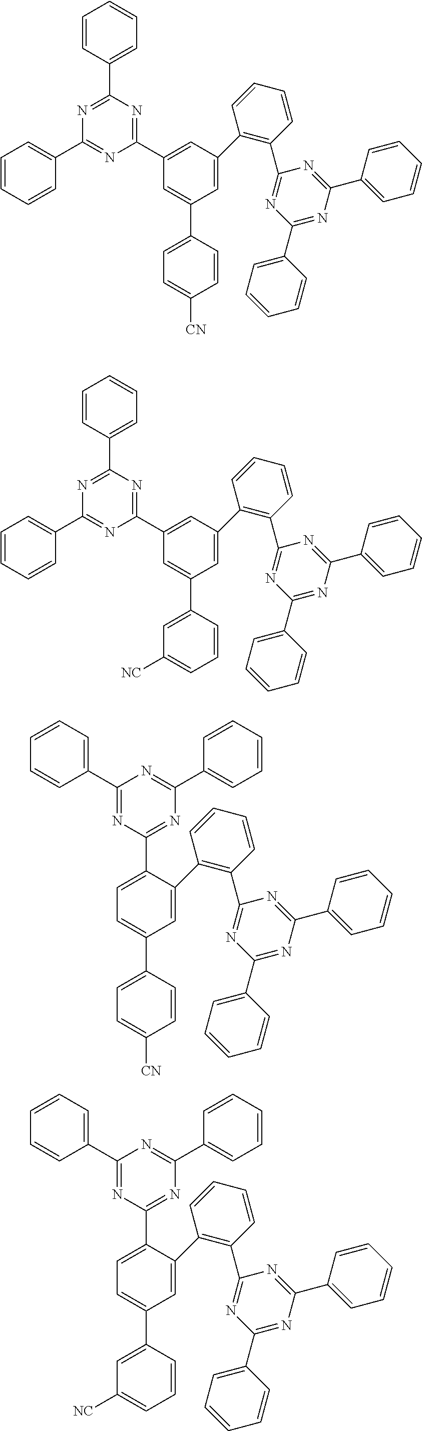

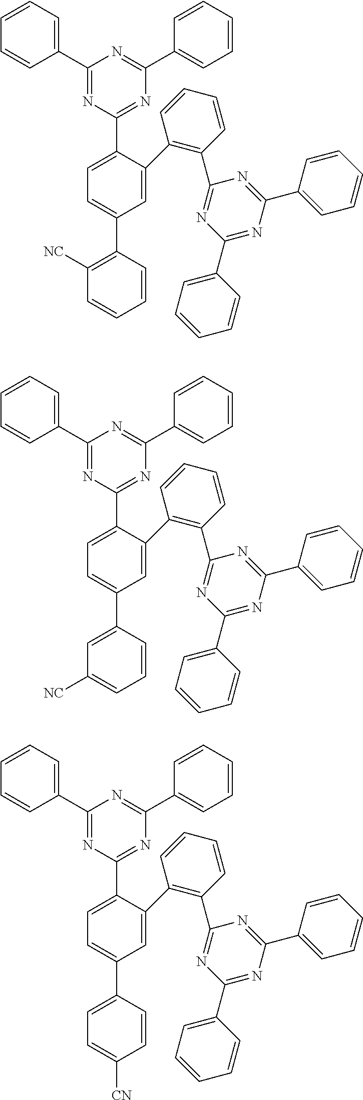

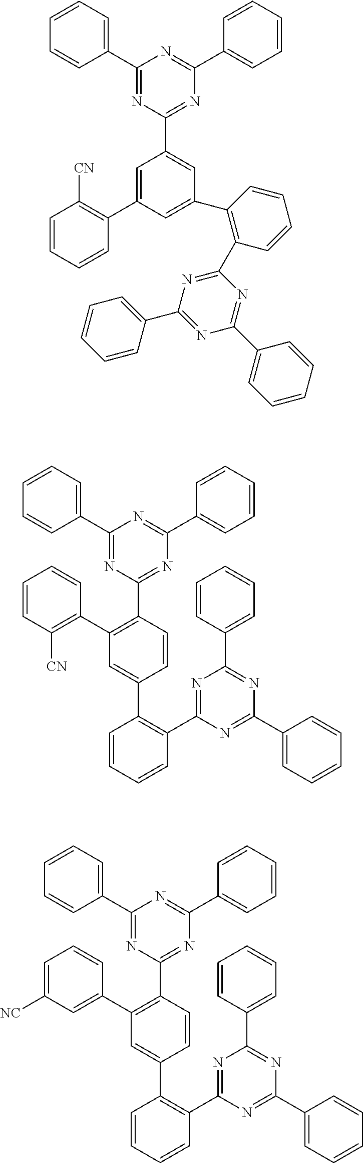 [SMILES: N#Cc1ccccc1-c1cc(-c2nc(-c3ccccc3)nc(-c3ccccc3)n2)cc(-c2ccccc2-c2nc(-c3ccccc3)nc(-c3ccccc3)n2)c1 ; N#Cc1ccccc1-c1cc(-c2ccccc2-c2nc(-c3ccccc3)nc(-c3ccccc3)n2)ccc1-c1nc(-c2ccccc2)nc(-c2ccccc2)n1 ; N#Cc1cccc(-c2cc(-c3ccccc3-c3nc(-c4ccccc4)nc(-c4ccccc4)n3)ccc2-c2nc(-c3ccccc3)nc(-c3ccccc3)n2)c1]

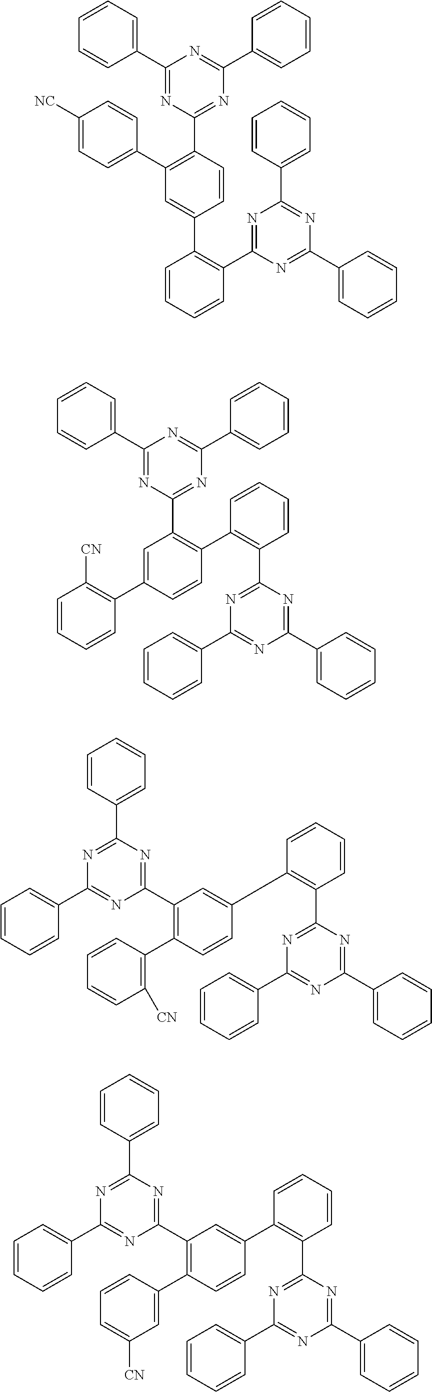 [SMILES: N#Cc1ccc(-c2cc(-c3ccccc3-c3nc(-c4ccccc4)nc(-c4ccccc4)n3)ccc2-c2nc(-c3ccccc3)nc(-c3ccccc3)n2)cc1 ; N#Cc1ccccc1-c1ccc(-c2ccccc2-c2nc(-c3ccccc3)nc(-c3ccccc3)n2)c(-c2nc(-c3ccccc3)nc(-c3ccccc3)n2)c1 ; N#Cc1ccccc1-c1ccc(-c2ccccc2-c2nc(-c3ccccc3)nc(-c3ccccc3)n2)cc1-c1nc(-c2ccccc2)nc(-c2ccccc2)n1 ; N#Cc1cccc(-c2ccc(-c3ccccc3-c3nc(-c4ccccc4)nc(-c4ccccc4)n3)cc2-c2nc(-c3ccccc3)nc(-c3ccccc3)n2)c1]

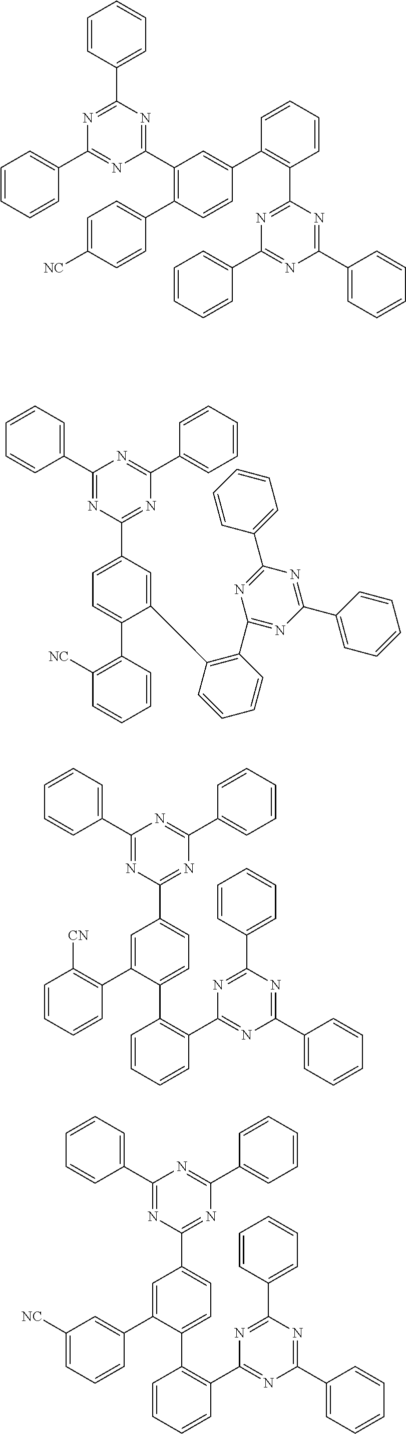 [SMILES: N#Cc1ccc(-c2ccc(-c3ccccc3-c3nc(-c4ccccc4)nc(-c4ccccc4)n3)cc2-c2nc(-c3ccccc3)nc(-c3ccccc3)n2)cc1 ; N#Cc1ccccc1-c1ccc(-c2nc(-c3ccccc3)nc(-c3ccccc3)n2)cc1-c1ccccc1-c1nc(-c2ccccc2)nc(-c2ccccc2)n1 ; N#Cc1ccccc1-c1cc(-c2nc(-c3ccccc3)nc(-c3ccccc3)n2)ccc1-c1ccccc1-c1nc(-c2ccccc2)nc(-c2ccccc2)n1 ; N#Cc1cccc(-c2cc(-c3nc(-c4ccccc4)nc(-c4ccccc4)n3)ccc2-c2ccccc2-c2nc(-c3ccccc3)nc(-c3ccccc3)n2)c1]

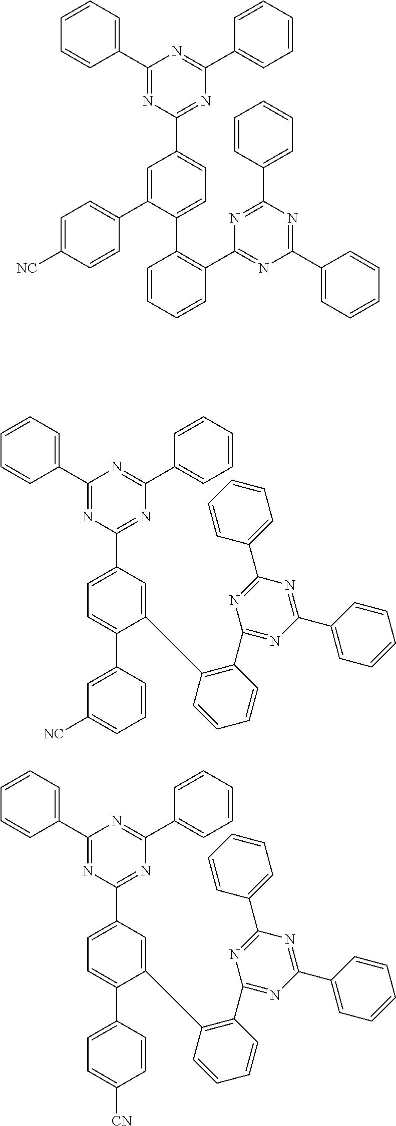

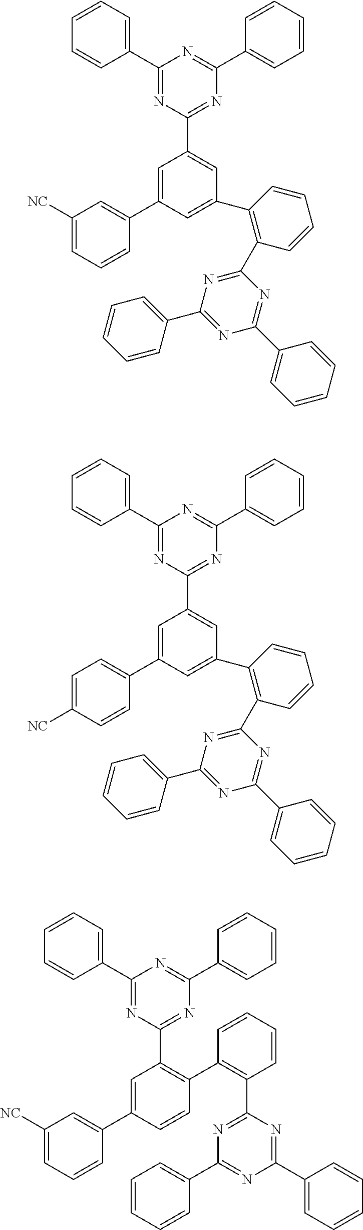 [SMILES: N#Cc1cccc(-c2cc(-c3nc(-c4ccccc4)nc(-c4ccccc4)n3)cc(-c3ccccc3-c3nc(-c4ccccc4)nc(-c4ccccc4)n3)c2)c1 ; N#Cc1ccc(-c2cc(-c3nc(-c4ccccc4)nc(-c4ccccc4)n3)cc(-c3ccccc3-c3nc(-c4ccccc4)nc(-c4ccccc4)n3)c2)cc1 ; N#Cc1cccc(-c2ccc(-c3ccccc3-c3nc(-c4ccccc4)nc(-c4ccccc4)n3)c(-c3nc(-c4ccccc4)nc(-c4ccccc4)n3)c2)c1]

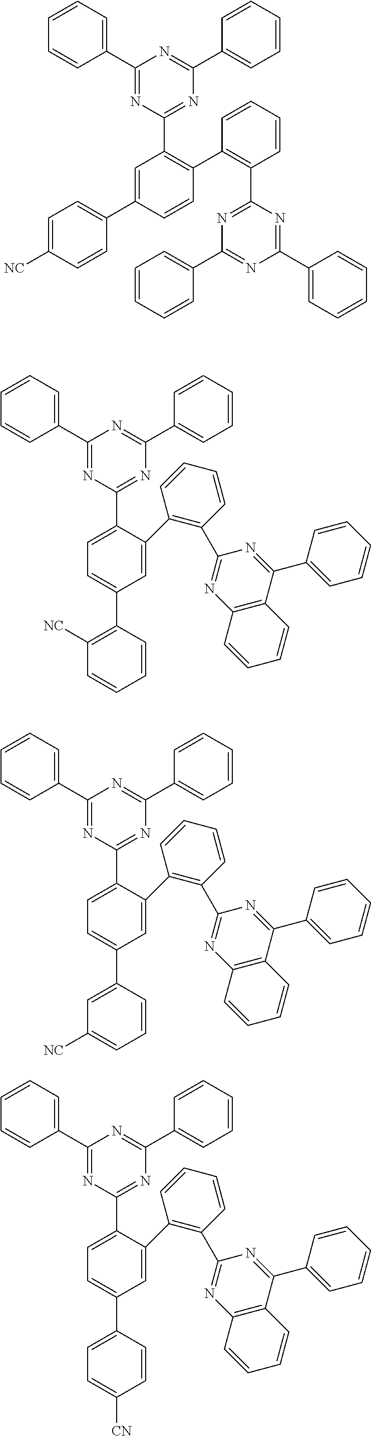

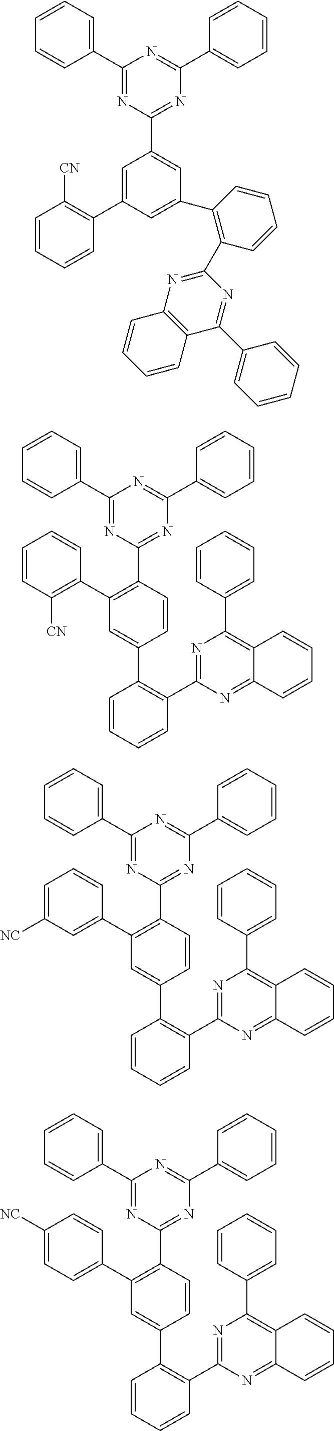

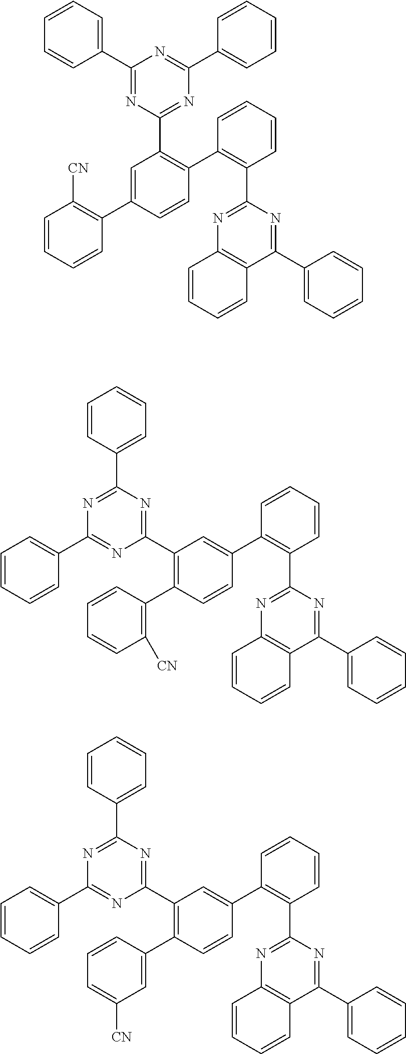 [SMILES: N#Cc1ccccc1-c1ccc(-c2ccccc2-c2nc(-c3ccccc3)c3ccccc3n2)c(-c2nc(-c3ccccc3)nc(-c3ccccc3)n2)c1 ; N#Cc1ccccc1-c1ccc(-c2ccccc2-c2nc(-c3ccccc3)c3ccccc3n2)cc1-c1nc(-c2ccccc2)nc(-c2ccccc2)n1 ; N#Cc1cccc(-c2ccc(-c3ccccc3-c3nc(-c4ccccc4)c4ccccc4n3)cc2-c2nc(-c3ccccc3)nc(-c3ccccc3)n2)c1]

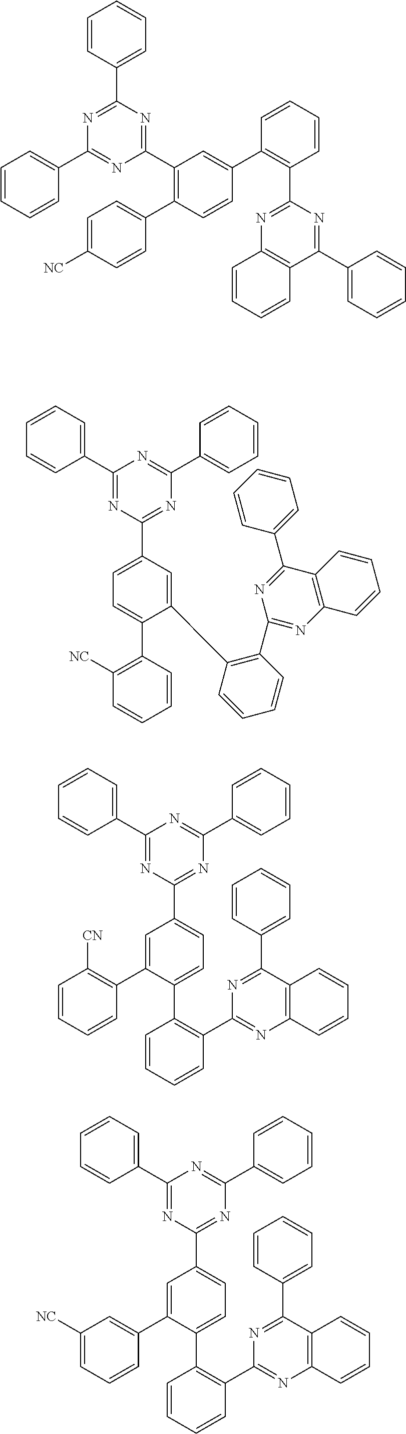 [SMILES: N#Cc1ccc(-c2ccc(-c3ccccc3-c3nc(-c4ccccc4)c4ccccc4n3)cc2-c2nc(-c3ccccc3)nc(-c3ccccc3)n2)cc1 ; N#Cc1ccccc1-c1ccc(-c2nc(-c3ccccc3)nc(-c3ccccc3)n2)cc1-c1ccccc1-c1nc(-c2ccccc2)c2ccccc2n1 ; N#Cc1ccccc1-c1cc(-c2nc(-c3ccccc3)nc(-c3ccccc3)n2)ccc1-c1ccccc1-c1nc(-c2ccccc2)c2ccccc2n1 ; N#Cc1cccc(-c2cc(-c3nc(-c4ccccc4)nc(-c4ccccc4)n3)ccc2-c2ccccc2-c2nc(-c3ccccc3)c3ccccc3n2)c1]

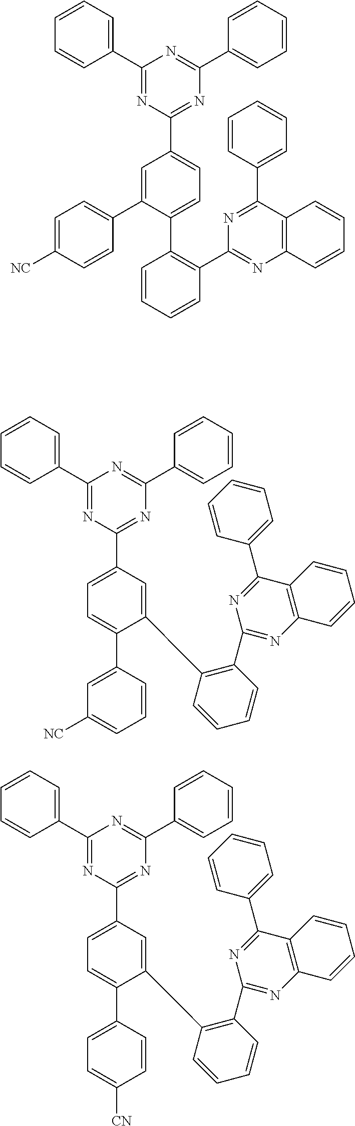

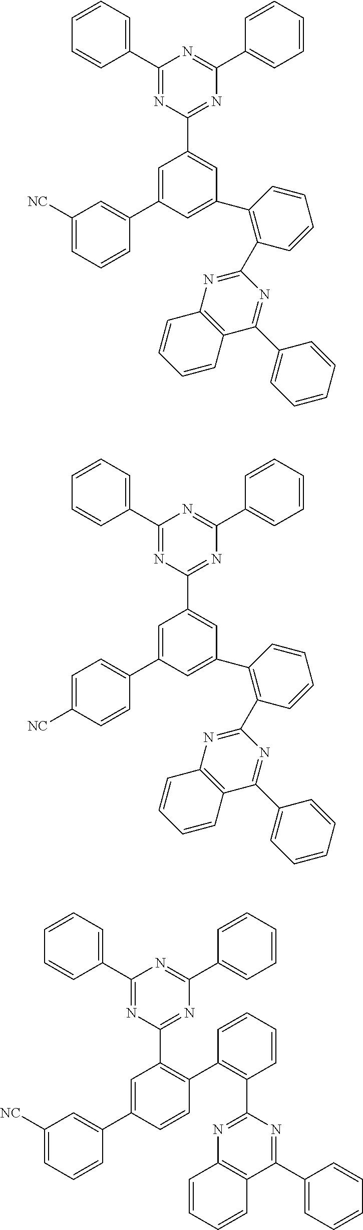 [SMILES: N#Cc1cccc(-c2cc(-c3nc(-c4ccccc4)nc(-c4ccccc4)n3)cc(-c3ccccc3-c3nc(-c4ccccc4)c4ccccc4n3)c2)c1 ; N#Cc1ccc(-c2cc(-c3nc(-c4ccccc4)nc(-c4ccccc4)n3)cc(-c3ccccc3-c3nc(-c4ccccc4)c4ccccc4n3)c2)cc1 ; N#Cc1cccc(-c2ccc(-c3ccccc3-c3nc(-c4ccccc4)c4ccccc4n3)c(-c3nc(-c4ccccc4)nc(-c4ccccc4)n3)c2)c1]

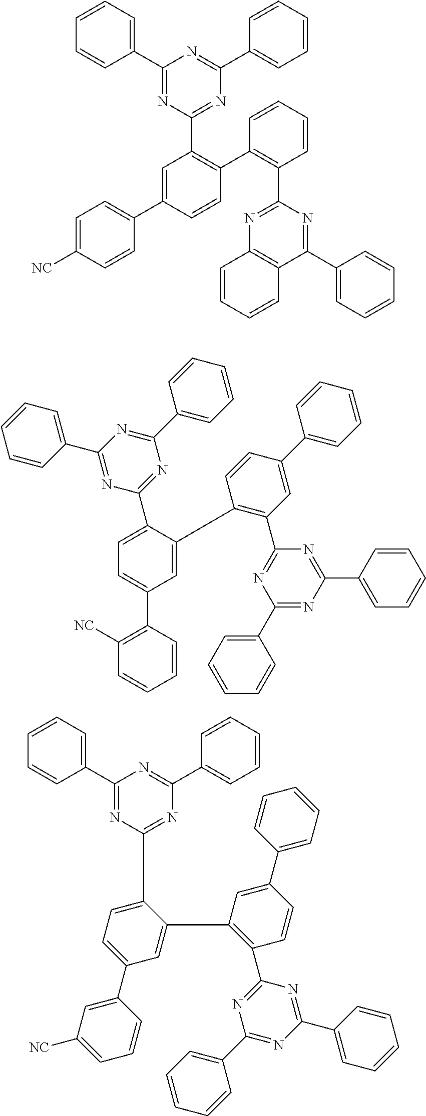

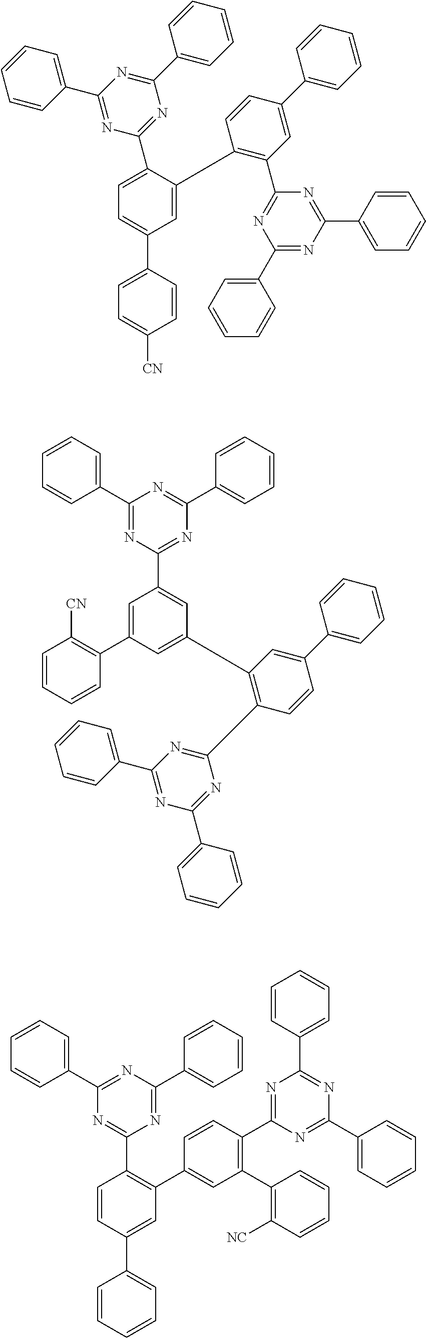 [SMILES: N#Cc1ccc(-c2ccc(-c3nc(-c4ccccc4)nc(-c4ccccc4)n3)c(-c3ccc(-c4ccccc4)cc3-c3nc(-c4ccccc4)nc(-c4ccccc4)n3)c2)cc1 ; N#Cc1ccccc1-c1cc(-c2nc(-c3ccccc3)nc(-c3ccccc3)n2)cc(-c2cc(-c3ccccc3)ccc2-c2nc(-c3ccccc3)nc(-c3ccccc3)n2)c1 ; N#Cc1ccccc1-c1cc(-c2cc(-c3ccccc3)ccc2-c2nc(-c3ccccc3)nc(-c3ccccc3)n2)ccc1-c1nc(-c2ccccc2)nc(-c2ccccc2)n1]

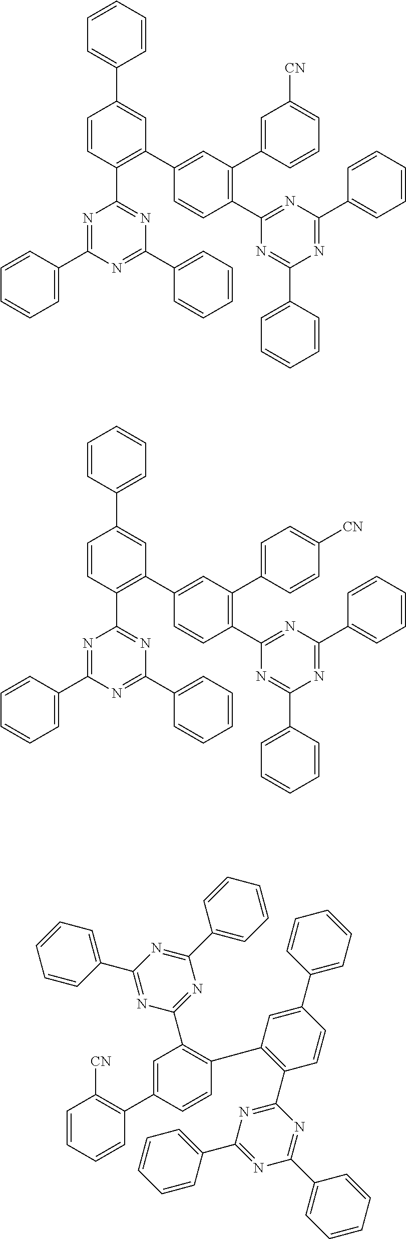 [SMILES: N#Cc1cccc(-c2cc(-c3cc(-c4ccccc4)ccc3-c3nc(-c4ccccc4)nc(-c4ccccc4)n3)ccc2-c2nc(-c3ccccc3)nc(-c3ccccc3)n2)c1 ; N#Cc1ccc(-c2cc(-c3cc(-c4ccccc4)ccc3-c3nc(-c4ccccc4)nc(-c4ccccc4)n3)ccc2-c2nc(-c3ccccc3)nc(-c3ccccc3)n2)cc1 ; N#Cc1ccccc1-c1ccc(-c2cc(-c3ccccc3)ccc2-c2nc(-c3ccccc3)nc(-c3ccccc3)n2)c(-c2nc(-c3ccccc3)nc(-c3ccccc3)n2)c1]

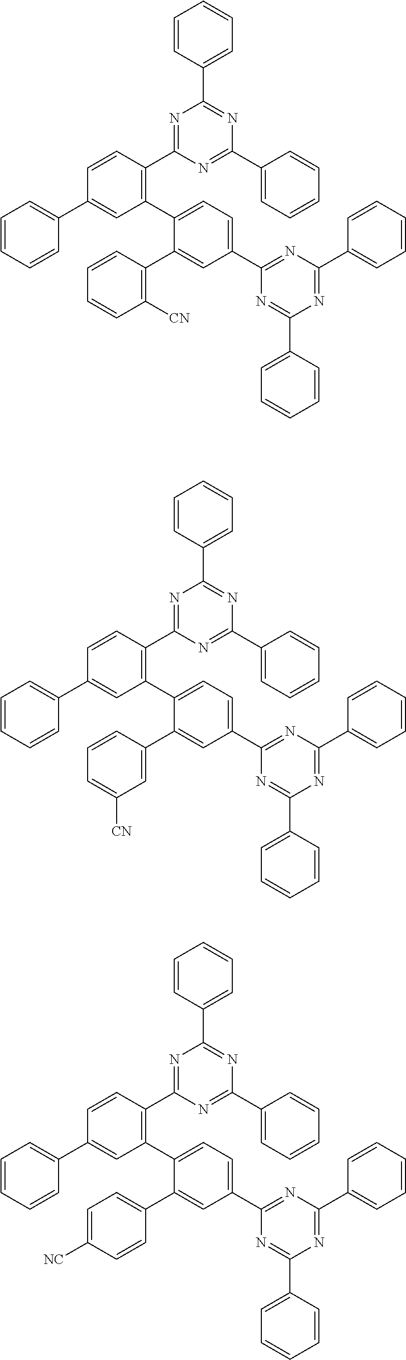 [SMILES: N#Cc1ccccc1-c1cc(-c2nc(-c3ccccc3)nc(-c3ccccc3)n2)ccc1-c1cc(-c2ccccc2)ccc1-c1nc(-c2ccccc2)nc(-c2ccccc2)n1 ; N#Cc1cccc(-c2cc(-c3nc(-c4ccccc4)nc(-c4ccccc4)n3)ccc2-c2cc(-c3ccccc3)ccc2-c2nc(-c3ccccc3)nc(-c3ccccc3)n2)c1 ; N#Cc1ccc(-c2cc(-c3nc(-c4ccccc4)nc(-c4ccccc4)n3)ccc2-c2cc(-c3ccccc3)ccc2-c2nc(-c3ccccc3)nc(-c3ccccc3)n2)cc1]

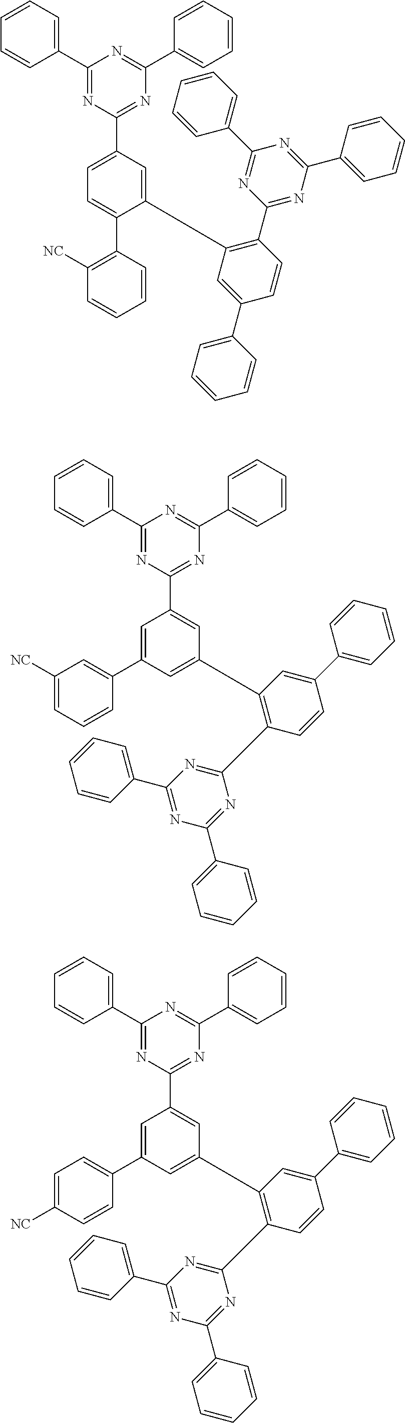

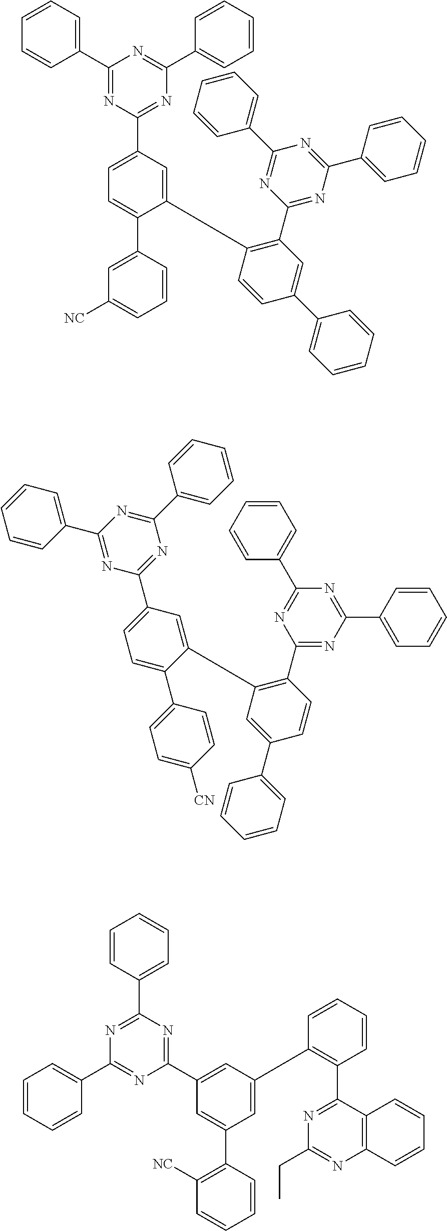 [SMILES: N#Cc1cccc(-c2ccc(-c3nc(-c4ccccc4)nc(-c4ccccc4)n3)cc2-c2ccc(-c3ccccc3)cc2-c2nc(-c3ccccc3)nc(-c3ccccc3)n2)c1 ; N#Cc1ccc(-c2ccc(-c3nc(-c4ccccc4)nc(-c4ccccc4)n3)cc2-c2cc(-c3ccccc3)ccc2-c2nc(-c3ccccc3)nc(-c3ccccc3)n2)cc1 ; CCc1nc(-c2ccccc2-c2cc(-c3nc(-c4ccccc4)nc(-c4ccccc4)n3)cc(-c3ccccc3C#N)c2)c2ccccc2n1]

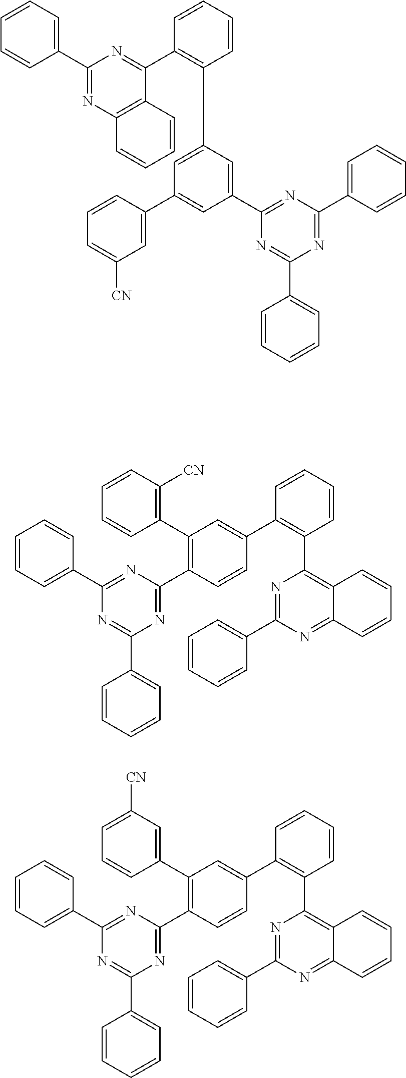

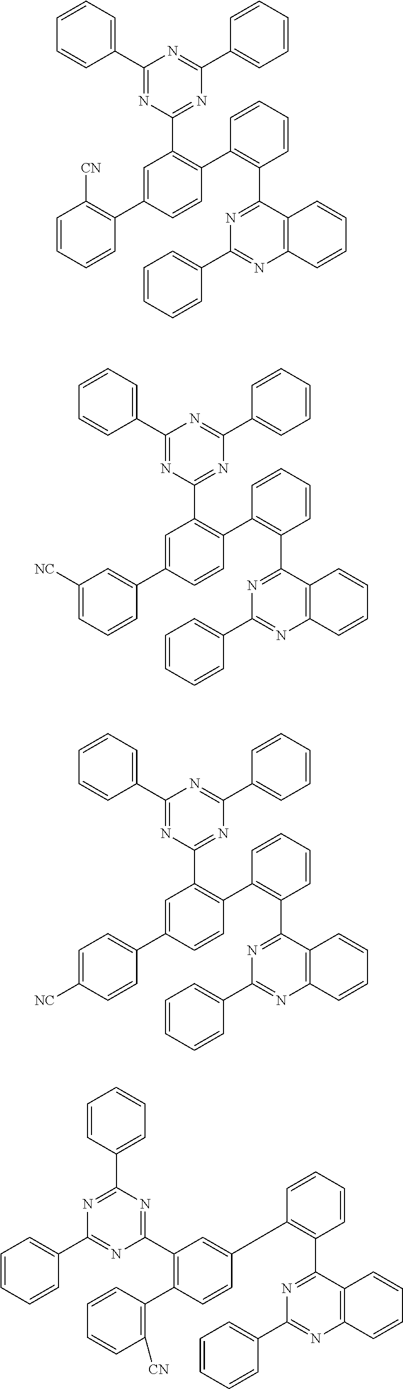 [SMILES: N#Cc1ccccc1-c1ccc(-c2ccccc2-c2nc(-c3ccccc3)nc3ccccc23)c(-c2nc(-c3ccccc3)nc(-c3ccccc3)n2)c1 ; N#Cc1cccc(-c2ccc(-c3ccccc3-c3nc(-c4ccccc4)nc4ccccc34)c(-c3nc(-c4ccccc4)nc(-c4ccccc4)n3)c2)c1 ; N#Cc1ccc(-c2ccc(-c3ccccc3-c3nc(-c4ccccc4)nc4ccccc34)c(-c3nc(-c4ccccc4)nc(-c4ccccc4)n3)c2)cc1 ; N#Cc1ccccc1-c1ccc(-c2ccccc2-c2nc(-c3ccccc3)nc3ccccc23)cc1-c1nc(-c2ccccc2)nc(-c2ccccc2)n1]

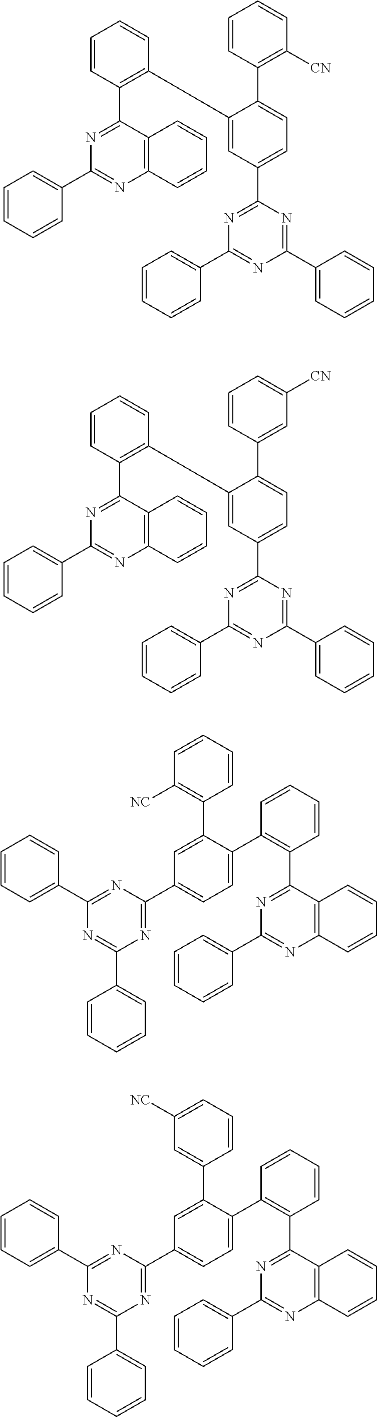 [SMILES: N#Cc1ccccc1-c1ccc(-c2nc(-c3ccccc3)nc(-c3ccccc3)n2)cc1-c1ccccc1-c1nc(-c2ccccc2)nc2ccccc12 ; N#Cc1cccc(-c2ccc(-c3nc(-c4ccccc4)nc(-c4ccccc4)n3)cc2-c2ccccc2-c2nc(-c3ccccc3)nc3ccccc23)c1 ; N#Cc1ccccc1-c1cc(-c2nc(-c3ccccc3)nc(-c3ccccc3)n2)ccc1-c1ccccc1-c1nc(-c2ccccc2)nc2ccccc12 ; N#Cc1cccc(-c2cc(-c3nc(-c4ccccc4)nc(-c4ccccc4)n3)ccc2-c2ccccc2-c2nc(-c3ccccc3)nc3ccccc23)c1]

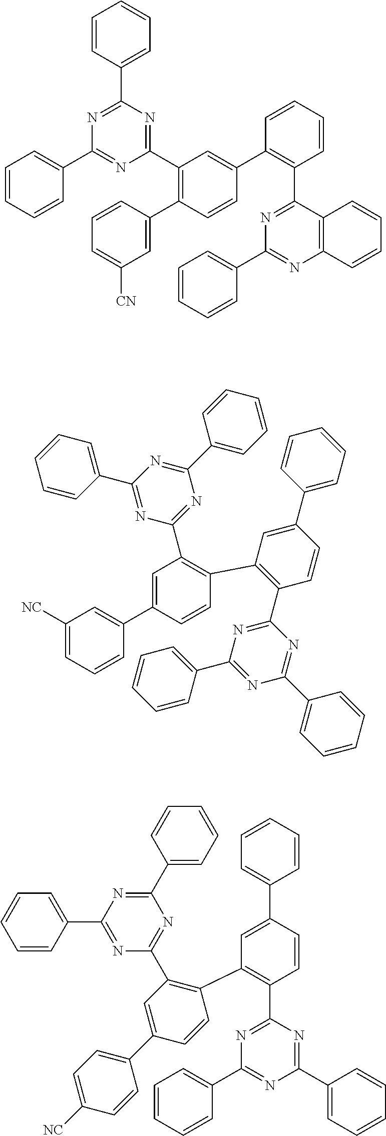 [SMILES: N#Cc1cccc(-c2ccc(-c3ccccc3-c3nc(-c4ccccc4)nc4ccccc34)cc2-c2nc(-c3ccccc3)nc(-c3ccccc3)n2)c1 ; N#Cc1cccc(-c2ccc(-c3cc(-c4ccccc4)ccc3-c3nc(-c4ccccc4)nc(-c4ccccc4)n3)c(-c3nc(-c4ccccc4)nc(-c4ccccc4)n3)c2)c1 ; N#Cc1ccc(-c2ccc(-c3cc(-c4ccccc4)ccc3-c3nc(-c4ccccc4)nc(-c4ccccc4)n3)c(-c3nc(-c4ccccc4)nc(-c4ccccc4)n3)c2)cc1]

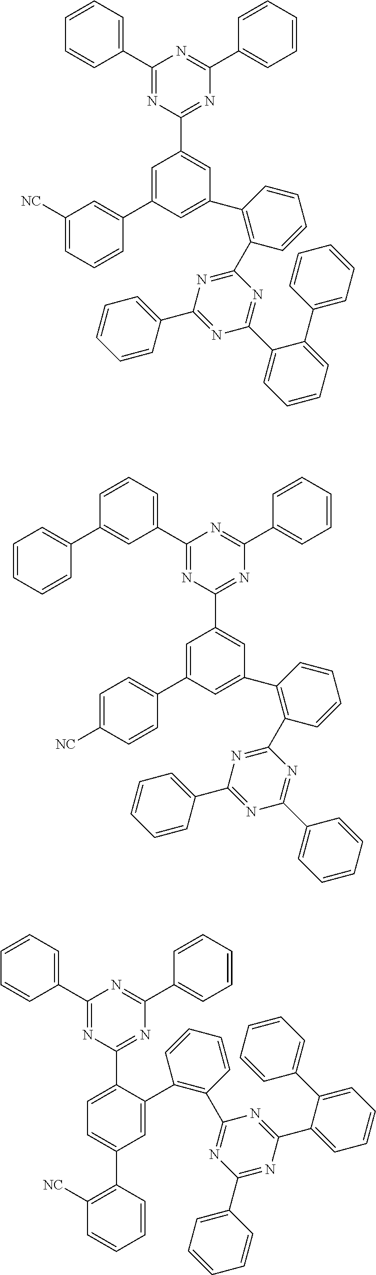 [SMILES: N#Cc1cccc(-c2cc(-c3nc(-c4ccccc4)nc(-c4ccccc4)n3)cc(-c3ccccc3-c3nc(-c4ccccc4)nc(-c4ccccc4-c4ccccc4)n3)c2)c1 ; N#Cc1ccc(-c2cc(-c3nc(-c4ccccc4)nc(-c4cccc(-c5ccccc5)c4)n3)cc(-c3ccccc3-c3nc(-c4ccccc4)nc(-c4ccccc4)n3)c2)cc1 ; N#Cc1ccccc1-c1ccc(-c2nc(-c3ccccc3)nc(-c3ccccc3)n2)c(-c2ccccc2-c2nc(-c3ccccc3)nc(-c3ccccc3-c3ccccc3)n2)c1]

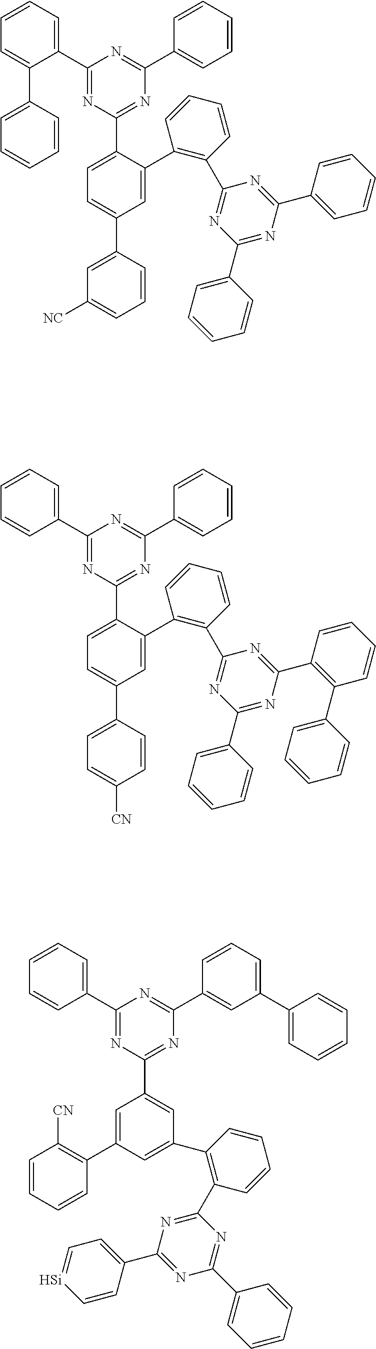

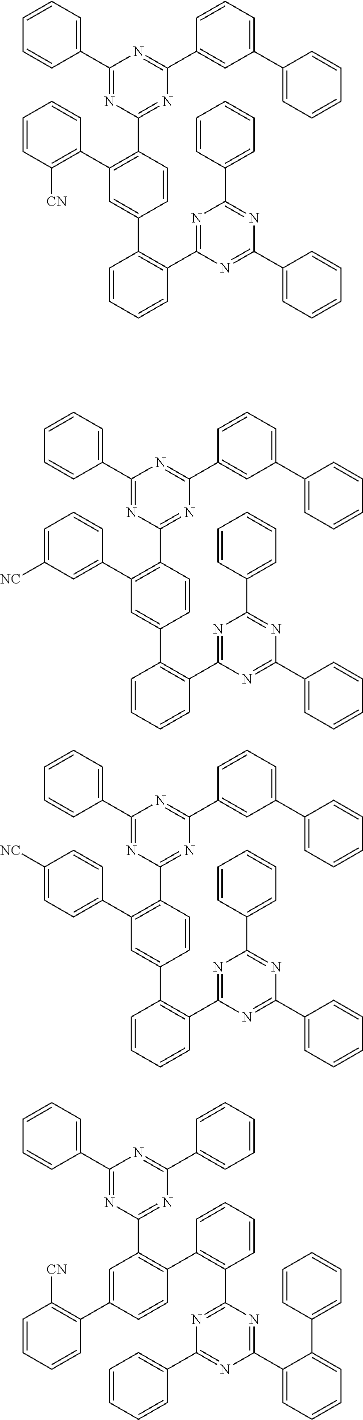 [SMILES: N#Cc1ccccc1-c1cc(-c2ccccc2-c2nc(-c3ccccc3)nc(-c3ccccc3)n2)ccc1-c1nc(-c2ccccc2)nc(-c2cccc(-c3ccccc3)c2)n1 ; N#Cc1cccc(-c2cc(-c3ccccc3-c3nc(-c4ccccc4)nc(-c4ccccc4)n3)ccc2-c2nc(-c3ccccc3)nc(-c3cccc(-c4ccccc4)c3)n2)c1 ; N#Cc1ccc(-c2cc(-c3ccccc3-c3nc(-c4ccccc4)nc(-c4ccccc4)n3)ccc2-c2nc(-c3ccccc3)nc(-c3cccc(-c4ccccc4)c3)n2)cc1 ; N#Cc1ccccc1-c1ccc(-c2ccccc2-c2nc(-c3ccccc3)nc(-c3ccccc3-c3ccccc3)n2)c(-c2nc(-c3ccccc3)nc(-c3ccccc3)n2)c1]

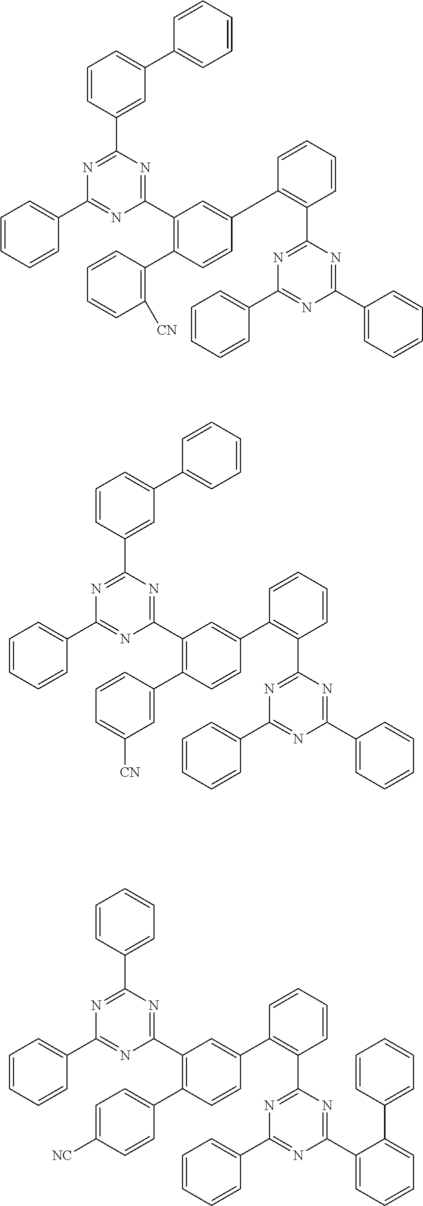 [SMILES: N#Cc1ccccc1-c1ccc(-c2ccccc2-c2nc(-c3ccccc3)nc(-c3ccccc3)n2)cc1-c1nc(-c2ccccc2)nc(-c2cccc(-c3ccccc3)c2)n1 ; N#Cc1cccc(-c2ccc(-c3ccccc3-c3nc(-c4ccccc4)nc(-c4ccccc4)n3)cc2-c2nc(-c3ccccc3)nc(-c3cccc(-c4ccccc4)c3)n2)c1 ; N#Cc1ccc(-c2ccc(-c3ccccc3-c3nc(-c4ccccc4)nc(-c4ccccc4-c4ccccc4)n3)cc2-c2nc(-c3ccccc3)nc(-c3ccccc3)n2)cc1]

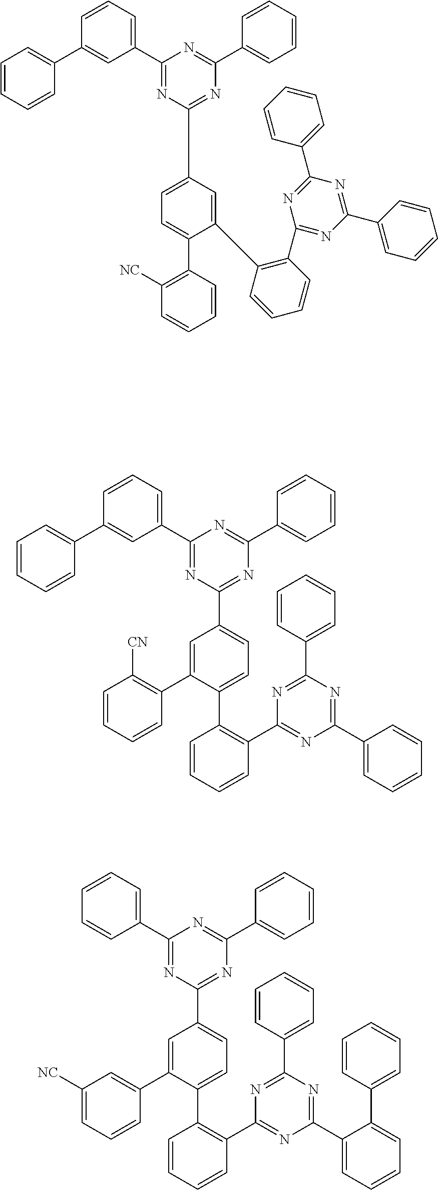 [SMILES: N#Cc1ccccc1-c1ccc(-c2nc(-c3ccccc3)nc(-c3cccc(-c4ccccc4)c3)n2)cc1-c1ccccc1-c1nc(-c2ccccc2)nc(-c2ccccc2)n1 ; N#Cc1ccccc1-c1cc(-c2nc(-c3ccccc3)nc(-c3cccc(-c4ccccc4)c3)n2)ccc1-c1ccccc1-c1nc(-c2ccccc2)nc(-c2ccccc2)n1 ; N#Cc1cccc(-c2cc(-c3nc(-c4ccccc4)nc(-c4ccccc4)n3)ccc2-c2ccccc2-c2nc(-c3ccccc3)nc(-c3ccccc3-c3ccccc3)n2)c1]

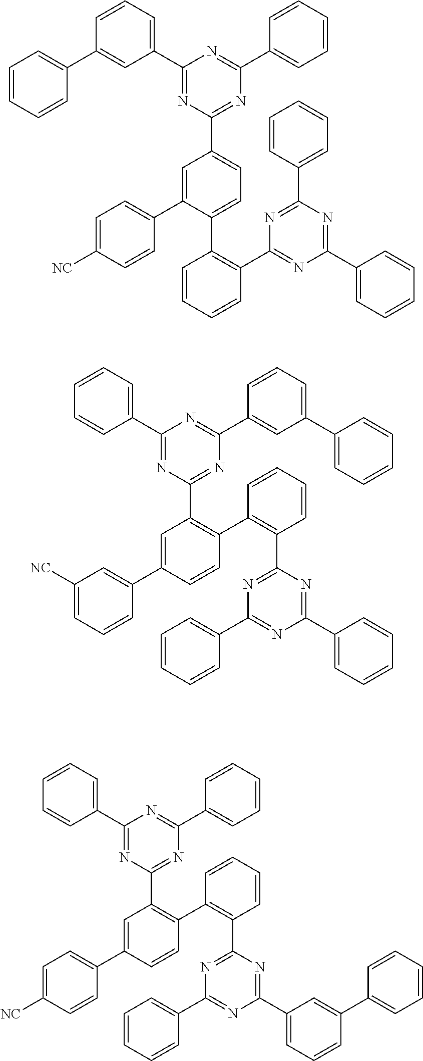 [SMILES: N#Cc1ccc(-c2cc(-c3nc(-c4ccccc4)nc(-c4cccc(-c5ccccc5)c4)n3)ccc2-c2ccccc2-c2nc(-c3ccccc3)nc(-c3ccccc3)n2)cc1 ; N#Cc1cccc(-c2ccc(-c3ccccc3-c3nc(-c4ccccc4)nc(-c4ccccc4)n3)c(-c3nc(-c4ccccc4)nc(-c4cccc(-c5ccccc5)c4)n3)c2)c1 ; N#Cc1ccc(-c2ccc(-c3ccccc3-c3nc(-c4ccccc4)nc(-c4cccc(-c5ccccc5)c4)n3)c(-c3nc(-c4ccccc4)nc(-c4ccccc4)n3)c2)cc1]

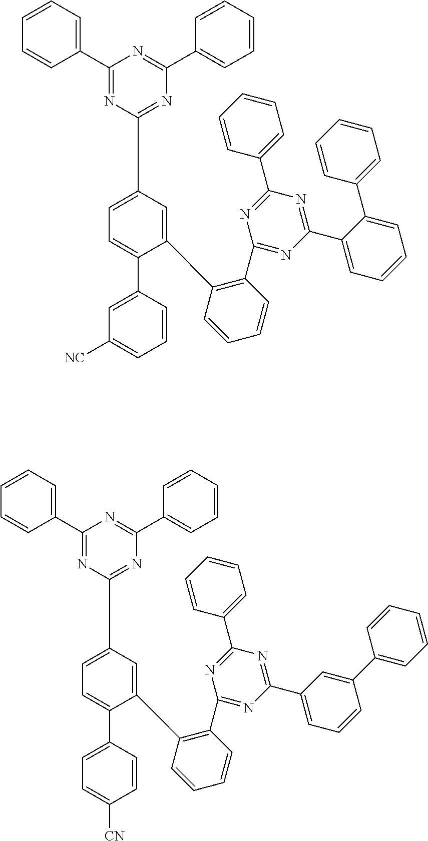 [SMILES: N#Cc1cccc(-c2ccc(-c3nc(-c4ccccc4)nc(-c4ccccc4)n3)cc2-c2ccccc2-c2nc(-c3ccccc3)nc(-c3ccccc3-c3ccccc3)n2)c1 ; N#Cc1ccc(-c2ccc(-c3nc(-c4ccccc4)nc(-c4ccccc4)n3)cc2-c2ccccc2-c2nc(-c3ccccc3)nc(-c3cccc(-c4ccccc4)c3)n2)cc1]

The substituent of the compound of Chemical Formula 1 may be bonded by a method known in the art, and the type and position of the substituent or the number of substituents may be changed according to the technology known in the art.

In addition, various substituents may be introduced into the core structure having the structure described above to synthesize compounds having inherent characteristics of the introduced substituents. For example, a substituent usually used for a hole injection layer material, a material for transporting holes, a light emitting layer material, and an electron transport layer material, which are used for manufacturing an organic light emitting device, may be introduced into the core structure to synthesize a material which satisfies conditions required for each organic material layer.

Furthermore, the organic light emitting device according to the present invention is an organic light emitting device including: a first electrode; a second electrode provided to face the first electrode; and an organic material layer having one or more layers provided between the first electrode and the second electrode, in which one or more layers of the organic material layer include the above-described compound.

The organic light emitting device of the present invention may be manufactured using typical manufacturing methods and materials of an organic light emitting device, except that the above-described compound is used to form an organic material layer having one or more layers.

The compound may be formed as an organic material layer by not only a vacuum deposition method, but also a solution coating method when an organic light emitting device is manufactured. Herein, the solution coating method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a layer which injects and transports holes simultaneously, a light emitting layer, an electron transport layer, an electron injection layer, and the like as organic material layers. However, the structure of the organic light emitting device is not limited thereto, and may include a fewer or greater number of organic material layers.

In the organic light emitting device of the present invention, the organic material layer may include one or more layers of an electron transport layer, an electron injection layer, and an electron injection and transport layer, and one or more layers of the layers may include the compound of Chemical Formula 1.

In another organic light emitting device, the organic material layer may include an electron transport layer or an electron injection layer, and the electron transport layer or the electron injection layer may include the compound of Chemical Formula 1.

In the organic light emitting device of the present invention, the electron injection and transport layer includes the compound of Chemical Formula 1 and a metal complex.

In the organic light emitting device of the present invention, the organic material layer may include one or more layers of a hole injection layer, a hole transport layer, and a layer which injects and transports holes simultaneously, and one or more layers of the layers may include the compound of Chemical Formula 1.

In still another organic light emitting device, the organic material layer may include a hole injection layer or a hole transport layer, and the hole transport layer or the hole injection layer may include the compound of Chemical Formula 1.

In one embodiment of the present specification, the first electrode is a positive electrode, and the second electrode is a negative electrode.

According to another embodiment, the first electrode is a negative electrode, and the second electrode is a positive electrode.
(1) Positive electrode/Hole transport layer/Light emitting layer/Negative electrode
(2) Positive electrode/Hole injection layer/Hole transport layer/Light emitting layer/Negative electrode
(3) Positive electrode/Hole injection layer/Hole buffer layer/Hole transport layer/Light emitting layer/Negative electrode
(4) Positive electrode/Hole transport layer/Light emitting layer/Electron transport layer/Negative electrode
(5) Positive electrode/Hole transport layer/Light emitting layer/Electron transport layer/Electron injection layer/Negative electrode
(6) Positive electrode/Hole injection layer/Hole transport layer/Light emitting layer/Electron transport layer/Negative electrode
(7) Positive electrode/Hole injection layer/Hole transport layer/Light emitting layer/Electron transport layer/Electron injection layer/Negative electrode
(8) Positive electrode/Hole injection layer/Hole buffer layer/Hole transport layer/Light emitting layer/Electron transport layer/Negative electrode
(9) Positive electrode/Hole injection layer/Hole buffer layer/Hole transport layer/Light emitting layer/Electron transport layer/Electron injection layer/Negative electrode
(10) Positive electrode/Hole transport layer/Electron blocking layer/Light emitting layer/Electron transport layer/Negative electrode
(11) Positive electrode/Hole transport layer/Electron blocking layer/Light emitting layer/Electron transport layer/Electron injection layer/Negative electrode
(12) Positive electrode/Hole injection layer/Hole transport layer/Electron blocking layer/Light emitting layer/Electron transport layer/Negative electrode
(13) Positive electrode/Hole injection layer/Hole transport layer/Electron blocking layer/Light emitting layer/Electron transport layer/Electron injection layer/Negative electrode
(14) Positive electrode/Hole transport layer/Light emitting layer/Hole blocking layer/Electron transport layer/Negative electrode
(15) Positive electrode/Hole transport layer/Light emitting layer/Hole blocking layer/Electron transport layer/Electron injection layer/Negative electrode
(16) Positive electrode/Hole injection layer/Hole transport layer/Light emitting layer/Hole blocking layer/Electron transport layer/Negative electrode
(17) Positive electrode/Hole injection layer/Hole transport layer/Light emitting layer/Hole blocking layer/Electron transport layer/Electron injection layer/Negative electrode
(18) Positive electrode/Hole injection layer/Hole transport layer/Electron blocking layer/Light emitting layer/Hole blocking layer/Electron injection and transport layer/Negative electrode

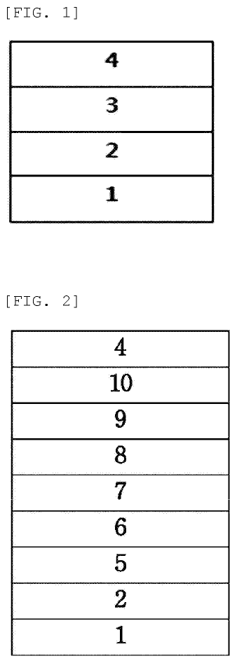

The structure of the organic light emitting device of the present invention may have a structure as illustrated in FIGS. 1 and 2, but is not limited thereto.

FIG. 1 exemplifies the structure of an organic light emitting device in which a positive electrode 2, an organic material layer 3, and a negative electrode 4 are sequentially stacked on a substrate 1. In the structure described above, the compound of Chemical Formula 1 may be included in the organic material layer 3.

FIG. 1 exemplifies the structure of an organic light emitting device in which a positive electrode 2, an organic material layer 3, and a negative electrode 4 are sequentially stacked on a substrate 1. In the structure described above, the compound of Chemical Formula 1 may be included in the organic material layer 3.

FIG. 2 exemplifies the structure of an organic light emitting device in which a positive electrode 2, a hole injection layer 5, a hole transport layer 6, an electron blocking layer 7, a light emitting layer 8, a hole blocking layer 9, an electron injection and transport layer 10, and a negative electrode 4 are sequentially stacked on a substrate 1. The compound of Chemical Formula 1 may be included in the hole blocking layer 9 or the electron injection and transport layer 10.

For example, the organic light emitting device according to the present invention may be manufactured by depositing a metal or a metal oxide having conductivity, or an alloy thereof on a substrate to form a positive electrode, forming an organic material layer having one or more layers selected from the group consisting of a hole injection layer, a hole transport layer, a layer which transports and injects holes simultaneously, a light emitting layer, an electron transport layer, an electron injection layer, and a layer which transports and injects electrons simultaneously, thereon, and then depositing a material, which may be used as a negative electrode, thereon, by using a physical vapor deposition (PVD) method such as sputtering or e-beam evaporation. In addition to the method described above, an organic light emitting device may also be made by sequentially depositing a negative electrode material, an organic material layer, and a positive electrode material on a substrate.

The organic material layer may have a multi-layered structure including a hole injection layer, a hole transport layer, a light emitting layer, and an electron transport layer, and the like, but is not limited thereto and may have a single-layered structure. Further, the organic material layer may be manufactured to include a fewer number of layers by a method such as a solvent process, for example, spin coating, dip coating, doctor blading, screen printing, inkjet printing, or a thermal transfer method, using various polymer materials, instead of a deposition method.

The positive electrode is an electrode which injects holes, and as a positive electrode material, materials having a high work function are usually preferred so as to facilitate the injection of holes into an organic material layer. Specific examples of the positive electrode material capable of being used in the present disclosure include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, but are not limited thereto.

The negative electrode is an electrode injecting electrons, and as the negative electrode material, materials having small work function are normally preferred so that electron injection to an organic material layer is smooth. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

The hole injection layer is a layer which serves to facilitate the injection of holes from a positive electrode to a light emitting layer, and a hole injection material is preferably a material which may proficiently accept holes from a positive electrode at a low voltage, and the highest occupied molecular orbital (HOMO) of the hole injection material is preferably a value between the work function of the positive electrode material and the HOMO of the neighboring organic material layer. Specific examples of the hole injection material include metal porphyrin, oligothiophene, arylamine-based organic materials, hexanitrile hexaazatriphenylene-based organic materials, quinacridone-based organic materials, perylene-based organic materials, anthraquinone, polyaniline-based and polythiophene-based conductive polymers, and the like, but are not limited thereto. The hole injection layer may have a thickness of 1 to 150 nm. When the hole injection layer has a thickness of 1 nm or more, there is an advantage in that it is possible to prevent hole injection characteristics from deteriorating, and when the hole injection layer has a thickness of 150 nm or less, there is an advantage in that it is possible to prevent the driving voltage from being increased in order to improve the movement of holes due to the too thick hole injection layer.

According to an exemplary embodiment of the present specification, the hole injection layer includes a compound of the following Chemical Formula HI-1, but is not limited thereto.

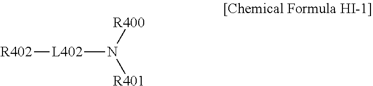

In Chemical Formula HI-1,
R400 to R402 are the same as or different from each other, and are each independently any one selected from the group consisting of hydrogen; deuterium; a substituted or unsubstituted alkyl group; a substituted or unsubstituted aryl group; a substituted or unsubstituted amine group; a substituted or unsubstituted heteroaryl group; and a combination thereof, or are bonded to an adjacent group to form a substituted or unsubstituted ring, and
L402 is a substituted or unsubstituted arylene group, or a substituted or unsubstituted heteroarylene group.

According to an exemplary embodiment of the present specification, R400 to R402 are the same as or different from each other, and are each independently any one selected from the group consisting of a substituted or unsubstituted aryl group; a substituted or unsubstituted amine group; a substituted or unsubstituted heteroaryl group; and a combination thereof.

According to an exemplary embodiment of the present specification, R402 is any one selected from the group consisting of a phenyl group substituted with a carbazole group or an arylamine group; a biphenyl group substituted with a carbazole group or an arylamine group; and a combination thereof.

According to an exemplary embodiment of the present specification, R400 and R401 are the same as or different from each other, and are each independently a substituted or unsubstituted aryl group, or are bonded to an adjacent group to form an aromatic hydrocarbon ring substituted with an alkyl group.

According to an exemplary embodiment of the present specification, R400 and R401 are the same as or different from each other, and are each independently an aryl group unsubstituted or substituted with an alkyl group.

According to an exemplary embodiment of the present specification, R400 and R401 are the same as or different from each other, and are each independently a phenyl group or a dimethylfluorene group.

According to an exemplary embodiment of the present specification, a compound of Chemical Formula HI-1 is the following compound.

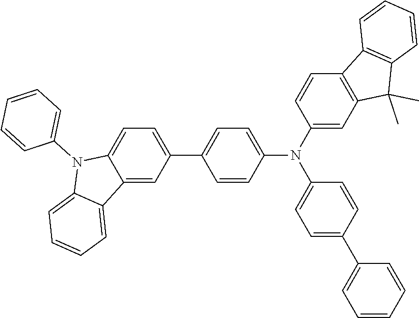 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

According to an exemplary embodiment of the present specification, the hole injection layer includes a compound of the following Chemical Formula HI-1, but is not limited thereto.

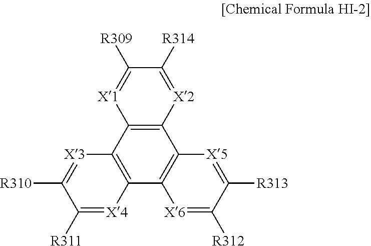

In Chemical Formula HI-2,
at least one of X′1 to X′6 is N, and the others are CH, and
R309 to R314 are the same as or different from each other, and are each independently hydrogen; deuterium; a nitrile group; a substituted or unsubstituted alkyl group; a substituted or unsubstituted amine group; a substituted or unsubstituted aryl group; or a substituted or unsubstituted heteroaryl group, or are bonded to an adjacent group to form a substituted or unsubstituted ring.

According to an exemplary embodiment of the present specification, X′1 to X′6 are N.

According to an exemplary embodiment of the present specification, R309 to R314 are a nitrile group.

According to an exemplary embodiment of the present specification, a compound of Chemical Formula HI-2 is the following compound.

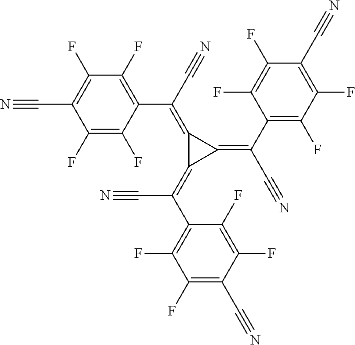 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

The hole transfer layer may perform a role of smoothly transferring holes. A hole transport material is suitably a material having high hole mobility which may accept holes from a positive electrode or a hole injection layer and transfer the holes to a light emitting layer. Specific examples thereof include arylamine-based organic materials, conductive polymers, block copolymers having both conjugated portions and non-conjugated portions, and the like, but are not limited thereto.

According to an exemplary embodiment of the present specification, the hole transport layer includes a compound of the following Chemical Formula HT-2, but is not limited thereto.

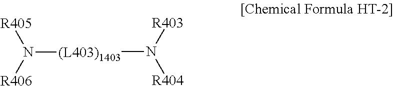

In Chemical Formula HT-2,
R403 to R406 are the same as or different from each other, and are each independently any one selected from the group consisting of hydrogen; deuterium; a substituted or unsubstituted alkyl group; a substituted or unsubstituted aryl group; a substituted or unsubstituted amine group; a substituted or unsubstituted heteroaryl group; and a combination thereof, or are bonded to an adjacent group to form a substituted or unsubstituted ring,
L403 is a substituted or unsubstituted arylene group, or a substituted or unsubstituted heteroarylene group, and
1403 is an integer from 1 to 3, and when 1403 is 2 or greater, L403's are the same as or different from each other.

According to an exemplary embodiment of the present specification, R403 to R406 are the same as or different from each other, and are each independently any one selected from the group consisting of a substituted or unsubstituted aryl group; a substituted or unsubstituted amine group; a substituted or unsubstituted heteroaryl group; and a combination thereof.

According to an exemplary embodiment of the present specification, R403 to R406 are the same as or different from each other, and are each independently an aryl group having 6 to 30 carbon atoms.

According to an exemplary embodiment of the present specification, R403 to R406 are the same as or different from each other, and are each independently a phenyl group, a biphenyl group, or a naphthyl group.

According to an exemplary embodiment of the present specification, R403 to R406 are the same as or different from each other, and are each independently a phenyl group.

According to an exemplary embodiment of the present specification, L403 is an arylene group having 6 to 30 carbon atoms, or a heteroarylene group having 3 to 30 carbon atoms, which is substituted with an aryl group.

According to an exemplary embodiment of the present specification, L403 is a phenylene group, a divalent biphenyl group, or a divalent carbazole group unsubstituted or substituted with an aryl group.

According to an exemplary embodiment of the present specification, a compound of Chemical Formula HT-2 is the following compound.

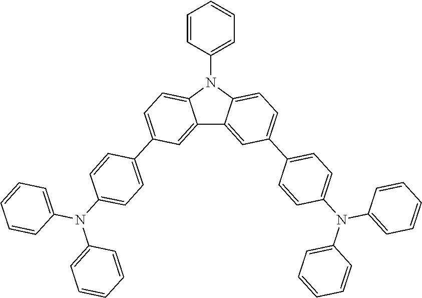 [SMILES: c1ccc(N(c2ccccc2)c2ccc(-c3ccc4c(c3)c3cc(-c5ccc(N(c6ccccc6)c6ccccc6)cc5)ccc3n4-c3ccccc3)cc2)cc1]

An electron blocking layer may be provided between a hole transport layer and a light emitting layer. As the electron blocking layer, the above-described spiro compound or a material known in the art may be used.

According to an exemplary embodiment of the present specification, the electron blocking layer includes a compound of the following Chemical Formula EB-1, but is not limited thereto.

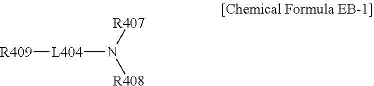

In Chemical Formula EB-1,
R407 to R409 are the same as or different from each other, and are each independently any one selected from the group consisting of hydrogen; deuterium; a substituted or unsubstituted alkyl group; a substituted or unsubstituted aryl group; a substituted or unsubstituted amine group; a substituted or unsubstituted heteroaryl group; and a combination thereof, or are bonded to an adjacent group to form a substituted or unsubstituted ring, and
L404 is a substituted or unsubstituted arylene group, or a substituted or unsubstituted heteroarylene group.
L404 is a phenylene group.

According to an exemplary embodiment of the present specification, R407 to R409 are the same as or different from each other, and are each independently any one selected from the group consisting of a substituted or unsubstituted aryl group; a substituted or unsubstituted amine group; a substituted or unsubstituted heteroaryl group; and a combination thereof.

According to an exemplary embodiment of the present specification, R409 is a carbazole group unsubstituted or substituted with a phenyl group.

According to an exemplary embodiment of the present specification, R407 and R408 are the same as or different from each other, and are each independently an aryl group unsubstituted or substituted with an alkyl group, or are bonded to an adjacent group to form an aromatic hydrocarbon ring substituted with an alkyl group.

According to an exemplary embodiment of the present specification, R407 and R408 are the same as or different from each other, and are each independently a phenyl group, a biphenyl group, or a dimethylfluorene group.

According to an exemplary embodiment of the present specification, a compound of Chemical Formula EB-1 is the following compound.

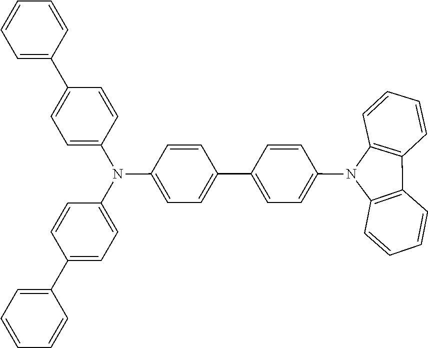 [SMILES: c1ccc(-c2ccc(N(c3ccc(-c4ccccc4)cc3)c3ccc(-c4ccc(-n5c6ccccc6c6ccccc65)cc4)cc3)cc2)cc1]

The light emitting layer may emit red, green, or blue light, and may be composed of a phosphorescent material or a fluorescent material. The light emitting material is a material which may receive holes and electrons from a hole transport layer and an electron transport layer, respectively, and combine the holes and the electrons to emit light in a visible ray region, and is preferably a material having high quantum efficiency for fluorescence or phosphorescence. Specific examples thereof include: 8-hydroxy-quinoline aluminum complexes (Alq₃); carbazole-based compounds; dimerized styryl compounds; BAlq; 10-hydroxybenzoquinoline-metal compounds; benzoxazole-based, benzothiazole-based and benzimidazole-based compounds; poly(p-phenylenevinylene) (PPV)-based polymers; spiro compounds; polyfluorene, rubrene, and the like, but are not limited thereto.

Examples of the host material for the light emitting layer include condensed aromatic ring derivatives, or hetero ring-containing compounds, and the like. Specifically, examples of the condensed aromatic ring derivative include anthracene derivatives, pyrene derivatives, naphthalene derivatives, pentacene derivatives, phenanthrene compounds, fluoranthene compounds, and the like, and examples of the hetero ring-containing compound include carbazole derivatives, dibenzofuran derivatives, ladder-type furan compounds, pyrimidine derivatives, and the like, but the examples thereof are not limited thereto.

According to an exemplary embodiment of the present specification, the host includes a compound of the following Chemical Formula H-1, but is not limited thereto.

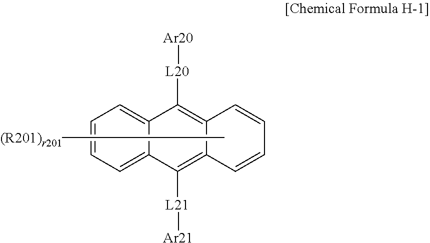

In Chemical Formula H-1,
L20 and L21 are the same as or different from each other, and are each independently a direct bond; a substituted or unsubstituted arylene group; or a substituted or unsubstituted divalent heterocyclic group,
Ar20 and Ar21 are the same as or different from each other, and are each independently hydrogen; deuterium; a substituted or unsubstituted aryl group; or a substituted or unsubstituted heterocyclic group,
R201 is hydrogen; deuterium; a halogen group; a substituted or unsubstituted alkyl group; a substituted or unsubstituted cycloalkyl group; a substituted or unsubstituted aryl group; or a substituted or unsubstituted heterocyclic group, and
r201 is an integer from 1 to 8, and when r201 is 2 or greater, two or more R201's are the same as or different from each other.

In an exemplary embodiment of the present specification, L20 and L21 are the same as or different from each other, and are each independently a direct bond; a monocyclic or polycyclic arylene group having 6 to 30 carbon atoms; or a monocyclic or polycyclic divalent heterocyclic group having 2 to 30 carbon atoms.

In an exemplary embodiment of the present specification, L20 and L21 are the same as or different from each other, and are each independently a direct bond; a phenylene group which is unsubstituted or substituted with deuterium; a biphenylylene group which is unsubstituted or substituted with deuterium; a naphthylene group which is unsubstituted or substituted with deuterium; a divalent dibenzofuran group; or a divalent dibenzothiophene group.

In an exemplary embodiment of the present specification, Ar20 is a substituted or unsubstituted heterocyclic group, and Ar21 is a substituted or unsubstituted aryl group.

In an exemplary embodiment of the present specification, Ar20 and Ar21 are the same as or different from each other, and are each independently a substituted or unsubstituted monocyclic or polycyclic aryl group having 6 to 30 carbon atoms; or a substituted or unsubstituted monocyclic or polycyclic heterocyclic group having 2 to 30 carbon atoms.

In an exemplary embodiment of the present specification, Ar20 and Ar21 are the same as or different from each other, and are each independently a substituted or unsubstituted monocyclic to tetracyclic aryl group having 6 to 20 carbon atoms; or a substituted or unsubstituted monocyclic to tetracyclic heterocyclic group having 6 to 20 carbon atoms.

In an exemplary embodiment of the present specification, Ar20 and Ar21 are the same as or different from each other, and are each independently a phenyl group which is unsubstituted or substituted with deuterium or a monocyclic or polycyclic aryl group having 6 to 20 carbon atoms; a biphenyl group which is unsubstituted or substituted with deuterium or a monocyclic or polycyclic aryl group having 6 to 20 carbon atoms; a naphthyl group which is unsubstituted or substituted with a monocyclic or polycyclic aryl group having 6 to 20 carbon atoms; a thiophene group which is unsubstituted or substituted with a monocyclic or polycyclic aryl group having 6 to 30 carbon atoms; a dibenzofuran group which is unsubstituted or substituted with a monocyclic or polycyclic aryl group having 6 to 20 carbon atoms; a naphthobenzofuran group which is unsubstituted or substituted with a monocyclic or polycyclic aryl group having 6 to 20 carbon atoms; a dibenzothiophene group which is unsubstituted or substituted with a monocyclic or polycyclic aryl group having 6 to 20 carbon atoms; or a naphthobenzothiophene group which is unsubstituted or substituted with a monocyclic or polycyclic aryl group having 6 to 20 carbon atoms.

In an exemplary embodiment of the present specification, Ar20 and Ar21 are the same as or different from each other, and are each independently a phenyl group which is unsubstituted or substituted with deuterium; a biphenyl group which is unsubstituted or substituted with deuterium; a terphenyl group; a naphthyl group which is unsubstituted or substituted with deuterium; a thiophene group which is unsubstituted or substituted with a phenyl group; a phenanthrene group; a dibenzofuran group; a naphthobenzofuran group; a dibenzothiophene group; or a naphthobenzothiophene group.

In an exemplary embodiment of the present specification, Ar20 and Ar21 are the same as or different from each other, and are each independently a 1-naphthyl group, or a 2-naphthyl group.

According to an exemplary embodiment of the present specification, R201 is hydrogen.

According to an exemplary embodiment of the present specification, a compound of Chemical Formula H-1 is the following compound.

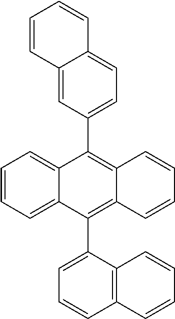 [SMILES: c1ccc2cc(-c3c4ccccc4c(-c4cccc5ccccc45)c4ccccc34)ccc2c1]

When the light emitting layer emits red light, phosphorescent materials such as bis(1-phenylisoquinoline)acetylacetonate iridium (PIQIr(acac)), bis(1-phenylquinoline)acetylacetonate iridium (PQIr(acac)), tris(1-phenylquinoline)iridium (PQIr) or octaethylporphyrin platinum (PtOEP), or fluorescent materials such as tris(8-hydroxyquinolino)aluminum (Alq₃) may be used as the light emitting dopant, however, the light emitting dopant is not limited thereto. When the light emitting layer emits green light, it is possible to use a phosphorescent material such as fac tris(2-phenylpyridine)iridium (Ir(ppy)₃), or a fluorescent material such as tris(8-hydroxyquinolino)aluminum (Alq₃), as the light emitting dopant, but the light emitting dopant is not limited thereto. When the light emitting layer emits blue light, phosphorescent materials such as (4,6-F2ppy)₂Irpic, or fluorescent materials such as spiro-DPVBi, spiro-6P, polymers or PPV-based polymers may be used as the light emitting dopant, however, the light emitting dopant is not limited thereto.

According to an exemplary embodiment of the present specification, the dopant is a compound of the following Chemical Formula D-1.

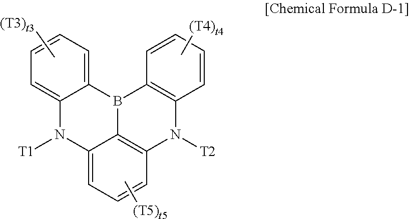

In Chemical Formula D-1,
T1 to T5 are the same as or different from each other, and are each independently hydrogen; a substituted or unsubstituted alkyl group; a substituted or unsubstituted amine group; or a substituted or unsubstituted aryl group, t3 and t4 are each an integer from 1 to 4,
t5 is an integer from 1 to 3,
when t3 is 2 or greater, two or more T3's are the same as or different from each other,
when t4 is 2 or greater, two or more T4's are the same as or different from each other, and
when t5 is 2 or greater, two or more T5's are the same as or different from each other.

According to an exemplary embodiment of the present specification, T1 to T5 are the same as or different from each other, and are each independently hydrogen; a substituted or unsubstituted straight-chained or branched alkyl group having 1 to 30 carbon atoms; a substituted or unsubstituted monocyclic or polycyclic arylamine group having 6 to 30 carbon atoms; or a substituted or unsubstituted monocyclic or polycyclic aryl group having 6 to 30 carbon atoms.

According to an exemplary embodiment of the present specification, T1 to T5 are the same as or different from each other, and are each independently hydrogen; a straight-chained or branched alkyl group having 1 to 30 carbon atoms; a monocyclic or polycyclic arylamine group having 6 to 30 carbon atoms; or a monocyclic or polycyclic aryl group having 6 to 30 carbon atoms, which is unsubstituted or substituted with a straight-chained or branched alkyl group having 1 to 30 carbon atoms.

According to an exemplary embodiment of the present specification, T1 to T5 are the same as or different from each other, and are each independently hydrogen; a methyl group; an isopropyl group; a diphenylamine group; or a phenyl group that is unsubstituted or substituted with a methyl group or an isopropyl group.

According to an exemplary embodiment of the present specification, T1 to T5 are the same as or different from each other, and are each independently an isopropyl group; or a phenyl group unsubstituted or substituted with an isopropyl group.

According to an exemplary embodiment of the present specification, a compound of Chemical Formula D-1 is the following compound.

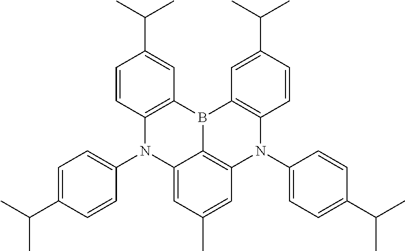 [SMILES: Cc1cc2c3c(c1)N(c1ccc(C(C)C)cc1)c1ccc(C(C)C)cc1B3c1cc(C(C)C)ccc1N2c1ccc(C(C)C)cc1]

A hole blocking layer may be provided between the electron transport layer and the light emitting layer, and materials known in the art may be used.

According to an exemplary embodiment of the present specification, the hole blocking layer includes a compound of the following Chemical Formula HB-1.

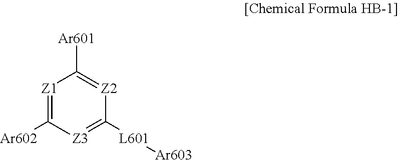

In Chemical Formula HB-1,
at least one of Z1 to Z3 is N, and the others are CH,
L601 is a direct bond; a substituted or unsubstituted arylene group; or a substituted or unsubstituted heteroarylene group, and
Ar601 to Ar603 are the same as or different from each other, and are each independently a substituted or unsubstituted aryl group; or a substituted or unsubstituted heteroaryl group.

According to an exemplary embodiment of the present specification, L601 is a substituted or unsubstituted monocyclic or polycyclic arylene group having 6 to 30 carbon atoms.

According to an exemplary embodiment of the present specification, L601 is a phenylene group; a biphenylylene group; or a naphthylene group.

According to an exemplary embodiment of the present specification, Ar601 to Ar603 are the same as or different from each other, and are each independently a substituted or unsubstituted monocyclic or polycyclic aryl group having 6 to 30 carbon atoms, or a heteroaryl group having 3 to 30 carbon atoms.

According to an exemplary embodiment of the present specification, Ar601 to Ar603 is a phenyl group, or a dimethylfluorene group.

According to an exemplary embodiment of the present specification, a compound of Chemical Formula HB-1 is the following compound.

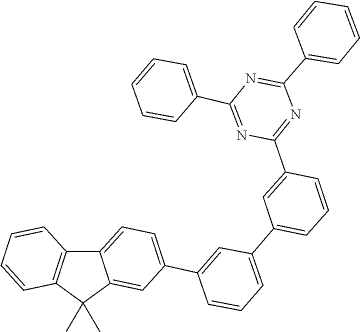 [SMILES: CC1(C)c2ccccc2-c2ccc(-c3cccc(-c4cccc(-c5nc(-c6ccccc6)nc(-c6ccccc6)n5)c4)c3)cc21]

The electron transfer layer may perform a role of smoothly transferring electrons. As the electron transfer material, materials capable of favorably receiving electrons from a negative electrode, moving the electrons to a light emitting layer, and having high mobility for the electrons are suited. Specific examples thereof include Al complexes of 8-hydroxyquinoline; complexes including Alq₃; organic radical compounds; hydroxyflavone-metal complexes, and the like, but are not limited thereto. The electron transport layer may have a thickness of 1 to 50 nm. When the electron transport layer has a thickness of 1 nm or more, there is an advantage in that it is possible to prevent electron transport characteristics from deteriorating, and when the electron transport layer has a thickness of 50 nm or less, there is an advantage in that it is possible to prevent the driving voltage from being increased in order to improve the movement of electrons due to the too thick electron transport layer.

The electron injection layer may perform a role of smoothly injecting electrons. An electron injection material is preferably a compound which has a capability of transporting electrons, an effect of injecting electrons from a negative electrode, and an excellent effect of injecting electrons into a light emitting layer or a light emitting material, prevents excitons produced from a light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylene tetracarboxylic acid, fluorenylidene methane, anthrone or the like, and derivatives thereof, metal complex compounds, nitrogen-containing 5-membered ring derivatives, and the like, but are not limited thereto.

The electron injection and transport layer may be manufactured by appropriately selecting materials used for the electron injection layer and the electron transport layer.

The electron injection and transport layer may be manufactured using the compound of Chemical Formula 1 and a metal complex together.

The electron injection and transport layer includes the compound of Chemical Formula 1 and the metal complex at a weight ratio of 1:10 to 10:1.

The electron injection and transport layer includes the compound of Chemical Formula 1 and the metal complex at a weight ratio of 1:3 to 3:1.

The metal complex compound includes 8-hydroxyquinolinato lithium, bis(8-hydroxyquinolinato)zinc, bis(8-hydroxyquinolinato)copper, bis(8-hydroxyquinolinato)-manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato) (o-cresolato)gallium, bis(2-methyl-8-quinolinato) (1-naphtholato)aluminum, bis(2-methyl-8-quinolinato) (2-naphtholato)gallium and the like, but is not limited thereto.

The hole blocking layer is a layer which blocks holes from reaching a negative electrode, and may be generally formed under the same conditions as those of the hole injection layer. Specific examples thereof include oxadiazole derivatives or triazole derivatives, phenanthroline derivatives, BCP, aluminum complexes, and the like, but are not limited thereto.

The organic light emitting device according to the present invention may be a top emission type, a bottom emission type, or a dual emission type according to the material to be used.

### EXAMPLES

The organic light emitting device of the present invention may be manufactured using typical manufacturing methods and materials of an organic light emitting device, except that the above-described compound is used to form an organic material layer having one or more layers.

The preparation method of the compound of Chemical Formula 1 and the manufacture of an organic light emitting device using the same will be specifically described in the following Examples. However, the following Examples are provided for exemplifying the present invention, and the scope of the present invention is not limited thereby.

In the following reaction formulae, with respect to the type and number of substituent, various types of intermediates may be synthesized as a person skilled in the art appropriately selects a publicly-known starting material. As the type of reaction and the reaction condition, those known in the art may be used.

The preparation method of the compound of Chemical Formula 1 and the manufacture of an organic light emitting device using the same will be specifically described in the following Examples. However, the following Examples are provided for exemplifying the present invention, and the scope of the present invention is not limited thereby.

In the following reaction formulae, with respect to the type and number of substituent, various types of intermediates may be synthesized as a person skilled in the art appropriately selects a publicly-known starting material. As the type of reaction and the reaction condition, those known in the art may be used.

### Preparation Example 1. Synthesis of Compound E1

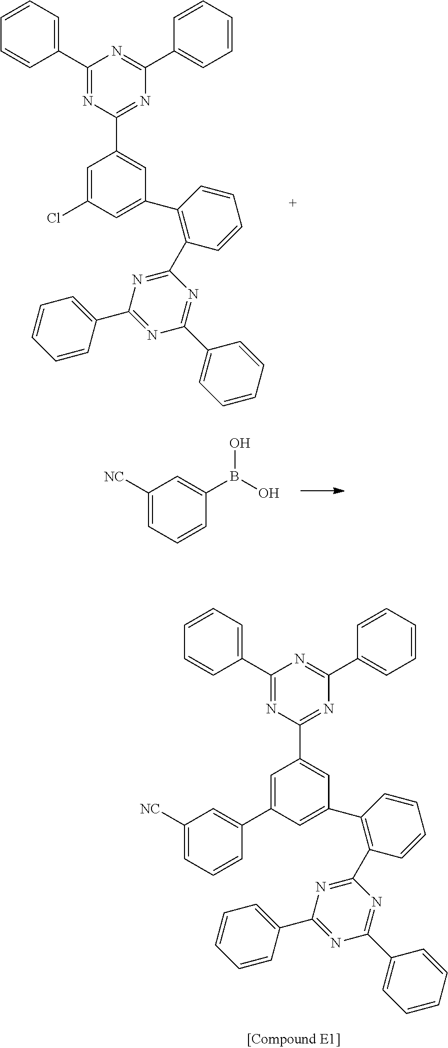

After the compound 6,6′-(5′-chloro-[1,1′-biphenyl]-2,3′-diyl)bis(2,4-diphenyl-1,3,5-triazine) (10.0 g, 15.3 mmol) and (3-cyanophenyl) boronic acid (2.25 g, 15.3 mmol) were completely dissolved in tetrahydrofuran (100 ml), a solution prepared by dissolving potassium carbonate (6.36 g, 46 mmol) in 20 ml of water was added thereto, and a solution prepared by dissolving tetrakistriphenylphosphinepalladium (354 mg, 0.3 mmol) in tetrahydrofuran was slowly added thereto. The temperature was lowered to room temperature, the reaction was terminated, and then the potassium carbonate solution was removed by filtering to recover a filtered white solid. The filtered white solid was washed twice with water and twice with ethyl acetate to prepare Compound E1 (9.7 g, yield 88%).

MS[M+H]⁺=717

### Preparation Example 2. Synthesis of Compound E2

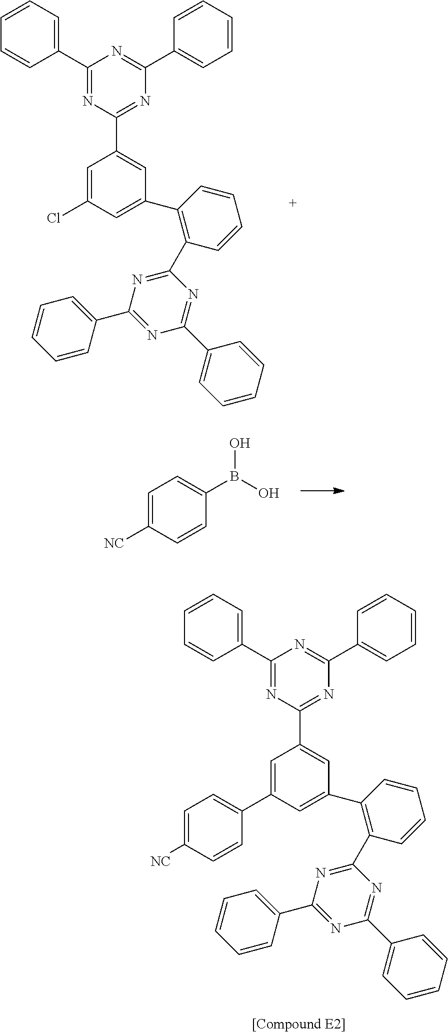

Compound E2 was prepared in the same manner as in Preparation Example 1, except that (4-cyanophenyl) boronic acid was used instead of the (3-cyanophenyl) boronic acid in Preparation Example 1.

MS[M+H]⁺=717

### Preparation Example 7. Synthesis of Compound E7

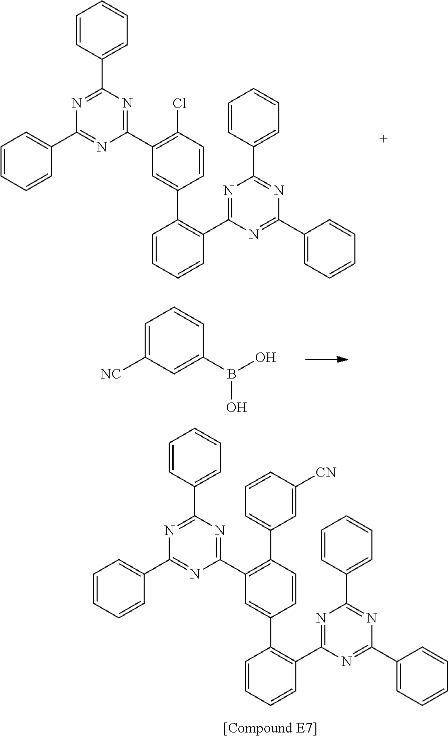

Compound E7 was prepared in the same manner as in Preparation Example 1, except that 6,6′-(4′-chloro-[1,1′-biphenylyl]-2,3′-diyl)bis(2,4-diphenyl-1,3,5-triazine) was used instead of 6,6′-(5′-chloro-[1,1′-biphenyl]-2,3′-diyl)bis(2,4-diphenyl-1,3,5-triazine) in Preparation Example 1.

MS[M+H]⁺=717

### Preparation Example 8. Synthesis of Compound E8

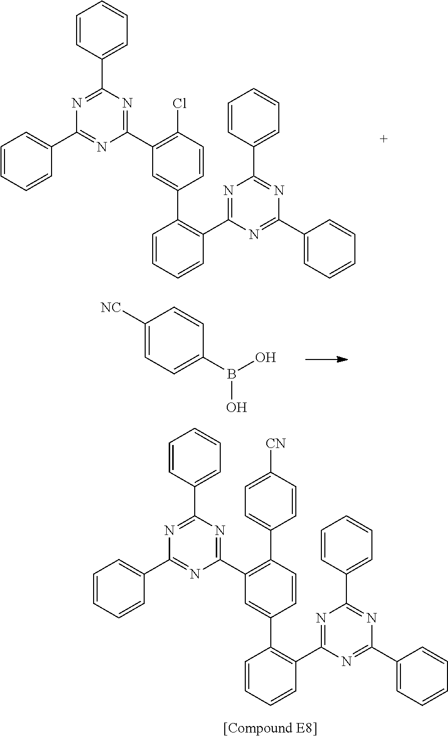

Compound E8 was prepared in the same manner as in Preparation Example 7, except that (4-cyanophenyl) boronic acid was used instead of the compound (3-cyanophenyl) boronic acid in Preparation Example 7.

MS[M+H]⁺=717

### Preparation Example 9. Synthesis of Compound E9

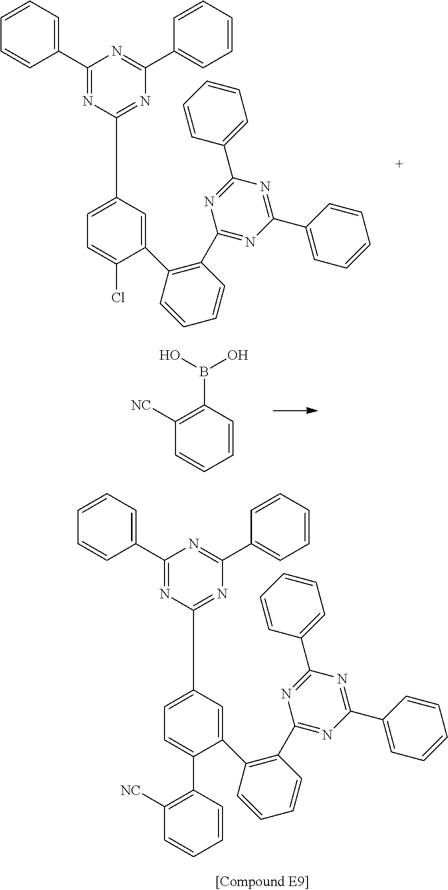

Compound E9 was prepared in the same manner as in Preparation Example 1, except that the compound 6,6′-(6′-chloro-[1,1′-biphenyl]-2,3′-diyl)bis(2,4-diphenyl-1,3,5-triazine) and (2-cyanophenyl) boronic acid were used instead of the compound 6,6′-(5′-chloro-[1,1′-biphenyl]-2,3′-diyl)bis(2,4-diphenyl-1,3,5-triazine) and (3-cyanophenyl) boronic acid, respectively, in Preparation Example 1.

MS[M+H]⁺=717

### Preparation Example 10. Synthesis of Compound E10

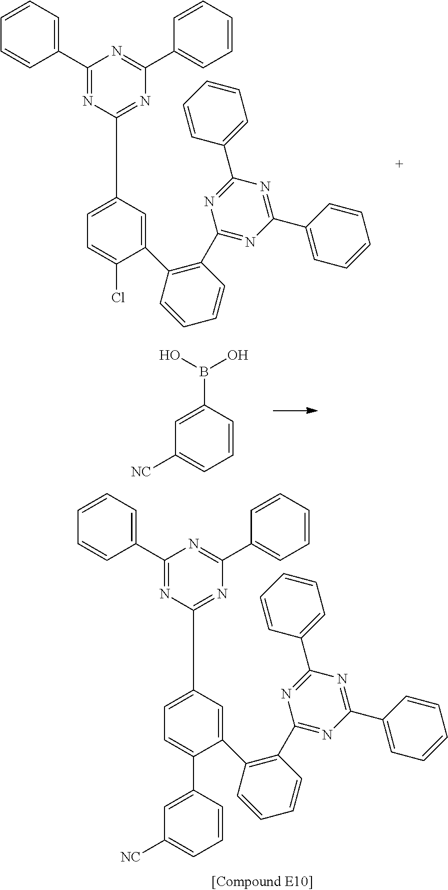

Compound E10 was prepared in the same manner as in Preparation Example 9, except that (3-cyanophenyl) boronic acid was used instead of the compound (2-cyanophenyl) boronic acid in Preparation Example 9.

MS[M+H]⁺=717

### Preparation Example 11. Synthesis of Compound E11

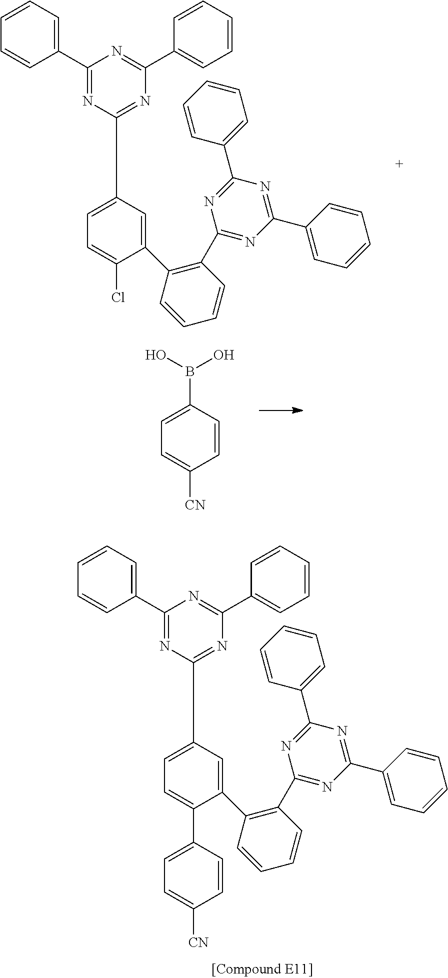

Compound E11 was prepared in the same manner as in Preparation Example 9, except that (4-cyanophenyl) boronic acid was used instead of the compound (2-cyanophenyl) boronic acid in Preparation Example 9.

MS[M+H]⁺=717

### Preparation Example 14. Synthesis of Compound E14

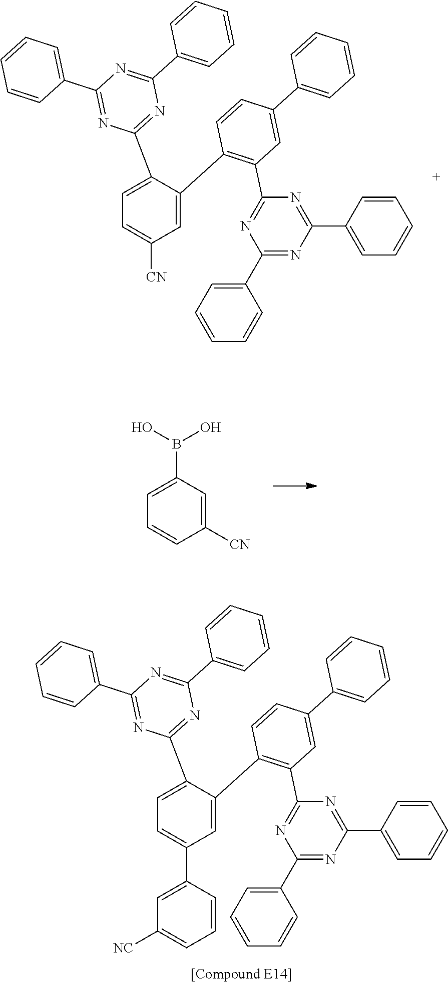 [SMILES: N#Cc1ccc(-c2nc(-c3ccccc3)nc(-c3ccccc3)n2)c(-c2ccc(-c3ccccc3)cc2-c2nc(-c3ccccc3)nc(-c3ccccc3)n2)c1 ; N#Cc1cccc(B(O)O)c1 ; N#Cc1cccc(-c2ccc(-c3nc(-c4ccccc4)nc(-c4ccccc4)n3)c(-c3ccc(-c4ccccc4)cc3-c3nc(-c4ccccc4)nc(-c4ccccc4)n3)c2)c1]

Compound E14 was prepared in the same manner as in Preparation Example 1, except that 2′,6-bis(4,6-diphenyl-1,3,5-triazin-2-yl)-[1,1′:4′,1″-terphenyl]-3-carbonitrile was used instead of the compound 6,6′-(5′-chloro-[1,1′-biphenyl]-2,3′-diyl)bis(2,4-diphenyl-1,3,5-triazine) in Preparation Example 1.

MS[M+H]⁺=793

### Preparation Example 15. Synthesis of Compound E15

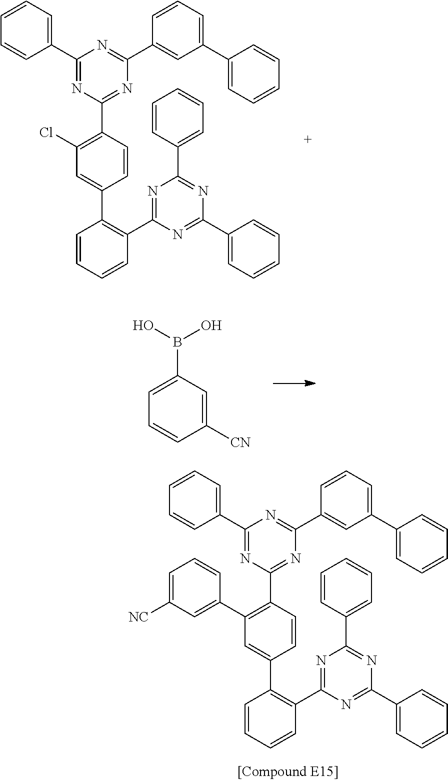

Compound E15 was prepared in the same manner as in Preparation Example 1, except that 2-([1,1′-biphenyl]-3-yl)-4-(3-chloro-2′-(4,6-diphenyl-1,3,5-triazin-2-yl)-[1,1′-biphenyl]-4-yl)-6-phenyl-1,3,5-triazine was used instead of the compound 6,6′-(5′-chloro-[1,1′-biphenyl]-2,3′-diyl)bis(2,4-diphenyl-1,3,5-triazine) in Preparation Example 1.

MS[M+H]⁺=793

### Preparation Example 16. Synthesis of Compound E16

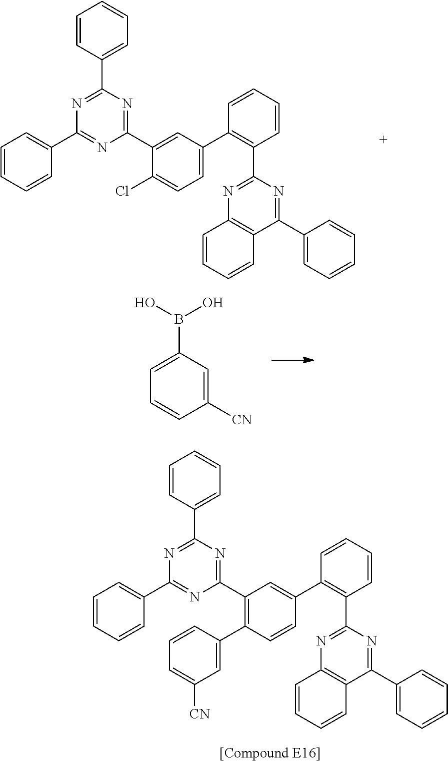

Compound E16 was prepared in the same manner as in Preparation Example 1, except that 2-(4′-chloro-3′-(4,6-diphenyl-1,3,5-triazin-2-yl)-[1,1′-biphenyl]-2-yl)-4-phenylquinazoline was used instead of the compound 6,6′-(5′-chloro-[1,1′-biphenyl]-2,3′-diyl)bis(2,4-diphenyl-1,3,5-triazine) in Preparation Example 1.

MS[M+H]⁺=690

When the preparation formula described in the Examples of the present specification and the intermediates are appropriately combined based on a typical technology common sense, all of the compounds of Chemical Formula 1 described in the present specification can be prepared.

### Example 1-1

A glass substrate thinly coated with indium tin oxide (ITO) to have a thickness of 1,000 Å was put into distilled water in which a detergent was dissolved, and ultrasonically washed. In this case, a product manufactured by the Fischer Co. was used as the detergent, and distilled water twice filtered using a filter manufactured by Millipore Co. was used as the distilled water. After the ITO was washed for 30 minutes, ultrasonic washing was repeated twice by using distilled water for 10 minutes. After the washing using distilled water was completed, ultrasonic washing was conducted by using isopropyl alcohol, acetone, and methanol solvents, and the resulting product was dried and then transported to a plasma washing machine. Furthermore, the substrate was cleaned by using oxygen plasma for 5 minutes, and then was transported to a vacuum deposition machine.

Compounds of the following Compound HI1 and the following Compound HI2 were thermally vacuum deposited to have a thickness of 100 Å at a ratio of 98:2 (molar ratio) on the transparent ITO electrode, which is the positive electrode thus prepared, thereby forming a hole injection layer. A compound of the following Chemical Formula HT1 (1,150 Å) was vacuum deposited on the hole injection layer, thereby forming a hole transport layer. Subsequently, a compound of the following EB1 was vacuum deposited to have a film thickness of 50 Å on the hole transport layer, thereby forming an electron blocking layer. Subsequently, a compound of the following Chemical Formula BH and a compound of the following Chemical Formula BD were vacuum deposited at a weight ratio of 50:1 to have a film thickness of 200 Å on the electron blocking layer, thereby forming a light emitting layer. A compound of the following Chemical Formula HB1 was vacuum deposited to have a film thickness of 50 Å on the light emitting layer, thereby forming a hole blocking layer. Subsequently, a compound of the following Chemical Formula E-1 and a compound of the following Chemical Formula LiQ were vacuum deposited at a weight ratio of 1:1 on the hole blocking layer, thereby forming an electron injection and transport layer having a thickness of 30 Å. Lithium fluoride (LiF) and aluminum were sequentially deposited on the electron injection and transport layer to have a thickness of 12 Å and 1,000 Å, respectively, thereby forming a negative electrode.

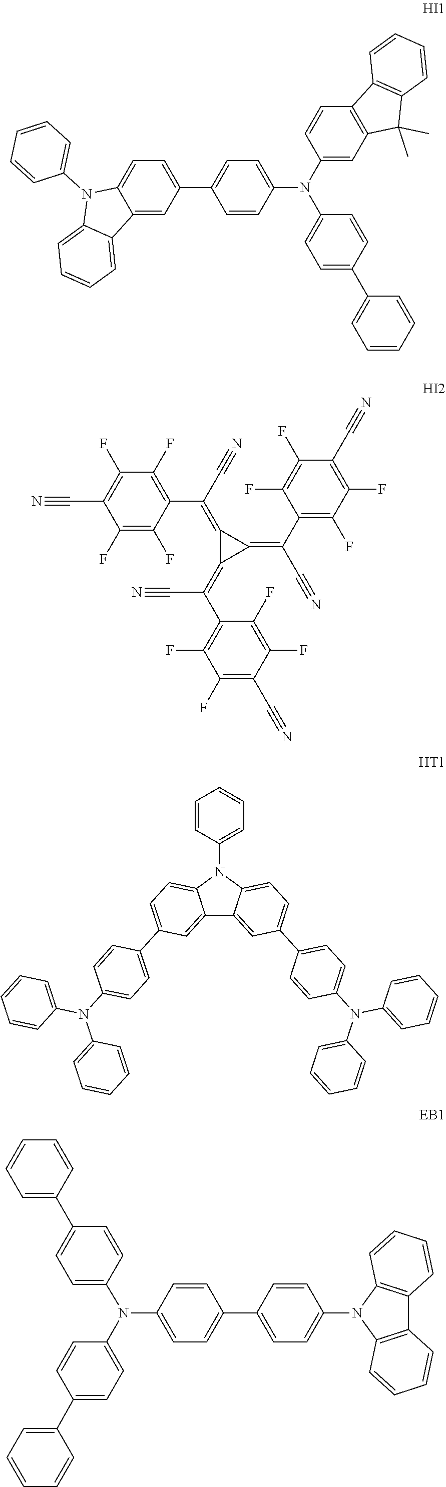 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(N(c2ccccc2)c2ccc(-c3ccc4c(c3)c3cc(-c5ccc(N(c6ccccc6)c6ccccc6)cc5)ccc3n4-c3ccccc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

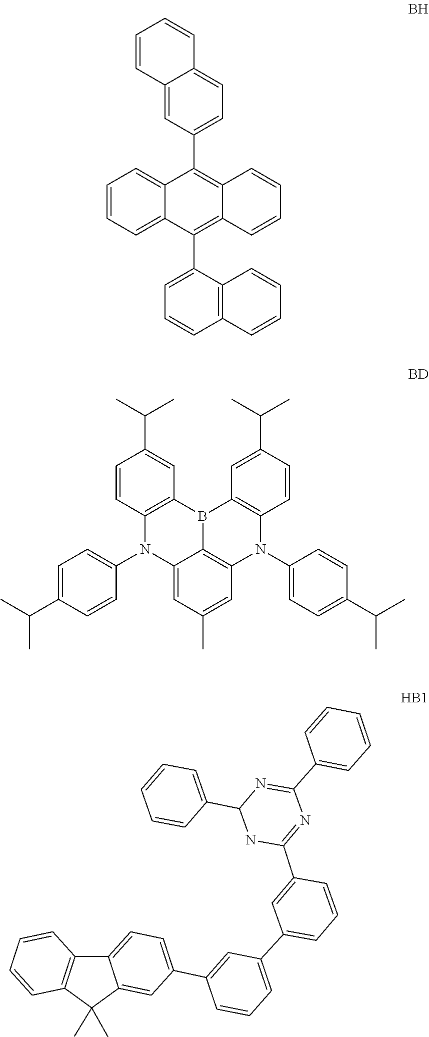

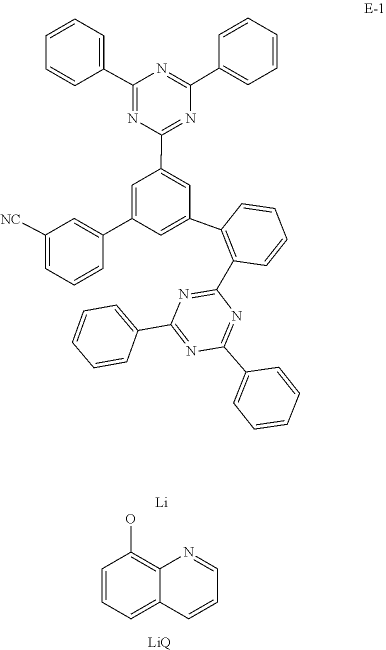

In the aforementioned procedure, the deposition rate of the organic material was maintained at 0.4 to 0.7 Å/sec, the deposition rates of lithium fluoride and aluminum of the negative electrode were maintained at 0.3 Å/sec and at 2 Å/sec, respectively, and the degree of vacuum during the deposition was maintained at 2×10⁻⁷ to 5×10⁻⁶ torr, thereby manufacturing an organic light emitting device.

### Examples 1-2 to 1-16

Organic light emitting devices were manufactured in the same manner as in Example 1-1, except that the compounds described in the following Table 1 were used instead of Compound E-1 in Example 1-1.

### Comparative Examples 1-1 to 1-6

Organic light emitting devices were manufactured in the same manner as in Example 1-1, except that the compounds described in the following Table 1 were used instead of Compound E-1. The compounds ET-1 to ET-6 used in the following Table 1 were as follows.

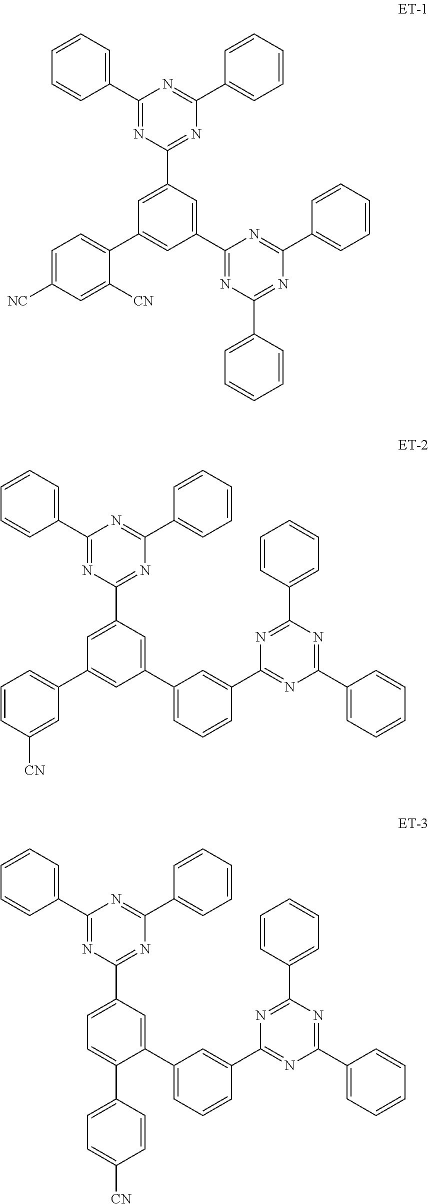 [SMILES: N#Cc1ccc(-c2cc(-c3nc(-c4ccccc4)nc(-c4ccccc4)n3)cc(-c3nc(-c4ccccc4)nc(-c4ccccc4)n3)c2)c(C#N)c1 ; N#Cc1cccc(-c2cc(-c3cccc(-c4nc(-c5ccccc5)nc(-c5ccccc5)n4)c3)cc(-c3nc(-c4ccccc4)nc(-c4ccccc4)n3)c2)c1 ; N#Cc1ccc(-c2ccc(-c3nc(-c4ccccc4)nc(-c4ccccc4)n3)cc2-c2cccc(-c3nc(-c4ccccc4)nc(-c4ccccc4)n3)c2)cc1]

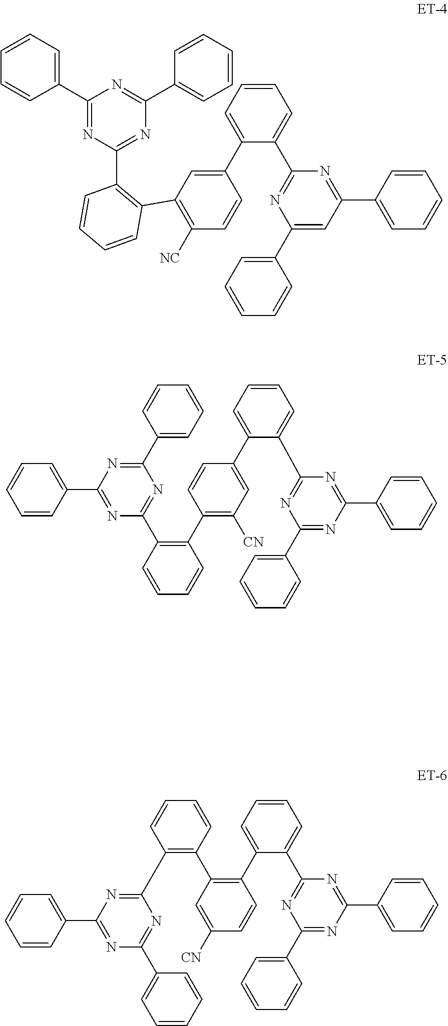

When current was applied to the organic light emitting devices manufactured in Examples 1-1 to 1-16 and Comparative Examples 1-1 to 1-6, the voltages, efficiencies, color coordinates, and service lives were measured, and the results are shown in the following [Table 1]. T95 means the time taken for the luminance to be reduced to 95% of the initial luminance (1600 nit).

TABLE 1

| | Compound | | | | |
|---|---|---|---|---|---|
| | (Electron | | | | |
| | injection and | voltage | efficiency | Color | |
| | transport | (V@20 | (cd/A@20 | coordinate | T95 |
| | layer) | mA/cm²) | mA/cm²) | (x, y) | (hr) |
| Example 1-1 | Compound | 4.32 | 5.92 | (0.138, | 238 |
| | E1 | | | 0.039) | |
| Example 1-2 | Compound | 4.35 | 6.12 | (0.138, | 238 |
| | E2 | | | 0.039) | |
| Example 1-7 | Compound | 4.38 | 6.45 | (0.137, | 241 |
| | E7 | | | 0.038) | |
| Example 1-8 | Compound | 4.31 | 6.30 | (0.138, | 254 |
| | E8 | | | 0.039) | |
| Example 1-9 | Compound | 4.29 | 6.29 | (0.139, | 249 |
| | E9 | | | 0.040) | |
| Example 1-10 | Compound | 4.36 | 6.31 | (0.140, | 251 |
| | E10 | | | 0.040) | |
| Example 1-11 | Compound | 4.36 | 6.17 | (0.138, | 238 |
| | E11 | | | 0.038) | |
| Example 1-14 | Compound | 4.24 | 6.10 | (0.139, | 246 |
| | E14 | | | 0.039) | |
| Example 1-15 | Compound | 4.42 | 6.21 | (0.138, | 238 |
| | E15 | | | 0.039) | |
| Example 1-16 | Compound | 4.43 | 6.37 | (0.139, | 230 |
| | E16 | | | 0.040) | |
| Comparative | ET-1 | 4.64 | 5.21 | (0.139, | 112 |
| Example 1-1 | | | | 0.040) | |
| Comparative | ET-2 | 4.59 | 4.98 | (0.138, | 131 |
| Example 1-2 | | | | 0.039) | |
| Comparative | ET-3 | 4.84 | 4.34 | (0.140, | 175 |
| Example 1-3 | | | | 0.038) | |
| Comparative | ET-4 | 4.76 | 4.56 | (0.140, | 168 |
| Example 1-4 | | | | 0.040) | |
| Comparative | ET-5 | 4.88 | 4.49 | (0.139, | 191 |
| Example 1-5 | | | | 0.040) | |
| Comparative | ET-6 | 4.83 | 4.51 | (0.140, | 188 |
| Example 1-6 | | | | 0.040) | |

As seen in Table 1, the organic light emitting device manufactured using the compound of the present invention as an electron injection and transport layer exhibits excellent characteristics in terms of efficiency, driving voltage, and/or stability of the organic light emitting device.

Compared to commonly used electron injection and transport layers, the electron injection and transport layer of the present invention exhibited low voltage and high efficiency characteristics by appropriately maintaining the distance between N-containing ring groups and limiting the linker linking direction which links the distance therebetween to the ortho to suitably cut the conjugation, thereby adjusting the electron injection and mobility. Furthermore, the electron injection and transport layer of the present invention also exhibited long-service life characteristics by introducing a CN-group having strong electronegativity to prevent electrons from being excessively injected.

Although the preferred exemplary embodiments (an electron injection and transport layer) of the present invention have been described above, the present invention is not limited thereto, and various modifications can be made and carried out within the scope of the claims and the detailed description of the invention, and also fall within the scope of the invention.

1. A compound of Chemical Formula 1: 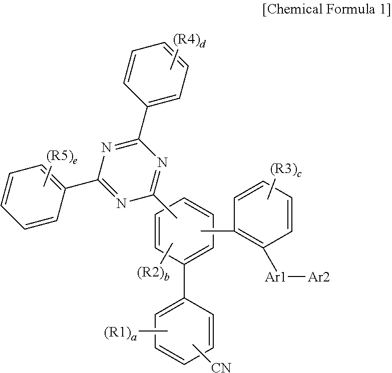
wherein, in Chemical Formula 1;
R1 to R5 are the same as or different from each other, and are each independently hydrogen, deuterium, a nitrile group, a halogen group, a substituted or unsubstituted alkyl group, a substituted or unsubstituted aryl group, or a substituted or unsubstituted heteroaryl group;
Ar1 is a substituted or unsubstituted heteroaryl group including two or more N atoms;
Ar2 is hydrogen, deuterium, a nitrile group, a halogen group, a substituted or unsubstituted alkyl group, a substituted or unsubstituted aryl group, a substituted or unsubstituted heteroaryl group, or a substituted or unsubstituted fused ring group;
a and c are an integer from 1 to 4;
b is an integer from 1 to 3;
d and e are an integer from 1 to 5;
when a is 2 or greater, the R1s are the same as or different from each other,
when b is 2 or greater, the R2s are the same as or different from each other,
when c is 2 or greater, the R3s are the same as or different from each other,
when d is 2 or greater, the R4s are the same as or different from each other, and
when e is 2 or greater, the R5s are the same as or different from each other.

2. The compound of claim 1, wherein Chemical Formula 1 is the following Chemical Formula 1-1 or 1-2: 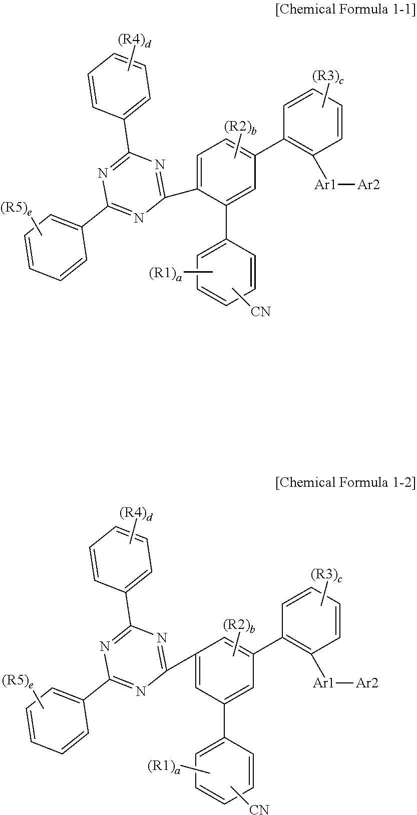
wherein in Chemical Formulae 1-1 and 1-2, R1 to R5, Ar1, Ar2, and a to e are the same as those defined in Chemical Formula 1.

3. The compound of claim 1, wherein Chemical Formula 1 is any one of the following Chemical Formulae 1-3 to 1-7: 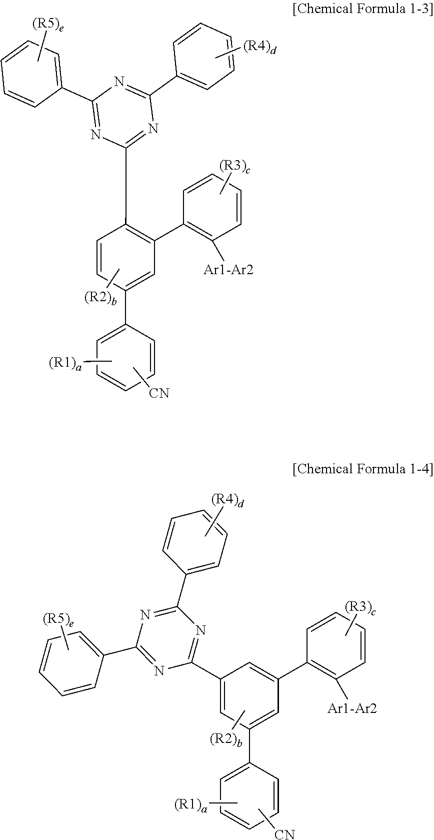 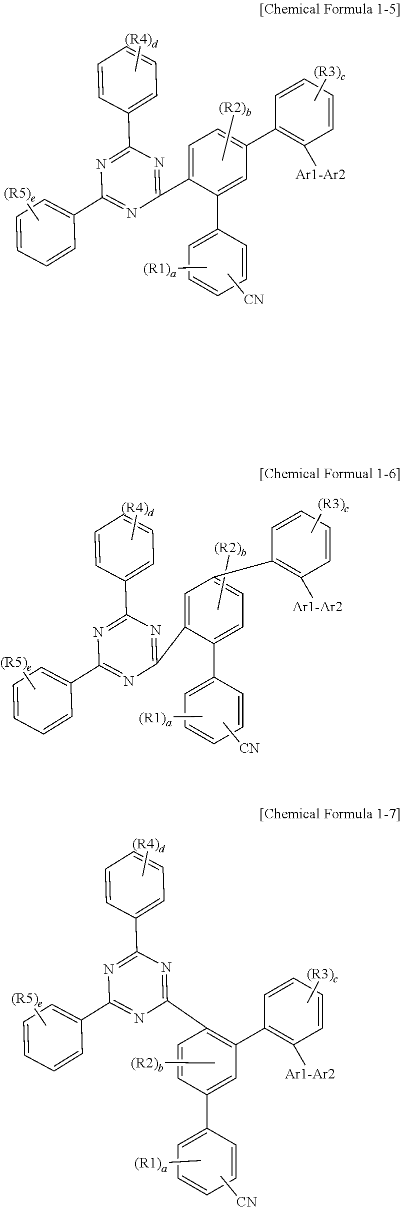
wherein in Chemical Formulae 1-3 to 1-7, R1 to R5, Ar1, Ar2, and a to e are the same as those defined in Chemical Formula 1.

4. The compound of claim 1, wherein Chemical Formula 1 is the following Chemical Formula 1-1-1 or 1-1-2: 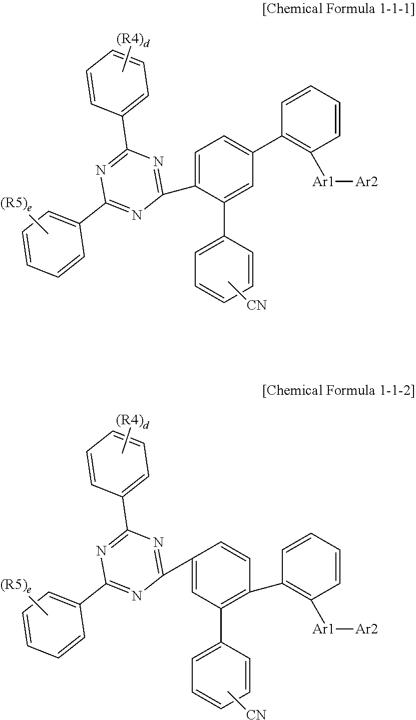
wherein in Chemical Formulae 1-1-1 and 1-1-2, R4, R5, Ar1, Ar2, d, and e are the same as those defined in Chemical Formula 1.

5. The compound of claim 1, wherein Chemical Formula 1 is any one of the following Chemical Formulae 1-1-3 to 1-1-7: 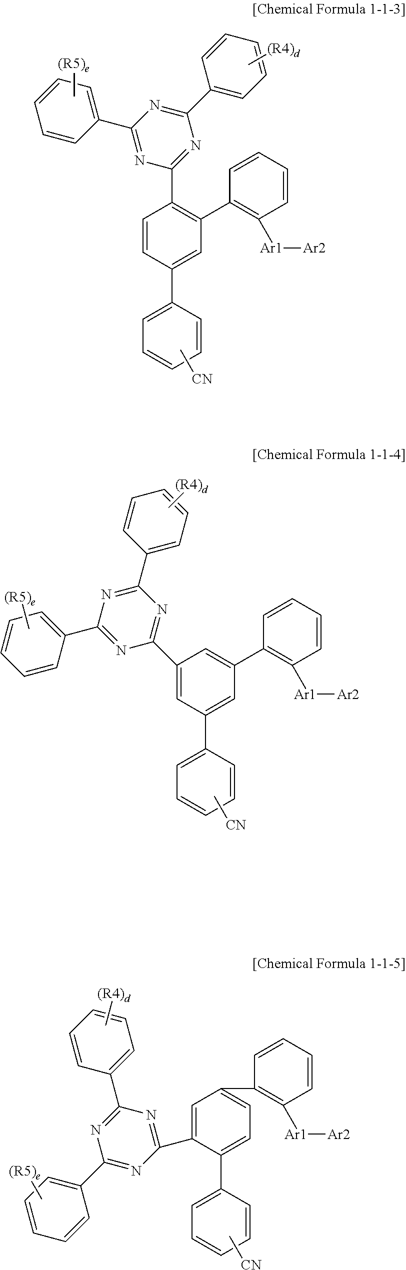 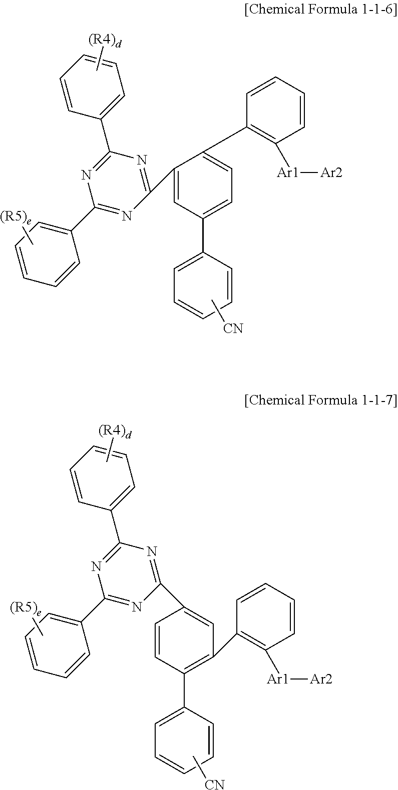
wherein in Chemical Formulae 1-1-3 to 1-1-7, R4, R5, Ar1, Ar2, d, and e are the same as those defined in Chemical Formula 1.

6. The compound of claim 1, wherein Ar1 is a triazine group.

7. The compound of claim 1, wherein Ar1 is a pyrimidine group.

8. The compound of claim 1, wherein Ar2 is an aryl group having 6 to 30 carbon atoms.

9. The compound of claim 1, wherein R1 to R5 are hydrogen or deuterium.

10. The compound of claim 1, wherein the compound of Chemical Formula 1 is any one of the following compounds:

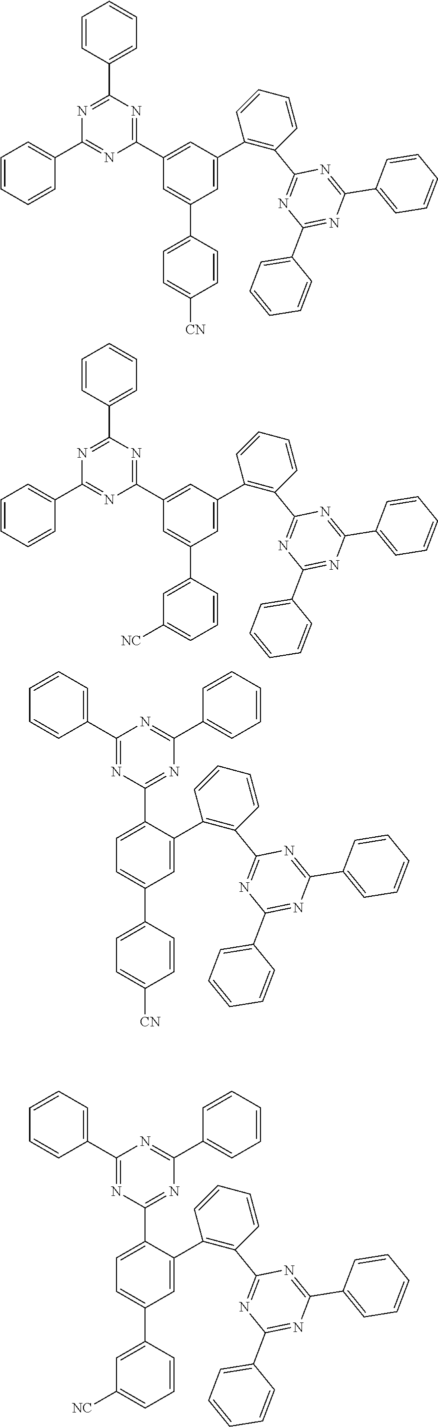

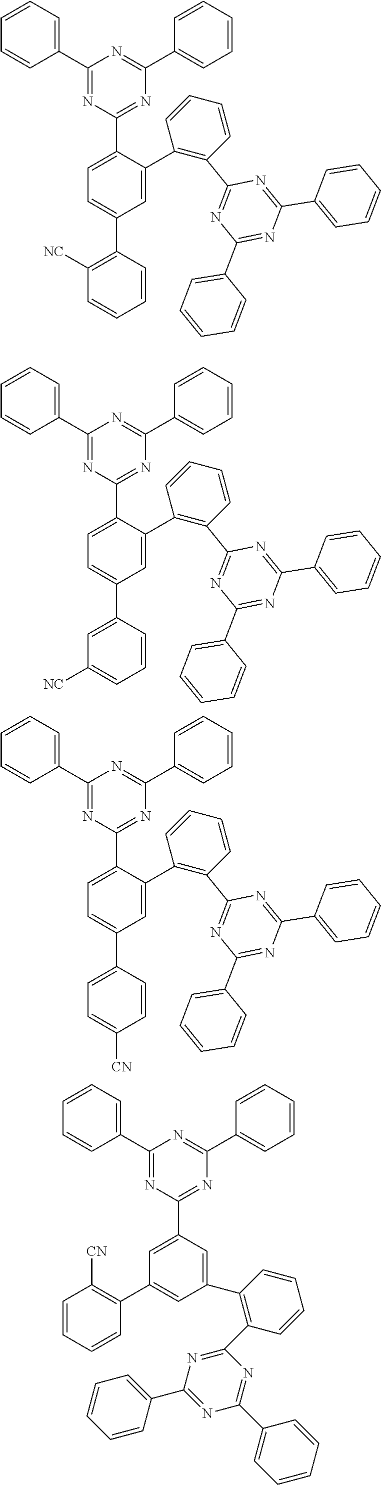

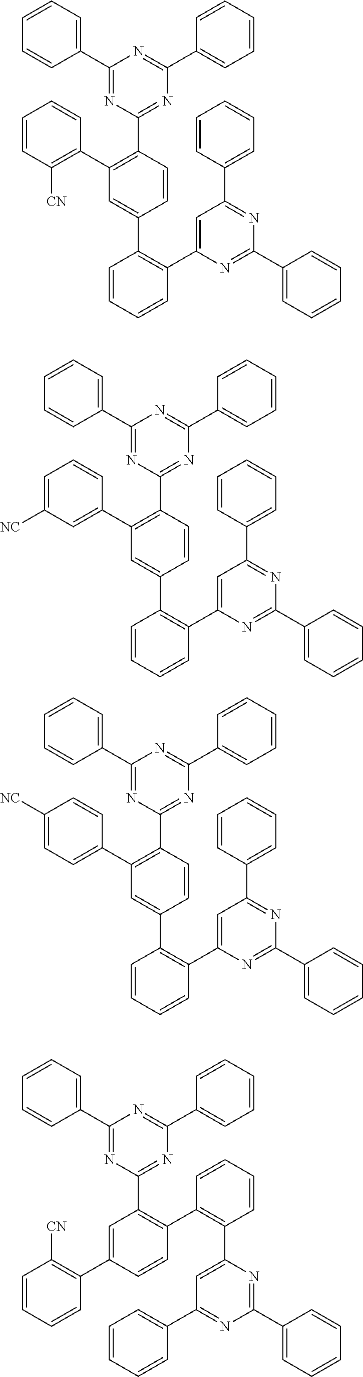 [SMILES: N#Cc1ccccc1-c1cc(-c2ccccc2-c2cc(-c3ccccc3)nc(-c3ccccc3)n2)ccc1-c1nc(-c2ccccc2)nc(-c2ccccc2)n1 ; N#Cc1cccc(-c2cc(-c3ccccc3-c3cc(-c4ccccc4)nc(-c4ccccc4)n3)ccc2-c2nc(-c3ccccc3)nc(-c3ccccc3)n2)c1 ; N#Cc1ccc(-c2cc(-c3ccccc3-c3cc(-c4ccccc4)nc(-c4ccccc4)n3)ccc2-c2nc(-c3ccccc3)nc(-c3ccccc3)n2)cc1 ; N#Cc1ccccc1-c1ccc(-c2ccccc2-c2cc(-c3ccccc3)nc(-c3ccccc3)n2)c(-c2nc(-c3ccccc3)nc(-c3ccccc3)n2)c1]

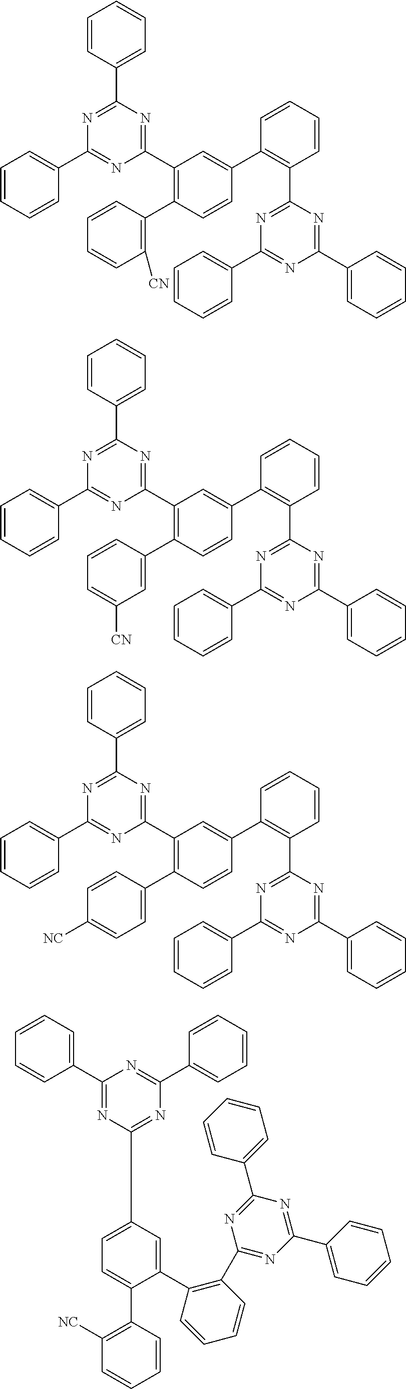

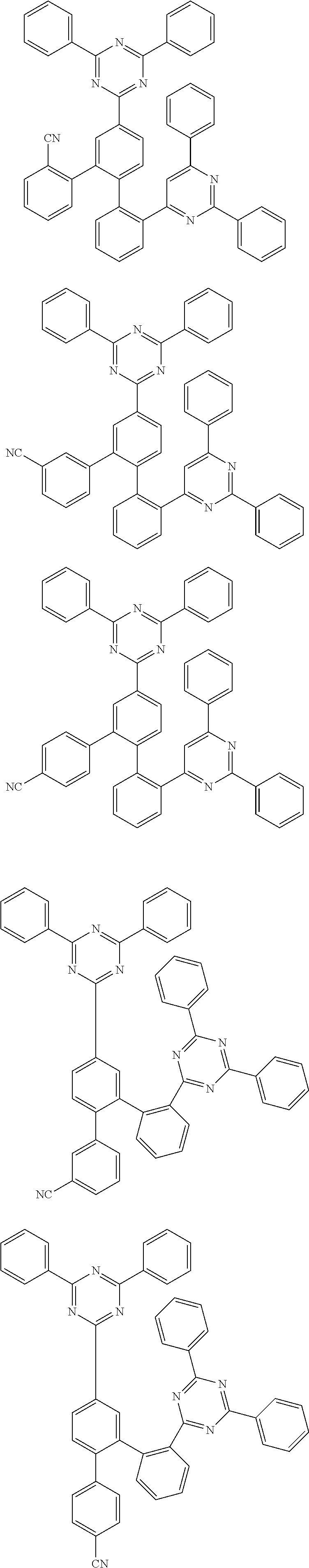 [SMILES: N#Cc1ccccc1-c1cc(-c2nc(-c3ccccc3)nc(-c3ccccc3)n2)ccc1-c1ccccc1-c1cc(-c2ccccc2)nc(-c2ccccc2)n1 ; N#Cc1cccc(-c2cc(-c3nc(-c4ccccc4)nc(-c4ccccc4)n3)ccc2-c2ccccc2-c2cc(-c3ccccc3)nc(-c3ccccc3)n2)c1 ; N#Cc1ccc(-c2cc(-c3nc(-c4ccccc4)nc(-c4ccccc4)n3)ccc2-c2ccccc2-c2cc(-c3ccccc3)nc(-c3ccccc3)n2)cc1 ; N#Cc1cccc(-c2ccc(-c3nc(-c4ccccc4)nc(-c4ccccc4)n3)cc2-c2ccccc2-c2nc(-c3ccccc3)nc(-c3ccccc3)n2)c1 ; N#Cc1ccc(-c2ccc(-c3nc(-c4ccccc4)nc(-c4ccccc4)n3)cc2-c2ccccc2-c2nc(-c3ccccc3)nc(-c3ccccc3)n2)cc1]

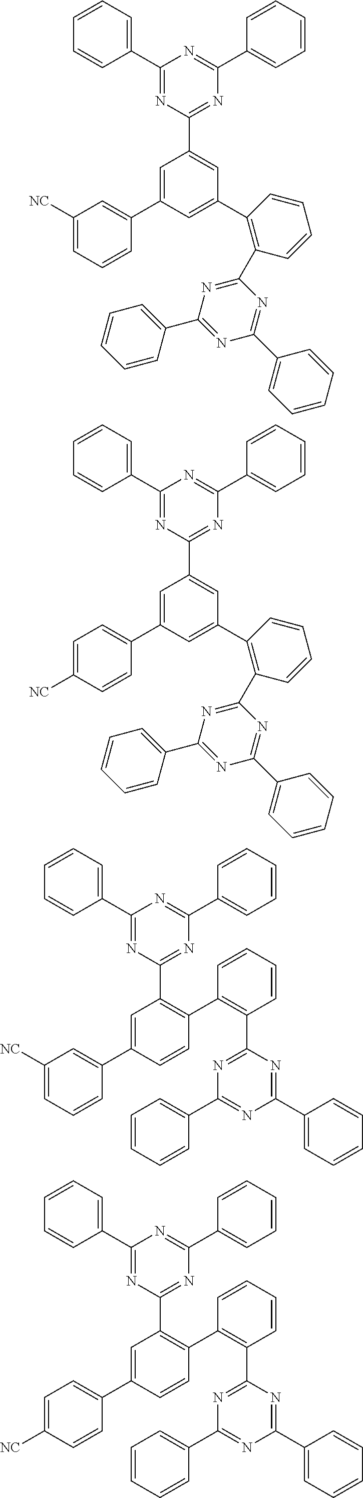

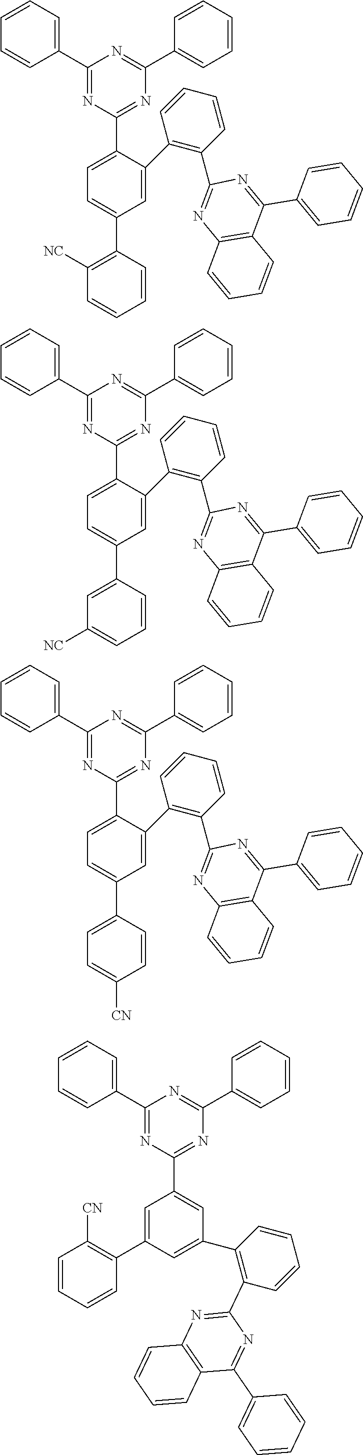

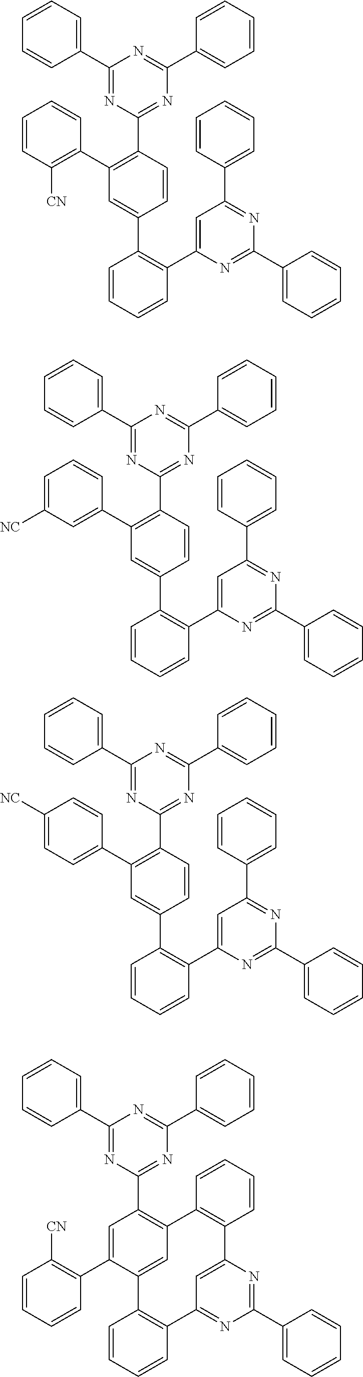 [SMILES: N#Cc1ccccc1-c1cc(-c2ccccc2-c2cc(-c3ccccc3)nc(-c3ccccc3)n2)ccc1-c1nc(-c2ccccc2)nc(-c2ccccc2)n1 ; N#Cc1cccc(-c2cc(-c3ccccc3-c3cc(-c4ccccc4)nc(-c4ccccc4)n3)ccc2-c2nc(-c3ccccc3)nc(-c3ccccc3)n2)c1 ; N#Cc1ccc(-c2cc(-c3ccccc3-c3cc(-c4ccccc4)nc(-c4ccccc4)n3)ccc2-c2nc(-c3ccccc3)nc(-c3ccccc3)n2)cc1 ; N#Cc1ccccc1-c1cc(-c2nc(-c3ccccc3)nc(-c3ccccc3)n2)c2cc1c1ccccc1c1cc(nc(-c3ccccc3)n1)c1ccccc21]

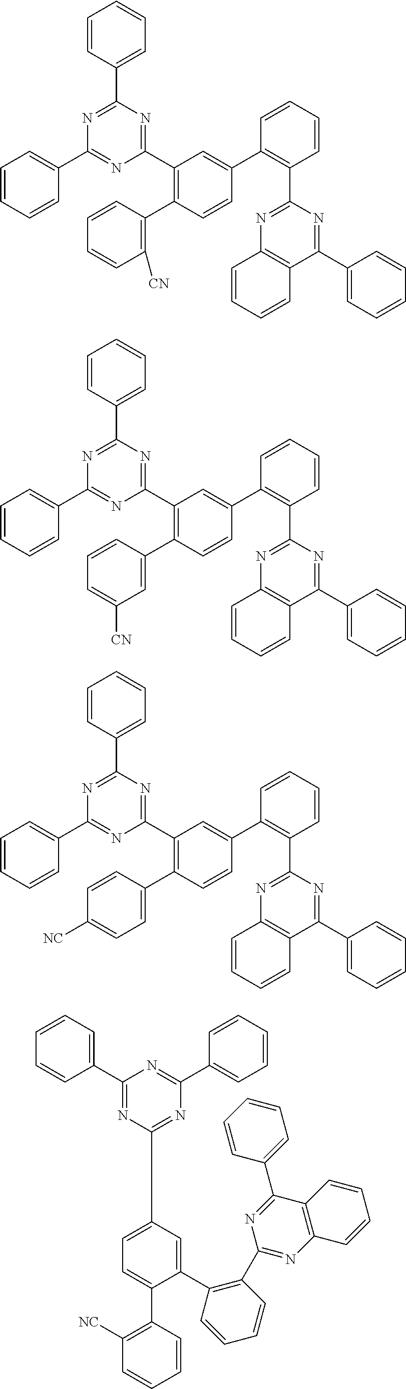

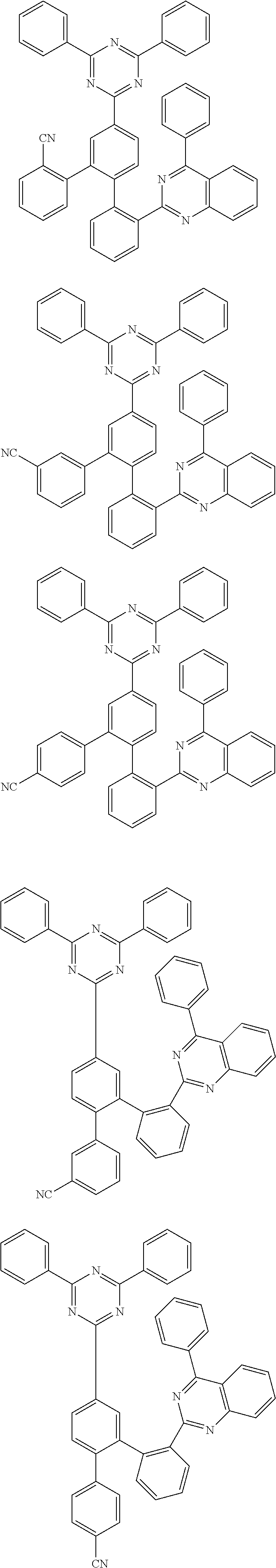 [SMILES: N#Cc1ccccc1-c1cc(-c2nc(-c3ccccc3)nc(-c3ccccc3)n2)ccc1-c1ccccc1-c1nc(-c2ccccc2)c2ccccc2n1 ; N#Cc1cccc(-c2cc(-c3nc(-c4ccccc4)nc(-c4ccccc4)n3)ccc2-c2ccccc2-c2nc(-c3ccccc3)c3ccccc3n2)c1 ; N#Cc1ccc(-c2cc(-c3nc(-c4ccccc4)nc(-c4ccccc4)n3)ccc2-c2ccccc2-c2nc(-c3ccccc3)c3ccccc3n2)cc1 ; N#Cc1cccc(-c2ccc(-c3nc(-c4ccccc4)nc(-c4ccccc4)n3)cc2-c2ccccc2-c2nc(-c3ccccc3)c3ccccc3n2)c1 ; N#Cc1ccc(-c2ccc(-c3nc(-c4ccccc4)nc(-c4ccccc4)n3)cc2-c2ccccc2-c2nc(-c3ccccc3)c3ccccc3n2)cc1]

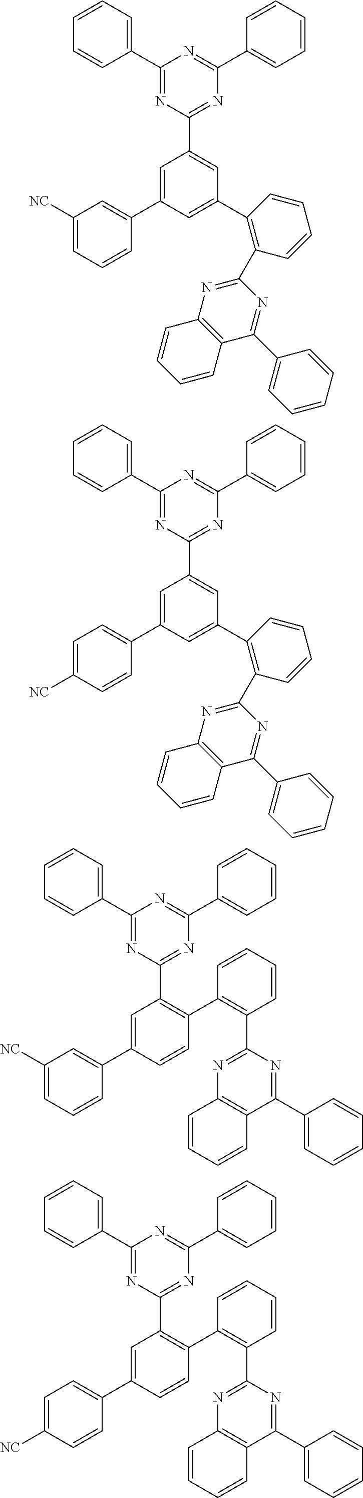

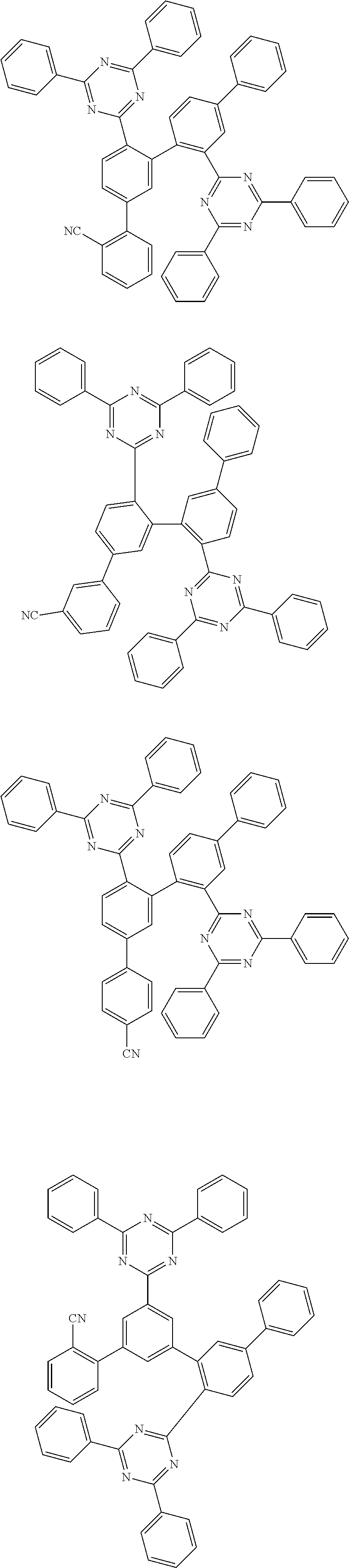 [SMILES: N#Cc1ccccc1-c1ccc(-c2nc(-c3ccccc3)nc(-c3ccccc3)n2)c(-c2ccc(-c3ccccc3)cc2-c2nc(-c3ccccc3)nc(-c3ccccc3)n2)c1 ; N#Cc1cccc(-c2ccc(-c3nc(-c4ccccc4)nc(-c4ccccc4)n3)c(-c3cc(-c4ccccc4)ccc3-c3nc(-c4ccccc4)nc(-c4ccccc4)n3)c2)c1 ; N#Cc1ccc(-c2ccc(-c3nc(-c4ccccc4)nc(-c4ccccc4)n3)c(-c3ccc(-c4ccccc4)cc3-c3nc(-c4ccccc4)nc(-c4ccccc4)n3)c2)cc1 ; N#Cc1ccccc1-c1cc(-c2nc(-c3ccccc3)nc(-c3ccccc3)n2)cc(-c2cc(-c3ccccc3)ccc2-c2nc(-c3ccccc3)nc(-c3ccccc3)n2)c1]

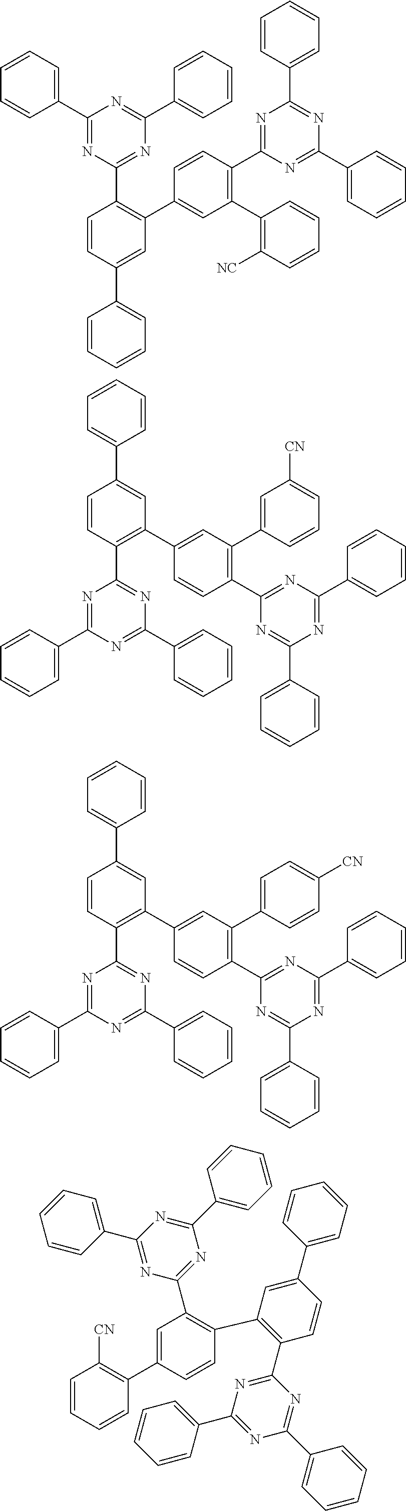

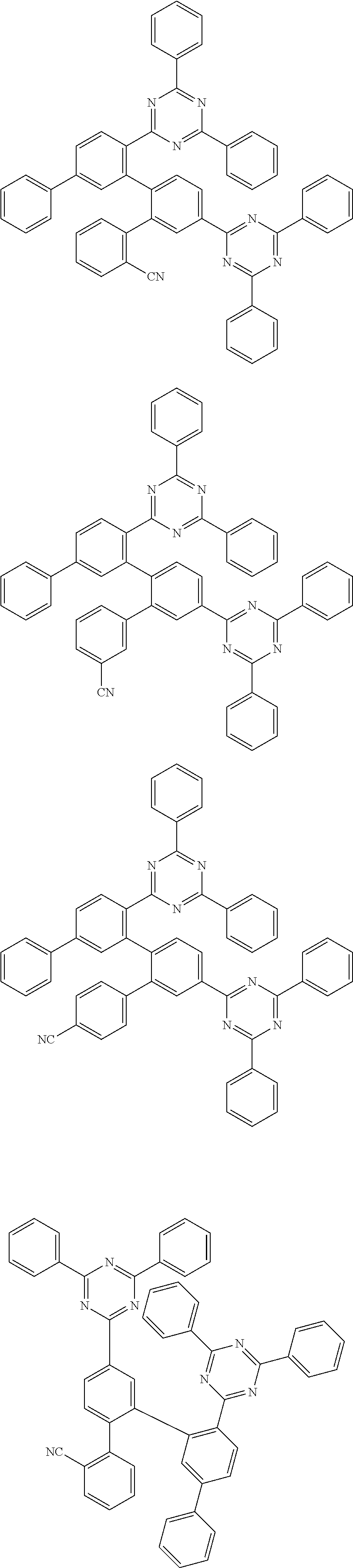 [SMILES: N#Cc1ccccc1-c1cc(-c2nc(-c3ccccc3)nc(-c3ccccc3)n2)ccc1-c1cc(-c2ccccc2)ccc1-c1nc(-c2ccccc2)nc(-c2ccccc2)n1 ; N#Cc1cccc(-c2cc(-c3nc(-c4ccccc4)nc(-c4ccccc4)n3)ccc2-c2cc(-c3ccccc3)ccc2-c2nc(-c3ccccc3)nc(-c3ccccc3)n2)c1 ; N#Cc1ccc(-c2cc(-c3nc(-c4ccccc4)nc(-c4ccccc4)n3)ccc2-c2cc(-c3ccccc3)ccc2-c2nc(-c3ccccc3)nc(-c3ccccc3)n2)cc1 ; N#Cc1ccccc1-c1ccc(-c2nc(-c3ccccc3)nc(-c3ccccc3)n2)cc1-c1cc(-c2ccccc2)ccc1-c1nc(-c2ccccc2)nc(-c2ccccc2)n1]

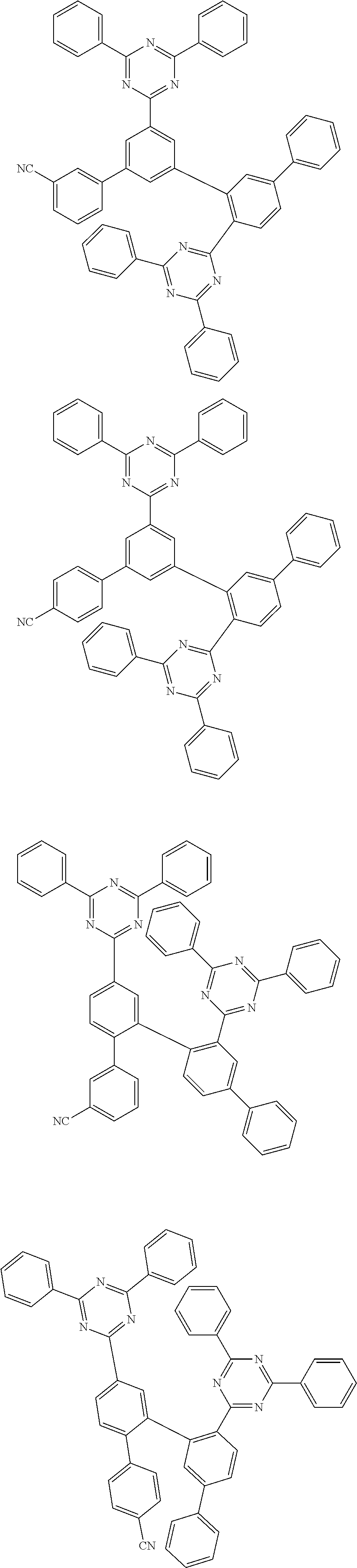

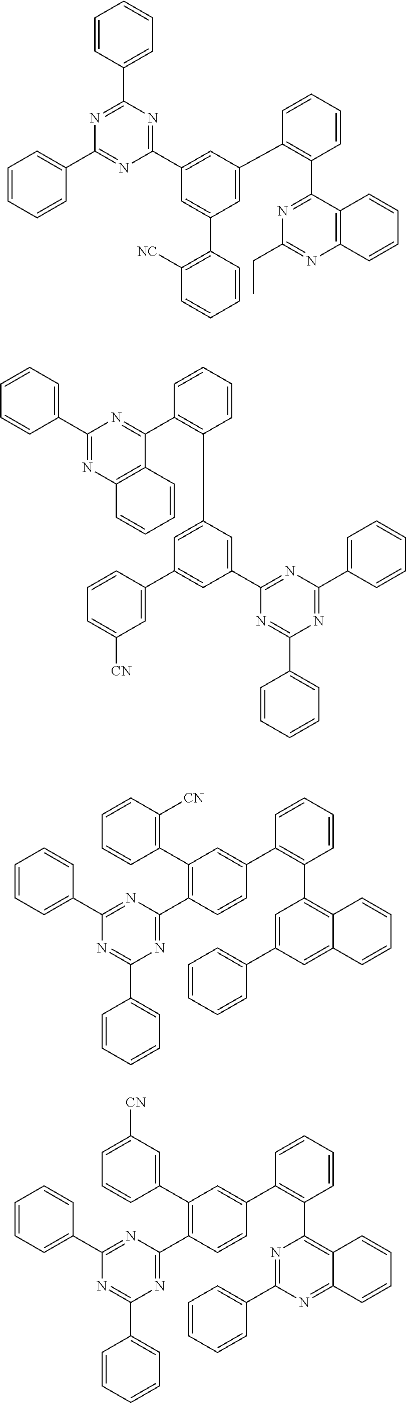

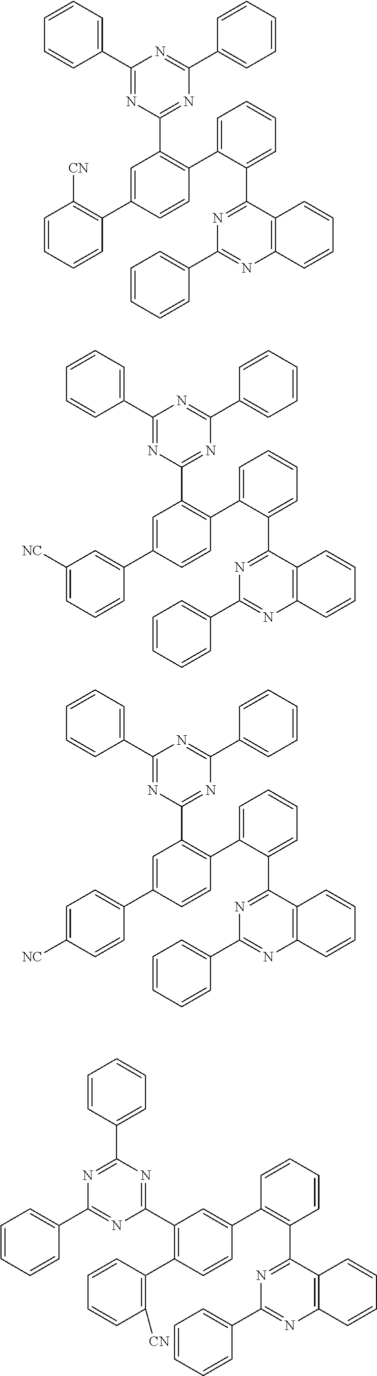 [SMILES: N#Cc1ccccc1-c1ccc(-c2ccccc2-c2nc(-c3ccccc3)nc3ccccc23)c(-c2nc(-c3ccccc3)nc(-c3ccccc3)n2)c1 ; N#Cc1cccc(-c2ccc(-c3ccccc3-c3nc(-c4ccccc4)nc4ccccc34)c(-c3nc(-c4ccccc4)nc(-c4ccccc4)n3)c2)c1 ; N#Cc1ccc(-c2ccc(-c3ccccc3-c3nc(-c4ccccc4)nc4ccccc34)c(-c3nc(-c4ccccc4)nc(-c4ccccc4)n3)c2)cc1 ; N#Cc1ccccc1-c1ccc(-c2ccccc2-c2nc(-c3ccccc3)nc3ccccc23)cc1-c1nc(-c2ccccc2)nc(-c2ccccc2)n1]

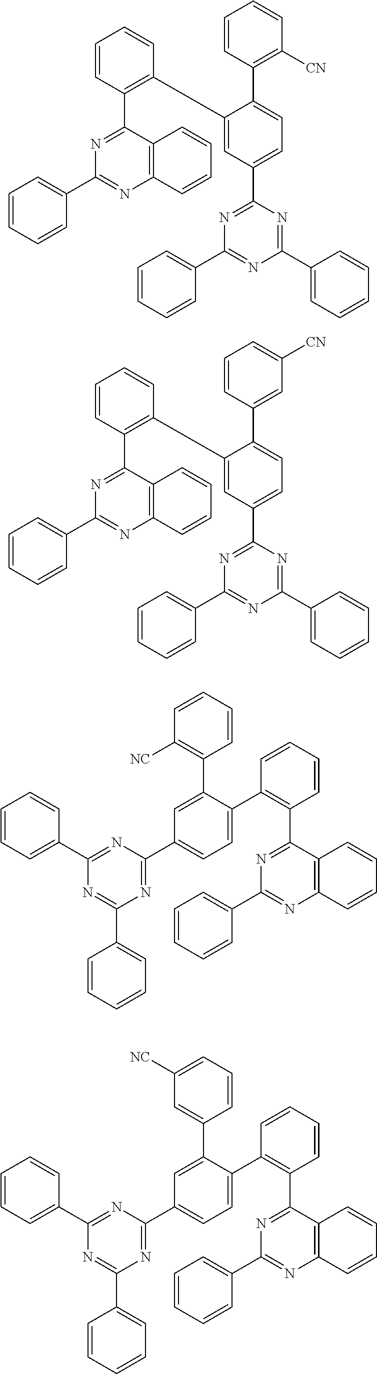

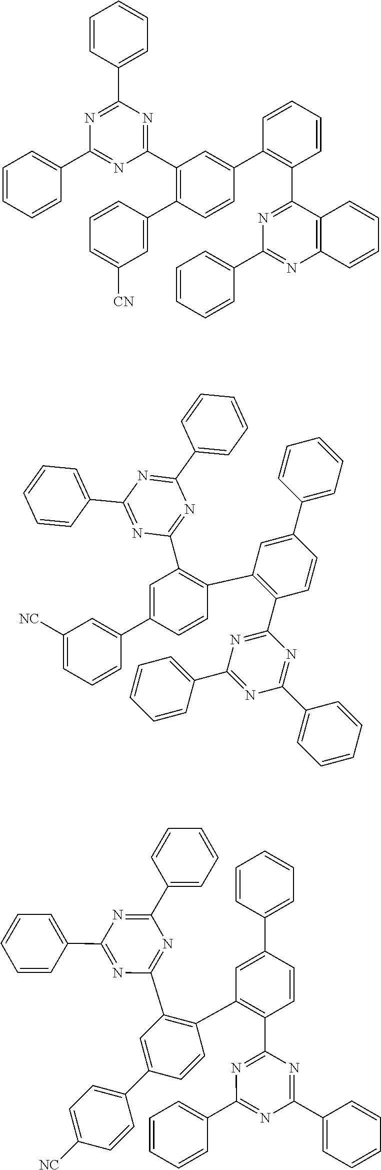 [SMILES: N#Cc1cccc(-c2ccc(-c3ccccc3-c3nc(-c4ccccc4)nc4ccccc34)cc2-c2nc(-c3ccccc3)nc(-c3ccccc3)n2)c1 ; N#Cc1cccc(-c2ccc(-c3cc(-c4ccccc4)ccc3-c3nc(-c4ccccc4)nc(-c4ccccc4)n3)c(-c3nc(-c4ccccc4)nc(-c4ccccc4)n3)c2)c1 ; N#Cc1ccc(-c2ccc(-c3cc(-c4ccccc4)ccc3-c3nc(-c4ccccc4)nc(-c4ccccc4)n3)c(-c3nc(-c4ccccc4)nc(-c4ccccc4)n3)c2)cc1]

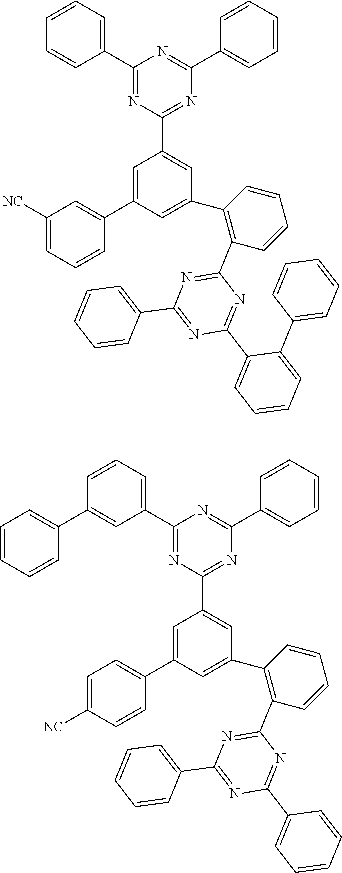 [SMILES: N#Cc1cccc(-c2cc(-c3nc(-c4ccccc4)nc(-c4ccccc4)n3)cc(-c3ccccc3-c3nc(-c4ccccc4)nc(-c4ccccc4-c4ccccc4)n3)c2)c1 ; N#Cc1ccc(-c2cc(-c3nc(-c4ccccc4)nc(-c4cccc(-c5ccccc5)c4)n3)cc(-c3ccccc3-c3nc(-c4ccccc4)nc(-c4ccccc4)n3)c2)cc1]

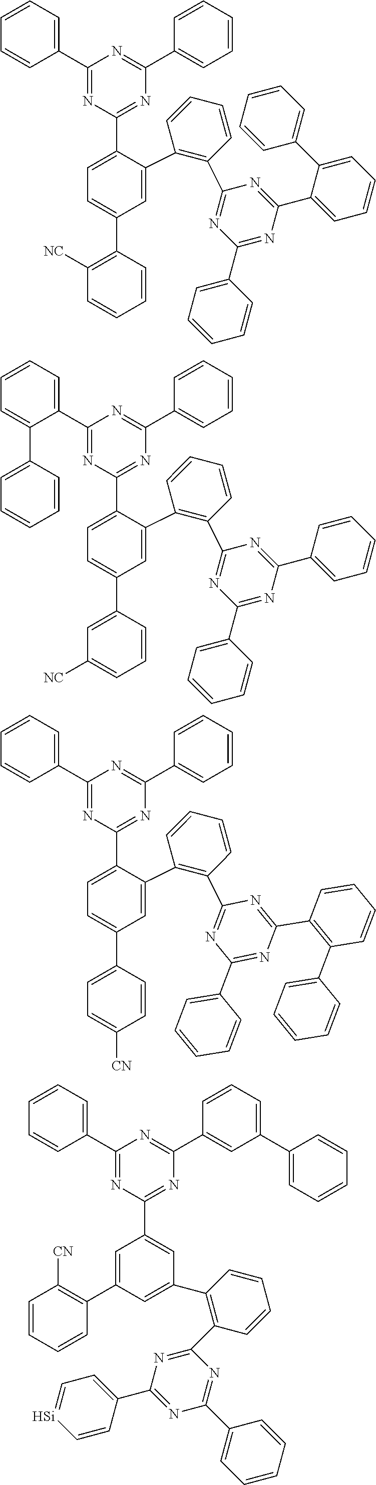

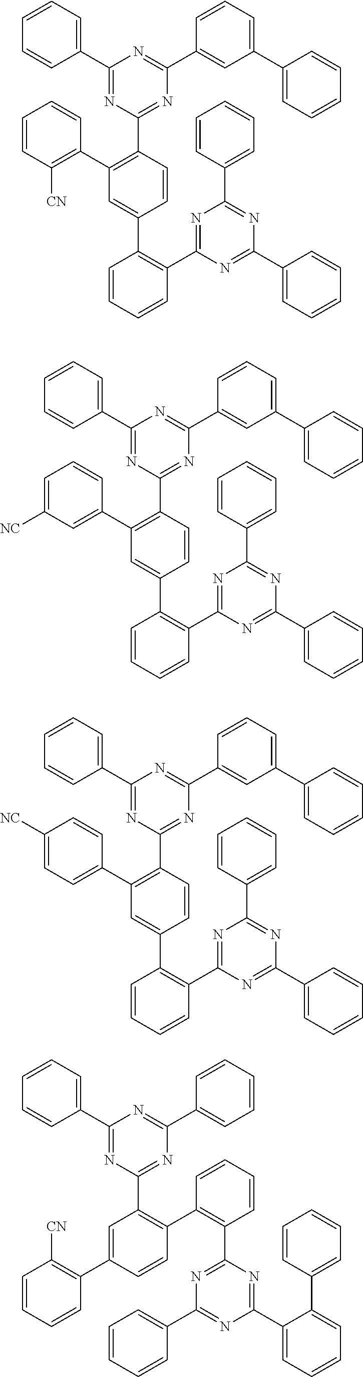 [SMILES: N#Cc1ccccc1-c1cc(-c2ccccc2-c2nc(-c3ccccc3)nc(-c3ccccc3)n2)ccc1-c1nc(-c2ccccc2)nc(-c2cccc(-c3ccccc3)c2)n1 ; N#Cc1cccc(-c2cc(-c3ccccc3-c3nc(-c4ccccc4)nc(-c4ccccc4)n3)ccc2-c2nc(-c3ccccc3)nc(-c3cccc(-c4ccccc4)c3)n2)c1 ; N#Cc1ccc(-c2cc(-c3ccccc3-c3nc(-c4ccccc4)nc(-c4ccccc4)n3)ccc2-c2nc(-c3ccccc3)nc(-c3cccc(-c4ccccc4)c3)n2)cc1 ; N#Cc1ccccc1-c1ccc(-c2ccccc2-c2nc(-c3ccccc3)nc(-c3ccccc3-c3ccccc3)n2)c(-c2nc(-c3ccccc3)nc(-c3ccccc3)n2)c1]

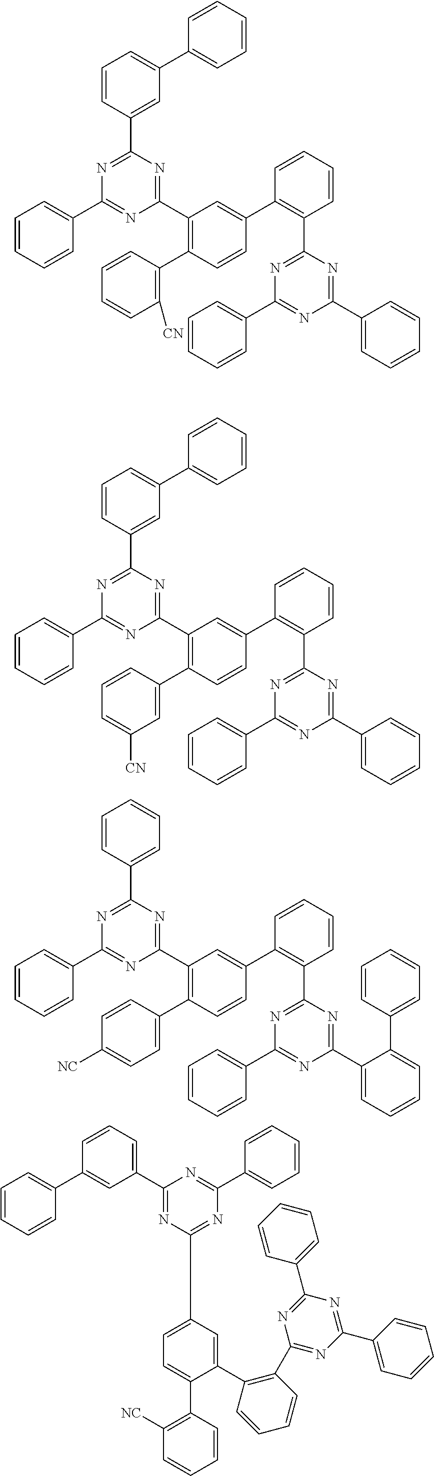

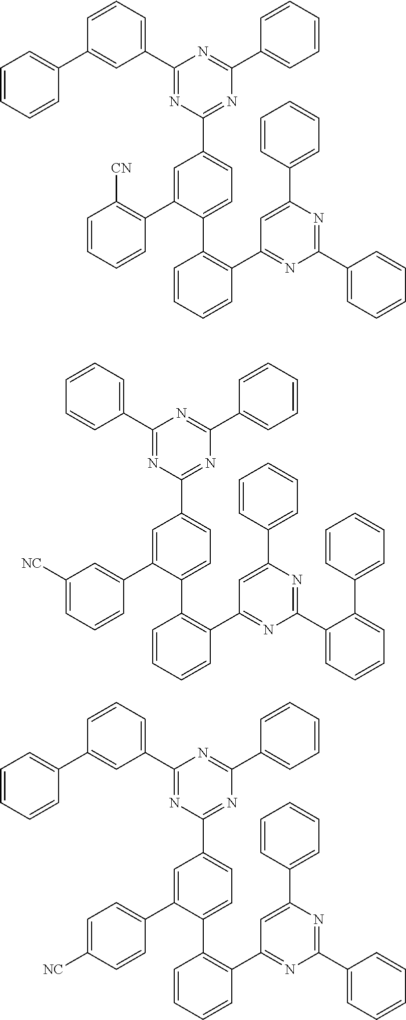 [SMILES: N#Cc1ccccc1-c1cc(-c2nc(-c3ccccc3)nc(-c3cccc(-c4ccccc4)c3)n2)ccc1-c1ccccc1-c1cc(-c2ccccc2)nc(-c2ccccc2)n1 ; N#Cc1cccc(-c2cc(-c3nc(-c4ccccc4)nc(-c4ccccc4)n3)ccc2-c2ccccc2-c2cc(-c3ccccc3)nc(-c3ccccc3-c3ccccc3)n2)c1 ; N#Cc1ccc(-c2cc(-c3nc(-c4ccccc4)nc(-c4cccc(-c5ccccc5)c4)n3)ccc2-c2ccccc2-c2cc(-c3ccccc3)nc(-c3ccccc3)n2)cc1]

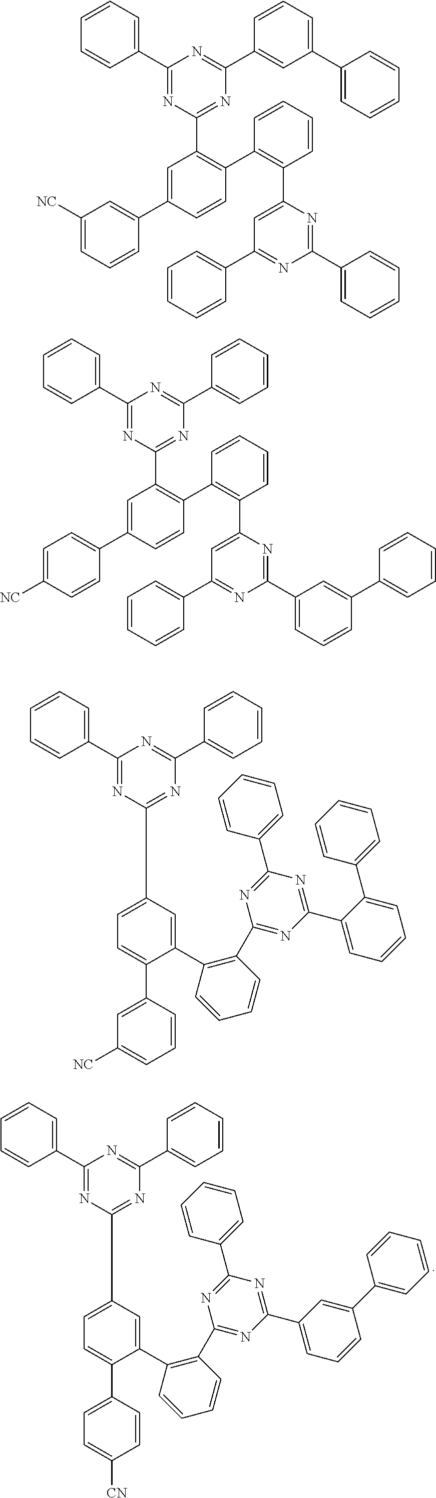

11. An organic light emitting device comprising:
a first electrode;
a second electrode provided to face the first electrode; and
an organic material layer having one or more layers provided between the first electrode and the second electrode,
wherein one or more layers of the organic material layer comprise the compound of Chemical Formula 1 according to claim 1.

12. The organic light emitting device of claim 11, wherein the organic material layer comprises one or more layers of an electron transport layer, an electron injection layer, and an electron injection and transport layer, and one or more layers of the one or more layers comprise the compound of Chemical Formula 1.
